(19) United States
(12) Patent Application Publication
(10) Pub. No.: US 2017/0334934 A1
(43) Pub. Date: Nov. 23, 2017

(54) Macrocyclic picolinamides as fungicides

(71) Applicant: DOW AGROSCIENCES LLC, Indianapolis, IN (US)

(72) Inventors: Kyle A. DEKORVER, Lafayette, IN (US); John F. DAEUBLE, SR., Carmel, IN (US); Johnathan E. DELORBE, Manvel, TX (US); Jeremy WILMONT, Indianapolis, IN (US); Chenglin YAO, Westfield, IN (US); Kevin G. MEYER, Indianapolis, IN (US)

(73) Assignee: Dow AgroSciences LLC, Indianapolis, IN (US)

(21) Appl. No.: 15/522,132

(22) PCT Filed: Oct. 22, 2015
(86) PCT No.: PCT/US15/56905

§ 371 Date: Apr. 26, 2017

Related U.S. Application Data
(60) Provisional application No. 62/069,452, filed on Oct. 28, 2014.

(51) Int. Cl. C07F 7/18; A01N 55/00; A01N 43/24; A01N 43/40; C07D 405/12; C07D 321/00

(52) U.S. Cl. CPC C07F 7/1868; C07D 405/12; C07D 321/00; A01N 43/24; A01N 43/40; A01N 55/00

(57) ABSTRACT

This disclosure relates to macrocyclic picolinamides of Formula I and their use as fungicides.

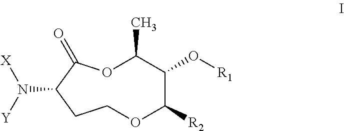

### CROSS REFERENCE TO RELATED APPLICATIONS

This application claims the benefit of U.S. Provisional Patent Application Ser. No. 62/069,452 filed Oct. 28, 2014, which is expressly incorporated by reference herein.

### BACKGROUND & SUMMARY

Fungicides are compounds, of natural or synthetic origin, which act to protect and/or cure plants against damage caused by agriculturally relevant fungi. Generally, no single fungicide is useful in all situations. Consequently, research is ongoing to produce fungicides that may have better performance, are easier to use, and cost less.

The present disclosure relates to macrocyclic picolinamides and their use as fungicides. The compounds of the present disclosure may offer protection against ascomycetes, basidiomycetes, deuteromycetes and oomycetes.

One embodiment of the present disclosure may include compounds of Formula I:

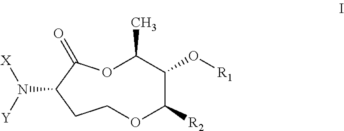

X is hydrogen or C(O)R₃;

Y is hydrogen, C(O)R₃, or Q;

Q is

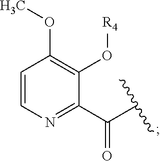

R₁ is hydrogen, alkyl, aryl, acyl, or silyl each optionally substituted with 0, 1 or multiple R₆;

R₂ is —(CH₂)ₙR₈ where n is an integer between 0 and 4, each optionally substituted with 0, 1 or multiple R₆;

R₃ is alkoxy or benzyloxy, each optionally substituted with 0, 1, or multiple R₆;

R₄ is hydrogen, —C(O)R₅, or —CH₂OC(O)R₅;

R₅ is alkyl, alkoxy, or aryl, each optionally substituted with 0, 1, or multiple R₆;

R₆ is hydrogen, alkyl, aryl, acyl, halo, alkenyl, alkoxy, heterocyclyl, or thioalkyl, each optionally substituted with 0, 1, or multiple R₇;

R₇ is hydrogen, alkyl, aryl, alkoxy, or halo.

R₈ is hydrogen, alkyl, alkenyl, aryl, heterocyclyl, or thioalkyl each substituted with 0, 1, or multiple R₆.

Another embodiment of the present disclosure may include a fungicidal composition for the control or prevention of fungal attack comprising the compounds described above and a phytologically acceptable carrier material.

Yet another embodiment of the present disclosure may include a method for the control or prevention of fungal attack on a plant, the method including the steps of applying a fungicidally effective amount of one or more of the compounds described above to at least one of the fungus, the plant, and an area adjacent to the plant.

It will be understood by those skilled in the art that the following terms may include generic “R”-groups within their definitions, e.g., “the term alkoxy refers to an —OR substituent”. It is also understood that within the definitions for the following terms, these “R” groups are included for illustration purposes and should not be construed as limiting or being limited by substitutions about Formula I.

The term “alkyl” refers to a branched, unbranched, or saturated cyclic carbon chain, including, but not limited to, methyl, ethyl, propyl, butyl, isopropyl, isobutyl, tertiary butyl, pentyl, hexyl, cyclopropyl, cyclobutyl, cyclopentyl, cyclohexyl, and the like.

The term “alkenyl” refers to a branched, unbranched or cyclic carbon chain containing one or more double bonds including, but not limited to, ethenyl, propenyl, butenyl, isopropenyl, isobutenyl, cyclobutenyl, cyclopentenyl, cyclohexenyl, and the like.

The term “alkynyl” refers to a branched or unbranched carbon chain containing one or more triple bonds including, but not limited to, propynyl, butynyl, and the like.

The terms “aryl” and “Ar” refer to any aromatic ring, mono- or bi-cyclic, containing 0 heteroatoms.

The term “heterocyclyl” refers to any aromatic or non-aromatic ring, mono- or bi-cyclic, containing one or more heteroatoms

The term “alkoxy” refers to an —OR substituent.

The term “acyloxy” refers to an —OC(O)R substituent.

The term “cyano” refers to a —C≡N substituent.

The term “hydroxyl” refers to an —OH substituent.

The term “amino” refers to a —N(R)₂ substituent.

The term “arylalkoxy” refers to —O(CH₂)ₙAr where n is an integer selected from the list 1, 2, 3, 4, 5, or 6.

The term “haloalkoxy” refers to an —OR—X substituent, wherein X is Cl, F, Br, or I, or any combination thereof.

The term “haloalkyl” refers to an alkyl, which is substituted with Cl, F, I, or Br or any combination thereof.

The term “halogen” or “halo” refers to one or more halogen atoms, defined as F, Cl, Br, and I.

The term “nitro” refers to a —NO₂ substituent.

The term thioalkyl refers to an —SR substituent.

Throughout the disclosure, reference to the compounds of Formula I is read as also including diastereomers, enantiomers, and mixtures thereof. In another embodiment, Formula (I) is read as also including salts or hydrates thereof. Exemplary salts include, but are not limited to: hydrochloride, hydrobromide, and hydroiodide.

It is also understood by those skilled in the art that additional substitution is allowable, unless otherwise noted, as long as the rules of chemical bonding and strain energy are satisfied and the product still exhibits fungicidal activity.

Another embodiment of the present disclosure is a use of a compound of Formula I, for protection of a plant against attack by a phytopathogenic organism or the treatment of a plant infested by a phytopathogenic organism, comprising the application of a compound of Formula I, or a composition comprising the compound to soil, a plant, a part of a plant, foliage, and/or roots.

Additionally, another embodiment of the present disclosure is a composition useful for protecting a plant against attack by a phytopathogenic organism and/or treatment of a plant infested by a phytopathogenic organism comprising a compound of Formula I and a phytologically acceptable carrier material.

### DETAILED DESCRIPTION

The compounds of the present disclosure may be applied by any of a variety of known techniques, either as the compounds or as formulations comprising the compounds. For example, the compounds may be applied to the roots or foliage of plants for the control of various fungi, without damaging the commercial value of the plants. The materials may be applied in the form of any of the generally used formulation types, for example, as solutions, dusts, wettable powders, flowable concentrate, or emulsifiable concentrates.

Preferably, the compounds of the present disclosure are applied in the form of a formulation, comprising one or more of the compounds of Formula I with a phytologically acceptable carrier. Concentrated formulations may be dispersed in water, or other liquids, for application, or formulations may be dust-like or granular, which may then be applied without further treatment. The formulations can be prepared according to procedures that are conventional in the agricultural chemical art.

The present disclosure contemplates all vehicles by which one or more of the compounds may be formulated for delivery and use as a fungicide. Typically, formulations are applied as aqueous suspensions or emulsions. Such suspensions or emulsions may be produced from water-soluble, water-suspendible, or emulsifiable formulations which are solids, usually known as wettable powders; or liquids, usually known as emulsifiable concentrates, aqueous suspensions, or suspension concentrates. As will be readily appreciated, any material to which these compounds may be added may be used, provided it yields the desired utility without significant interference with the activity of these compounds as antifungal agents.

Wettable powders, which may be compacted to form water-dispersible granules, comprise an intimate mixture of one or more of the compounds of Formula I, an inert carrier and surfactants. The concentration of the compound in the wettable powder may be from about 10 percent to about 90 percent by weight based on the total weight of the wettable powder, more preferably about 25 weight percent to about 75 weight percent. In the preparation of wettable powder formulations, the compounds may be compounded with any finely divided solid, such as prophyllite, talc, chalk, gypsum, Fuller's earth, bentonite, attapulgite, starch, casein, gluten, montmorillonite clays, diatomaceous earths, purified silicates or the like. In such operations, the finely divided carrier and surfactants are typically blended with the compound(s) and milled.

Emulsifiable concentrates of the compounds of Formula I may comprise a convenient concentration, such as from about 1 weight percent to about 50 weight percent of the compound, in a suitable liquid, based on the total weight of the concentrate. The compounds may be dissolved in an inert carrier, which is either a water-miscible solvent or a mixture of water-immiscible organic solvents, and emulsifiers. The concentrates may be diluted with water and oil to form spray mixtures in the form of oil-in-water emulsions. Useful organic solvents include aromatics, especially the high-boiling naphthalenic and olefinic portions of petroleum such as heavy aromatic naphtha. Other organic solvents may also be used, for example, terpenic solvents, including rosin derivatives, aliphatic ketones, such as cyclohexanone, and complex alcohols, such as 2-ethoxyethanol.

Emulsifiers which may be advantageously employed herein may be readily determined by those skilled in the art and include various nonionic, anionic, cationic and amphoteric emulsifiers, or a blend of two or more emulsifiers. Examples of nonionic emulsifiers useful in preparing the emulsifiable concentrates include the polyalkylene glycol ethers and condensation products of alkyl and aryl phenols, aliphatic alcohols, aliphatic amines or fatty acids with ethylene oxide, propylene oxides such as the ethoxylated alkyl phenols and carboxylic esters solubilized with the polyol or polyoxyalkylene. Cationic emulsifiers include quaternary ammonium compounds and fatty amine salts. Anionic emulsifiers include the oil-soluble salts (e.g., calcium) of alkylaryl sulphonic acids, oil-soluble salts or sulfated polyglycol ethers and appropriate salts of phosphated polyglycol ether.

Representative organic liquids which may be employed in preparing the emulsifiable concentrates of the compounds of the present disclosure are the aromatic liquids such as xylene, propyl benzene fractions; or mixed naphthalene fractions, mineral oils, substituted aromatic organic liquids such as dioctyl phthalate; kerosene; dialkyl amides of various fatty acids, particularly the dimethyl amides of fatty glycols and glycol derivatives such as the n-butyl ether, ethyl ether or methyl ether of diethylene glycol, the methyl ether of triethylene glycol, petroleum fractions or hydrocarbons such as mineral oil, aromatic solvents, paraffinic oils, and the like; vegetable oils such as soy bean oil, rape seed oil, olive oil, castor oil, sunflower seed oil, coconut oil, corn oil, cotton seed oil, linseed oil, palm oil, peanut oil, safflower oil, sesame oil, tung oil and the like; esters of the above vegetable oils; and the like. Mixtures of two or more organic liquids may also be employed in the preparation of the emulsifiable concentrate. Organic liquids include xylene, and propyl benzene fractions, with xylene being most preferred in some cases. Surface-active dispersing agents are typically employed in liquid formulations and in an amount of from 0.1 to 20 percent by weight based on the combined weight of the dispersing agent with one or more of the compounds. The formulations can also contain other compatible additives, for example, plant growth regulators and other biologically active compounds used in agriculture.

Aqueous suspensions comprise suspensions of one or more water-insoluble compounds of Formula I, dispersed in an aqueous vehicle at a concentration in the range from about 1 to about 50 weight percent, based on the total weight of the aqueous suspension. Suspensions are prepared by finely grinding one or more of the compounds, and vigorously mixing the ground material into a vehicle comprised of water and surfactants chosen from the same types discussed above. Other components, such as inorganic salts and synthetic or natural gums, may also be added to increase the density and viscosity of the aqueous vehicle.

The compounds of Formula I can also be applied as granular formulations, which are particularly useful for applications to the soil. Granular formulations generally contain from about 0.5 to about 10 weight percent, based on the total weight of the granular formulation of the compound(s), dispersed in an inert carrier which consists entirely or in large part of coarsely divided inert material such as attapulgite, bentonite, diatomite, clay or a similar inexpensive substance. Such formulations are usually prepared by dissolving the compounds in a suitable solvent and applying it to a granular carrier which has been preformed to the appropriate particle size, in the range of from about 0.5 to about 3 mm. A suitable solvent is a solvent in which the compound is substantially or completely soluble. Such formulations may also be prepared by making a dough or paste of the carrier and the compound and solvent, and crushing and drying to obtain the desired granular particle.

Dusts containing the compounds of Formula I may be prepared by intimately mixing one or more of the compounds in powdered form with a suitable dusty agricultural carrier, such as, for example, kaolin clay, ground volcanic rock, and the like. Dusts can suitably contain from about 1 to about 10 weight percent of the compounds, based on the total weight of the dust.

The formulations may additionally contain adjuvant surfactants to enhance deposition, wetting, and penetration of the compounds onto the target crop and organism. These adjuvant surfactants may optionally be employed as a component of the formulation or as a tank mix. The amount of adjuvant surfactant will typically vary from 0.01 to 1.0 percent by volume, based on a spray-volume of water, preferably 0.05 to 0.5 volume percent. Suitable adjuvant surfactants include, but are not limited to ethoxylated nonyl phenols, ethoxylated synthetic or natural alcohols, salts of the esters or sulphosuccinic acids, ethoxylated organosilicones, ethoxylated fatty amines, blends of surfactants with mineral or vegetable oils, crop oil concentrate (mineral oil (85%)+emulsifiers (15%)); nonylphenol ethoxylate; benzylcocoalkyldimethyl quaternary ammonium salt; blend of petroleum hydrocarbon, alkyl esters, organic acid, and anionic surfactant; C₉-C₁₁ alkylpolyglycoside; phosphated alcohol ethoxylate; natural primary alcohol (C₁₂₋C₁₆) ethoxylate; di-sec-butylphenol EO-PO block copolymer; polysiloxane-methyl cap; nonylphenol ethoxylate+urea ammonium nitrrate; emulsified methylated seed oil; tridecyl alcohol (synthetic) ethoxylate (8EO); tallow amine ethoxylate (15 EO); PEG(400) dioleate-99. The formulations may also include oil-in-water emulsions such as those disclosed in U.S. patent application Ser. No. 11/495,228, the disclosure of which is expressly incorporated by reference herein.

The formulations may optionally include combinations that contain other pesticidal compounds. Such additional pesticidal compounds may be fungicides, insecticides, herbicides, nematocides, miticides, arthropodicides, bactericides or combinations thereof that are compatible with the compounds of the present disclosure in the medium selected for application, and not antagonistic to the activity of the present compounds. Accordingly, in such embodiments, the other pesticidal compound is employed as a supplemental toxicant for the same or for a different pesticidal use. The compounds of Formula I and the pesticidal compound in the combination can generally be present in a weight ratio of from 1:100 to 100:1.

The compounds of the present disclosure may also be combined with other fungicides to form fungicidal mixtures and synergistic mixtures thereof. The fungicidal compounds of the present disclosure are often applied in conjunction with one or more other fungicides to control a wider variety of undesirable diseases. When used in conjunction with other fungicide(s), the presently claimed compounds may be formulated with the other fungicide(s), tank-mixed with the other fungicide(s) or applied sequentially with the other fungicide(s). Such other fungicides may include 2-(thiocyanatomethylthio)-benzothiazole, 2-phenylphenol, 8-hydroxyquinoline sulfate, ametoctradin, amisulbrom, antimycin, Ampelomyces quisqualis, azaconazole, azoxystrobin, Bacillus subtilis, Bacillus subtilis strain QST713, benalaxyl, benomyl, benthiavalicarb-isopropyl, benzovindiflupyr benzylaminobenzene-sulfonate (BABS) salt, bicarbonates, biphenyl, bismerthiazol, bitertanol, bixafen, blasticidin-S, borax, Bordeaux mixture, boscalid, bromuconazole, bupirimate, calcium polysulfide, captafol, captan, carbendazim, carboxin, carpropamid, carvone, chlazafenone, chloroneb, chlorothalonil, chlozolinate, Coniothyrium minitans, copper hydroxide, copper octanoate, copper oxychloride, copper sulfate, copper sulfate (tribasic), cuprous oxide, cyazofamid, cyflufenamid, cymoxanil, cyproconazole, cyprodinil, dazomet, debacarb, diammonium ethylenebis-(dithiocarbamate), dichlofluanid, dichlorophen, diclocymet, diclomezine, dichloran, diethofencarb, difenoconazole, difenzoquat ion, diflumetorim, dimethomorph, dimoxystrobin, diniconazole, diniconazole-M, dinobuton, dinocap, diphenylamine, dithianon, dodemorph, dodemorph acetate, dodine, dodine free base, edifenphos, enestrobin, enestroburin, epoxiconazole, ethaboxam, ethoxyquin, etridiazole, famoxadone, fenamidone, fenarimol, fenbuconazole, fenfuram, fenhexamid, fenoxanil, fenpiclonil, fenpropidin, fenpropimorph, fenpyrazamine, fentin, fentin acetate, fentin hydroxide, ferbam, ferimzone, fluazinam, fludioxonil, flumorph, fluopicolide, fluopyram, fluoroimide, fluoxastrobin, fluquinconazole, flusilazole, flusulfamide, flutianil, flutolanil, flutriafol, fluxapyroxad, folpet, formaldehyde, fosetyl, fosetyl-aluminium, fuberidazole, furalaxyl, furametpyr, guazatine, guazatine acetates, GY-81, hexachlorobenzene, hexaconazole, hymexazol, imazalil, imazalil sulfate, imibenconazole, iminoctadine, iminoctadine triacetate, iminoctadine tris(albesilate), iodocarb, ipconazole, ipfenpyrazolone, iprobenfos, iprodione, iprovalicarb, isoprothiolane, isopyrazam, isotianil, kasugamycin, kasugamycin hydrochloride hydrate, kresoxim-methyl, laminarin, mancopper, mancozeb, mandipropamid, maneb, mefenoxam, mepanipyrim, mepronil, meptyl-dinocap, mercuric chloride, mercuric oxide, mercurous chloride, metalaxyl, metalaxyl-M, metam, metam-ammonium, metam-potassium, metam-sodium, metconazole, methasulfocarb, methyl iodide, methyl isothiocyanate, metiram, metominostrobin, metrafenone, mildiomycin, myclobutanil, nabam, nitrothal-isopropyl, nuarimol, octhilinone, ofurace, oleic acid (fatty acids), orysastrobin, oxadixyl, oxine-copper, oxpoconazole fumarate, oxycarboxin, pefurazoate, penconazole, pencycuron, penflufen, pentachlorophenol, pentachlorophenyl laurate, penthiopyrad, phenylmercury acetate, phosphonic acid, phthalide, picoxystrobin, polyoxin B, polyoxins, polyoxorim, potassium bicarbonate, potassium hydroxyquinoline sulfate, probenazole, prochloraz, procymidone, propamocarb, propamocarb hydrochloride, propiconazole, propineb, proquinazid, prothioconazole, pyraclostrobin, pyrametostrobin, pyraoxystrobin, pyrazophos, pyribencarb, pyributicarb, pyrifenox, pyrimethanil, pyriofenone, pyroquilon, quinoclamine, quinoxyfen, quintozene, Reynoutria sachalinensis extract, sedaxane, silthiofam, simeconazole, sodium 2-phenylphenoxide, sodium bicarbonate, sodium pentachlorophenoxide, spiroxamine, sulfur, SYP-Z048, tar oils, tebuconazole, tebufloquin, tecnazene, tetraconazole, thiabendazole, thifluzamide, thiophanate-methyl, thiram, tiadinil, tolclofos-methyl, tolylfluanid, triadimefon, triadimenol, triazoxide, tricyclazole, tridemorph, trifloxystrobin, triflumizole, triforine, triticonazole, validamycin, valifenalate, valiphenal, vinclozolin, zineb, ziram, zoxamide, Candida oleophila, Fusarium oxysporum, Gliocladium spp., Phlebiopsis gigantea, Streptomyces griseoviridis, Trichoderma spp., (RS)—N-(3,5-dichlorophenyl)-2-(methoxymethyl)-succinimide, 1,2-dichloropropane, 1,3-dichloro-1,1,3,3-tetrafluoroacetone hydrate, 1-chloro-2,4-dinitronaphthalene, 1-chloro-2-nitropropane, 2-(2-heptadecyl-2-imidazolin-1-yl)ethanol, 2,3-dihydro-5-phenyl-1,4-dithi-ine 1,1,4,4-tetraoxide, 2-methoxyethylmercury acetate, 2-methoxyethylmercury chloride, 2-methoxyethylmercury silicate, 3-(4-chlorophenyl)-5-methylrhodanine, 4-(2-nitroprop-1-enyl)phenyl thiocyanateme, ampropylfos, anilazine, azithiram, barium polysulfide, Bayer 32394, benodanil, benquinox, bentaluron, benzamacril; benzamacril-isobutyl, benzamorf, binapacryl, bis(methylmercury) sulfate, bis(tributyltin) oxide, buthiobate, cadmium calcium copper zinc chromate sulfate, carbamorph, CECA, chlobenthiazone, chloraniformethan, chlorfenazole, chlorquinox, climbazole, copper bis(3-phenylsalicylate), copper zinc chromate, cufraneb, cupric hydrazinium sulfate, cuprobam, cyclafuramid, cypendazole, cyprofuram, decafentin, dichlone, dichlozoline, diclobutrazol, dimethirimol, dinocton, dinosulfon, dinoterbon, dipyrithione, ditalimfos, dodicin, drazoxolon, EBP, ESBP, etaconazole, etem, ethirim, fenaminosulf, fenapanil, fenitropan, fluotrimazole, furcarbanil, furconazole, furconazole-cis, furmecyclox, furophanate, glyodine, griseofulvin, halacrinate, Hercules 3944, hexylthiofos, ICIA0858, isopamphos, isovaledione, mebenil, mecarbinzid, metazoxolon, methfuroxam, methylmercury dicyandiamide, metsulfovax, milneb, mucochloric anhydride, myclozolin, N-3,5-dichlorophenyl-succinimide, N-3-nitrophenylitaconimide, natamycin, N-ethylmercurio-4-toluenesulfonanilide, nickel bis(dimethyldithiocarbamate), OCH, phenylmercury dimethyldithiocarbamate, phenylmercury nitrate, phosdiphen, prothiocarb; prothiocarb hydrochloride, pyracarbolid, pyridinitril, pyroxychlor, pyroxyfur, quinacetol; quinacetol sulfate, quinazamid, quinconazole, rabenzazole, salicylanilide, SSF-109, sultropen, tecoram, thiadifluor, thicyofen, thiochlorfenphim, thiophanate, thioquinox, tioxymid, triamiphos, triarimol, triazbutil, trichlamide, urbacid, zarilamid, and any combinations thereof.

Additionally, the compounds described herein may be combined with other pesticides, including insecticides, nematocides, miticides, arthropodicides, bactericides or combinations thereof that are compatible with the compounds of the present disclosure in the medium selected for application, and not antagonistic to the activity of the present compounds to form pesticidal mixtures and synergistic mixtures thereof. The fungicidal compounds of the present disclosure may be applied in conjunction with one or more other pesticides to control a wider variety of undesirable pests. When used in conjunction with other pesticides, the presently claimed compounds may be formulated with the other pesticide(s), tank-mixed with the other pesticide(s) or applied sequentially with the other pesticide(s). Typical insecticides include, but are not limited to: 1,2-dichloropropane, abamectin, acephate, acetamiprid, acethion, acetoprole, acrinathrin, acrylonitrile, alanycarb, aldicarb, aldoxycarb, aldrin, allethrin, allosamidin, allyxycarb, alpha-cypermethrin, alpha-ecdysone, alpha-endosulfan, amidithion, aminocarb, amiton, amiton oxalate, amitraz, anabasine, athidathion, azadirachtin, azamethiphos, azinphos-ethyl, azinphos-methyl, azothoate, barium hexafluorosilicate, barthrin, bendiocarb, benfuracarb, bensultap, beta-cyfluthrin, beta-cypermethrin, bifenthrin, bioallethrin, bioethanomethrin, biopermethrin, bistrifluron, borax, boric acid, bromfenvinfos, bromocyclen, bromo-DDT, bromophos, bromophos-ethyl, bufencarb, buprofezin, butacarb, butathiofos, butocarboxim, butonate, butoxycarboxim, cadusafos, calcium arsenate, calcium polysulfide, camphechlor, carbanolate, carbaryl, carbofuran, carbon disulfide, carbon tetrachloride, carbophenothion, carbosulfan, cartap, cartap hydrochloride, chlorantraniliprole, chlorbicyclen, chlordane, chlordecone, chlordimeform, chlordimeform hydrochloride, chlorethoxyfos, chlorfenapyr, chlorfenvinphos, chlorfluazuron, chlormephos, chloroform, chloropicrin, chlorphoxim, chlorprazophos, chlorpyrifos, chlorpyrifos-methyl, chlorthiophos, chromafenozide, cinerin I, cinerin II, cinerins, cismethrin, cloethocarb, closantel, clothianidin, copper acetoarsenite, copper arsenate, copper naphthenate, copper oleate, coumaphos, coumithoate, crotamiton, crotoxyphos, crufomate, cryolite, cyanofenphos, cyanophos, cyanthoate, cyantraniliprole, cyclethrin, cycloprothrin, cyfluthrin, cyhalothrin, cypermethrin, cyphenothrin, cyromazine, cythioate, DDT, decarbofuran, deltamethrin, demephion, demephion-O, demephion-S, demeton, demeton-methyl, demeton-O, demeton-O-methyl, demeton-S, demeton-S-methyl, demeton-S-methylsulphon, diafenthiuron, dialifos, diatomaceous earth, diazinon, dicapthon, dichlofenthion, dichlorvos, dicresyl, dicrotophos, dicyclanil, dieldrin, diflubenzuron, dilor, dimefluthrin, dimefox, dimetan, dimethoate, dimethrin, dimethylvinphos, dimetilan, dinex, dinex-diclexine, dinoprop, dinosam, dinotefuran, diofenolan, dioxabenzofos, dioxacarb, dioxathion, disulfoton, dithicrofos, d-limonene, DNOC, DNOC-ammonium, DNOC-potassium, DNOC-sodium, doramectin, ecdysterone, emamectin, emamectin benzoate, EMPC, empenthrin, endosulfan, endothion, endrin, EPN, epofenonane, eprinomectin, esdepalldthrine, esfenvalerate, etaphos, ethiofencarb, ethion, ethiprole, ethoate-methyl, ethoprophos, ethyl formate, ethyl-DDD, ethylene dibromide, ethylene dichloride, ethylene oxide, etofenprox, etrimfos, EXD, famphur, fenamiphos, fenazaflor, fenchlorphos, fenethacarb, fenfluthrin, fenitrothion, fenobucarb, fenoxacrim, fenoxycarb, fenpirithrin, fenpropathrin, fensulfothion, fenthion, fenthion-ethyl, fenvalerate, fipronil, flonicamid, flubendiamide, flucofuron, flucycloxuron, flucythrinate, flufenerim, flufenoxuron, flufenprox, fluvalinate, fonofos, formetanate, formetanate hydrochloride, formothion, formparanate, formparanate hydrochloride, fosmethilan, fospirate, fosthietan, furathiocarb, furethrin, gamma-cyhalothrin, gamma-HCH, halfenprox, halofenozide, HCH, HEOD, heptachlor, heptenophos, heterophos, hexaflumuron, HHDN, hydramethylnon, hydrogen cyanide, hydroprene, hyquincarb, imidacloprid, imiprothrin, indoxacarb, iodomethane, IPSP, isazofos, isobenzan, isocarbophos, isodrin, isofenphos, isofenphos-methyl, isoprocarb, isoprothiolane, isothioate, isoxathion, ivermectin, jasmolin I, jasmolin II, jodfenphos, juvenile hormone I, juvenile hormone II, juvenile hormone III, kelevan, kinoprene, lambda-cyhalothrin, lead arsenate, lepimectin, leptophos, lindane, lirimfos, lufenuron, lythidathion, malathion, malonoben, mazidox, mecarbam, mecarphon, menazon, mephosfolan, mercurous chloride, mesulfenfos, metaflumizone, methacrifos, methamidophos, methidathion, methiocarb, methocrotophos, methomyl, methoprene, methoxychlor, methoxyfenozide, methyl bromide, methyl isothiocyanate, methylchloroform, methylene chloride, metofluthrin, metolcarb, metoxadiazone, mevinphos, mexacarbate, milbemectin, milbemycin oxime, mipafox, mirex, molosultap, monocrotophos, monomehypo, monosultap, morphothion, moxidectin, naftalofos, naled, naphthalene, nicotine, nifluridide, nitenpyram, nithiazine, nitrilacarb, novaluron, noviflumuron, omethoate, oxamyl, oxydemeton-methyl, oxydeprofos, oxydisulfoton, para-dichlorobenzene, parathion, parathion-methyl, penfluron, pentachlorophenol, permethrin, phenkapton, phenothrin, phenthoate, phorate, phosalone, phosfolan, phosmet, phosnichlor, phosphamidon, phosphine, phoxim, phoxim-methyl, pirimetaphos, pirimicarb, pirimiphos-ethyl, pirimiphos-methyl, potassium arsenite, potassium thiocyanate, pp′-DDT, prallethrin, precocene I, precocene II, precocene III, primidophos, profenofos, profluralin, promacyl, promecarb, propaphos, propetamphos, propoxur, prothidathion, prothiofos, prothoate, protrifenbute, pyraclofos, pyrafluprole, pyrazophos, pyresmethrin, pyrethrin I, pyrethrin II, pyrethrins, pyridaben, pyridalyl, pyridaphenthion, pyrifluquinazon, pyrimidifen, pyrimitate, pyriprole, pyriproxyfen, quassia, quinalphos, quinalphos-methyl, quinothion, rafoxanide, resmethrin, rotenone, ryania, sabadilla, schradan, selamectin, silafluofen, silica gel, sodium arsenite, sodium fluoride, sodium hexafluorosilicate, sodium thiocyanate, sophamide, spinetoram, spinosad, spiromesifen, spirotetramat, sulcofuron, sulcofuron-sodium, sulfluramid, sulfotep, sulfoxaflor, sulfuryl fluoride, sulprofos, tau-fluvalinate, tazimcarb, TDE, tebufenozide, tebufenpyrad, tebupirimfos, teflubenzuron, tefluthrin, temephos, TEPP, terallethrin, terbufos, tetrachloroethane, tetrachlorvinphos, tetramethrin, tetramethylfluthrin, theta-cypermethrin, thiacloprid, thiamethoxam, thicrofos, thiocarboxime, thiocyclam, thiocyclam oxalate, thiodicarb, thiofanox, thiometon, thiosultap, thiosultap-disodium, thiosultap-monosodium, thuringiensin, tolfenpyrad, tralomethrin, transfluthrin, transpermethrin, triarathene, triazamate, triazophos, trichlorfon, trichlormetaphos-3, trichloronat, trifenofos, triflumuron, trimethacarb, triprene, vamidothion, vaniliprole, XMC, xylylcarb, zeta-cypermethrin, zolaprofos, and any combinations thereof.

Additionally, the compounds described herein may be combined with herbicides that are compatible with the compounds of the present disclosure in the medium selected for application, and not antagonistic to the activity of the present compounds to form pesticidal mixtures and synergistic mixtures thereof. The fungicidal compounds of the present disclosure may be applied in conjunction with one or more herbicides to control a wide variety of undesirable plants. When used in conjunction with herbicides, the presently claimed compounds may be formulated with the herbicide(s), tank-mixed with the herbicide(s) or applied sequentially with the herbicide(s). Typical herbicides include, but are not limited to: 4-CPA; 4-CPB; 4-CPP; 2,4-D; 3,4-DA; 2,4-DB; 3,4-DB; 2,4-DEB; 2,4-DEP; 3,4-DP; 2,3,6-TBA; 2,4,5-T; 2,4,5-TB; acetochlor, acifluorfen, aclonifen, acrolein, alachlor, allidochlor, alloxydim, allyl alcohol, alorac, ametridione, ametryn, amibuzin, amicarbazone, amidosulfuron, aminocyclopyrachlor, aminopyralid, amiprofos-methyl, amitrole, ammonium sulfamate, anilofos, anisuron, asulam, atraton, atrazine, azafenidin, azimsulfuron, aziprotryne, barban, BCPC, beflubutamid, benazolin, bencarbazone, benfluralin, benfuresate, bensulfuron, bensulide, bentazone, benzadox, benzfendizone, benzipram, benzobicyclon, benzofenap, benzofluor, benzoylprop, benzthiazuron, bicyclopyrone, bifenox, bilanafos, bispyribac, borax, bromacil, bromobonil, bromobutide, bromofenoxim, bromoxynil, brompyrazon, butachlor, butafenacil, butamifos, butenachlor, buthidazole, buthiuron, butralin, butroxydim, buturon, butylate, cacodylic acid, cafenstrole, calcium chlorate, calcium cyanamide, cambendichlor, carbasulam, carbetamide, carboxazole chlorprocarb, carfentrazone, CDEA, CEPC, chlomethoxyfen, chloramben, chloranocryl, chlorazifop, chlorazine, chlorbromuron, chlorbufam, chloreturon, chlorfenac, chlorfenprop, chlorflurazole, chlorflurenol, chloridazon, chlorimuron, chlornitrofen, chloropon, chlorotoluron, chloroxuron, chloroxynil, chlorpropham, chlorsulfuron, chlorthal, chlorthiamid, cinidon-ethyl, cinmethylin, cinosulfuron, cisanilide, clethodim, cliodinate, clodinafop, clofop, clomazone, clomeprop, cloprop, cloproxydim, clopyralid, cloransulam, CMA, copper sulfate, CPMF, CPPC, credazine, cresol, cumyluron, cyanatryn, cyanazine, cycloate, cyclosulfamuron, cycloxydim, cycluron, cyhalofop, cyperquat, cyprazine, cyprazole, cypromid, daimuron, dalapon, dazomet, delachlor, desmedipham, desmetryn, di-allate, dicamba, dichlobenil, dichloralurea, dichlormate, dichlorprop, dichlorprop-P, diclofop, diclosulam, diethamquat, diethatyl, difenopenten, difenoxuron, difenzoquat, diflufenican, diflufenzopyr, dimefuron, dimepiperate, dimethachlor, dimethametryn, dimethenamid, dimethenamid-P, dimexano, dimidazon, dinitramine, dinofenate, dinoprop, dinosam, dinoseb, dinoterb, diphenamid, dipropetryn, diquat, disul, dithiopyr, diuron, DMPA, DNOC, DSMA, EBEP, eglinazine, endothal, epronaz, EPTC, erbon, esprocarb, ethalfluralin, ethametsulfuron, ethidimuron, ethiolate, ethofumesate, ethoxyfen, ethoxysulfuron, etinofen, etnipromid, etobenzanid, EXD, fenasulam, fenoprop, fenoxaprop, fenoxaprop-P, fenoxasulfone, fenteracol, fenthiaprop, fentrazamide, fenuron, ferrous sulfate, flamprop, flamprop-M, flazasulfuron, florasulam, fluazifop, fluazifop-P, fluazolate, flucarbazone, flucetosulfuron, fluchloralin, flufenacet, flufenican, flufenpyr, flumetsulam, flumezin, flumiclorac, flumioxazin, flumipropyn, fluometuron, fluorodifen, fluoroglycofen, fluoromidine, fluoronitrofen, fluothiuron, flupoxam, flupropacil, flupropanate, flupyrsulfuron, fluridone, flurochloridone, fluroxypyr, flurtamone, fluthiacet, fomesafen, foramsulfuron, fosamine, furyloxyfen, glufosinate, glufosinate-P, glyphosate, halauxifen, halosafen, halosulfuron, haloxydine, haloxyfop, haloxyfop-P, hexachloroacetone, hexaflurate, hexazinone, imazamethabenz, imazamox, imazapic, imazapyr, imazaquin, imazethapyr, imazosulfuron, indanofan, indaziflam, iodobonil, iodomethane, iodosulfuron, ioxynil, ipazine, ipfencarbazone, iprymidam, isocarbamid, isocil, isomethiozin, isonoruron, isopolinate, isopropalin, isoproturon, isouron, isoxaben, isoxachlortole, isoxaflutole, isoxapyrifop, karbutilate, ketospiradox, lactofen, lenacil, linuron, MAA, MAMA, MCPA, MCPA-thioethyl, MCPB, mecoprop, mecoprop-P, medinoterb, mefenacet, mefluidide, mesoprazine, mesosulfuron, mesotrione, metam, metamifop, metamitron, metazachlor, metazosulfuron, metflurazon, methabenzthiazuron, methalpropalin, methazole, methiobencarb, methiozolin, methiuron, methometon, methoprotryne, methyl bromide, methyl isothiocyanate, methyldymron, metobenzuron, metobromuron, metolachlor, metosulam, metoxuron, metribuzin, metsulfuron, molinate, monalide, monisouron, monochloroacetic acid, monolinuron, monuron, morfamquat, MSMA, naproanilide, napropamide, naptalam, neburon, nicosulfuron, nipyraclofen, nitralin, nitrofen, nitrofluorfen, norflurazon, noruron, OCH, orbencarb, ortho-dichlorobenzene, orthosulfamuron, oryzalin, oxadiargyl, oxadiazon, oxapyrazon, oxasulfuron, oxaziclomefone, oxyfluorfen, parafluron, paraquat, pebulate, pelargonic acid, pendimethalin, penoxsulam, pentachlorophenol, pentanochlor, pentoxazone, perfluidone, pethoxamid, phenisopham, phenmedipham, phenmedipham-ethyl, phenobenzuron, phenylmercury acetate, picloram, picolinafen, pinoxaden, piperophos, potassium arsenite, potassium azide, potassium cyanate, pretilachlor, primisulfuron, procyazine, prodiamine, profluazol, profluralin, profoxydim, proglinazine, prometon, prometryn, propachlor, propanil, propaquizafop, propazine, propham, propisochlor, propoxycarbazone, propyrisulfuron, propyzamide, prosulfalin, prosulfocarb, prosulfuron, proxan, prynachlor, pydanon, pyraclonil, pyraflufen, pyrasulfotole, pyrazolynate, pyrazosulfuron, pyrazoxyfen, pyribenzoxim, pyributicarb, pyriclor, pyridafol, pyridate, pyriftalid, pyriminobac, pyrimisulfan, pyrithiobac, pyroxasulfone, pyroxsulam, quinclorac, quinmerac, quinoclamine, quinonamid, quizalofop, quizalofop-P, rhodethanil, rimsulfuron, saflufenacil, S-metolachlor, sebuthylazine, secbumeton, sethoxydim, siduron, simazine, simeton, simetryn, SMA, sodium arsenite, sodium azide, sodium chlorate, sulcotrione, sulfallate, sulfentrazone, sulfometuron, sulfosulfuron, sulfuric acid, sulglycapin, swep, TCA, tebutam, tebuthiuron, tefuryltrione, tembotrione, tepraloxydim, terbacil, terbucarb, terbuchlor, terbumeton, terbuthylazine, terbutryn, tetrafluron, thenylchlor, thiazafluron, thiazopyr, thidiazimin, thidiazuron, thiencarbazone-methyl, thifensulfuron, thiobencarb, tiocarbazil, tioclorim, topramezone, tralkoxydim, triafamone, tri-allate, triasulfuron, triaziflam, tribenuron, tricamba, triclopyr, tridiphane, trietazine, trifloxysulfuron, trifluralin, triflusulfuron, trifop, trifopsime, trihydroxytriazine, trimeturon, tripropindan, tritac, tritosulfuron, vernolate, and xylachlor.

Another embodiment of the present disclosure is a method for the control or prevention of fungal attack. This method comprises applying to the soil, plant, roots, foliage, or locus of the fungus, or to a locus in which the infestation is to be prevented (for example applying to cereal or grape plants), a fungicidally effective amount of one or more of the compounds of Formula I. The compounds are suitable for treatment of various plants at fungicidal levels, while exhibiting low phytotoxicity. The compounds may be useful both in a protectant and/or an eradicant fashion.

The compounds have been found to have significant fungicidal effect particularly for agricultural use. Many of the compounds are particularly effective for use with agricultural crops and horticultural plants.

It will be understood by those skilled in the art that the efficacy of the compound for the foregoing fungi establishes the general utility of the compounds as fungicides.

The compounds have broad ranges of activity against fungal pathogens. Exemplary pathogens may include, but are not limited to, causing agent of wheat leaf blotch (Mycosphaerella graminicola; anamorph: Zymoseptoria tritici), wheat brown rust (Puccinia triticina), wheat stripe rust (Puccinia striiformis), scab of apple (Venturia inaequalis), powdery mildew of grapevine (Uncinula necator), barley scald (Rhynchosporium secalis), blast of rice (Magnaporthe grisea), rust of soybean (Phakopsora pachyrhizi), glume blotch of wheat (Leptosphaeria nodorum), powdery mildew of wheat (Blumeria graminis f. sp. tritici), powdery mildew of barley (Blumeria graminis f sp. hordei), powdery mildew of cucurbits (Erysiphe cichoracearum), anthracnose of cucurbits (Glomerella lagenarium), leaf spot of beet (Cercospora beticola), early blight of tomato (Alternaria solani), and spot blotch of barley (Cochliobolus sativus). The exact amount of the active material to be applied is dependent not only on the specific active material being applied, but also on the particular action desired, the fungal species to be controlled, and the stage of growth thereof, as well as the part of the plant or other product to be contacted with the compound. Thus, all the compounds, and formulations containing the same, may not be equally effective at similar concentrations or against the same fungal species.

The compounds are effective in use with plants in a disease-inhibiting and phytologically acceptable amount. The term “disease-inhibiting and phytologically acceptable amount” refers to an amount of a compound that kills or inhibits the plant disease for which control is desired, but is not significantly toxic to the plant. This amount will generally be from about 0.1 to about 1000 ppm (parts per million), with 1 to 500 ppm being preferred. The exact concentration of compound required varies with the fungal disease to be controlled, the type of formulation employed, the method of application, the particular plant species, climate conditions, and the like. A suitable application rate is typically in the range from about 0.10 to about 4 pounds/acre (about 0.01 to 0.45 grams per square meter, g/m²).

Any range or desired value given herein may be extended or altered without losing the effects sought, as is apparent to the skilled person for an understanding of the teachings herein.

The compounds of Formula I may be made using well-known chemical procedures. Intermediates not specifically mentioned in this disclosure are either commercially available, may be made by routes disclosed in the chemical literature, or may be readily synthesized from commercial starting materials utilizing standard procedures.

### General Schemes

The following schemes illustrate approaches to generating picolinamide compounds of Formula (I). The following descriptions and examples are provided for illustrative purposes and should not be construed as limiting in terms of substituents or substitution patterns.

The compound of Formula 1.5 can be prepared as outlined in Scheme 1, steps a-e. The morpholino amide of Formula 1.1 can be prepared by heating the compound of Formula 1.0 and morpholine at an elevated temperature of about 95° C., as shown in a. The compound of Formula 1.1 can be treated with a base, such as sodium hydride (NaH), and 4-methoxybenzylchloride in a polar, aprotic solvent like N,N-dimethylformamide (DMF) at a temperature from about 0° C. to about 22° C. to provide the compound of Formula 1.2, as shown in b. The compound of Formula 1.2 can be treated with a Grignard reagent, such as 1-propenylmagnesium bromide, in a polar, aprotic solvent like tetrahydrofuran (THF) at a reduced temperature of about 0° C., as shown in c, to provide the compound of Formula 1.3. The compound of Formula 1.3 can be added as a solution in a polar, aprotic solvent like THF to a solution of zinc borohydride, prepared from zinc chloride (ZnCl₂) and sodium borohydride (NaBH₄) in diethyl ether (Et₂O), at a temperature from about 0° C. to about 22° C. to afford the compound of Formula 1.4, as shown in d. The compound of Formula 1.4 can be treated with a base, such as NaH, and a benzylic halide, such as benzylbromide, in a polar, aprotic solvent like DMF at a temperature from about 0° C. to about 22° C. to provide the compound of Formula 1.5, as shown in e.

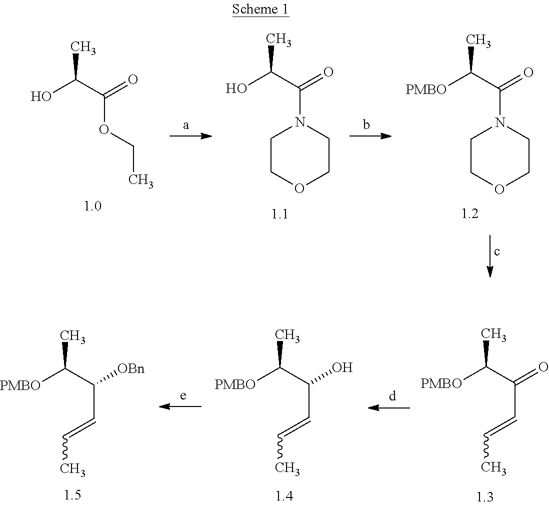

The compound of Formula 2.1 can be obtained using the method outlined in Scheme 2, steps a-b. The compound of Formula 1.5 can be subjected to ozonolysis conditions, such as treating with ozone (03) in a solvent mixture such as dichloromethane (DCM) and methanol (MeOH) at a temperature of about −78° C., and then quenching with a reducing agent, such as NaBH₄, to provide the compound of Formula 2.0, as shown in a. The compound of Formula 2.1 can be obtained by treating an alcohol of Formula 2.0 with a base, such as NaH, and an allylic halide, such as allylbromide, in a polar, aprotic solvent like DMF at a temperature from about 0° C. to about 22° C., as shown in b.

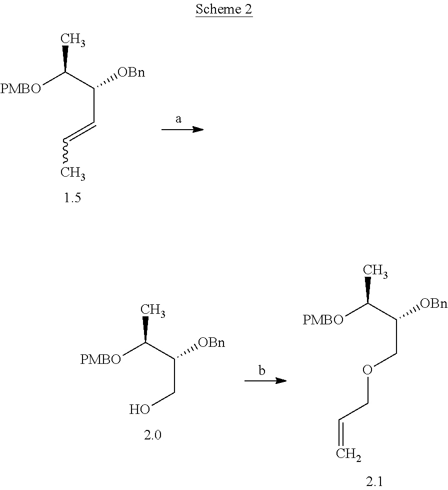

Compounds of Formula 3.3, wherein R₈ is as originally defined, can be prepared by the method shown in Scheme 3, steps a-b. Compounds of Formula 3.1, wherein R₈ is as originally defined and R₉ is alkyl or alkoxy, can be prepared from compounds of Formula 3.0, wherein R₈ is as originally defined, by treatment with an alkoxy borane, such as pinacol borane, in the presence of a nickel catalyst, such as bis(cyclooctadiene)nickel(0) (Ni(cod)₂), as described by Ely, R. J.; Morken, J. P. J. Am. Chem. Soc. 2010, 132, 2534-2535, in a solvent such as toluene and at a temperature of about 0° C. to 22° C., as shown in a. Compounds of Formula 3.3, wherein R₈ is as previously defined, can be prepared from compounds of Formula 3.1, wherein R₈ and R₉ are as previously defined, by treatment with a benzyl (Bn) protected lactate-derived aldehyde, such as the compound of Formula 3.2, prepared and characterized as described in Cheng, C.; Brookhart, M. Angew. Chem. Int. Ed. 2012, 51, 9422-9424 and Takai, K.; Heathcock, C. H. J. Org. Chem. 1985, 50, 3247-3251, respectively, as shown in b.

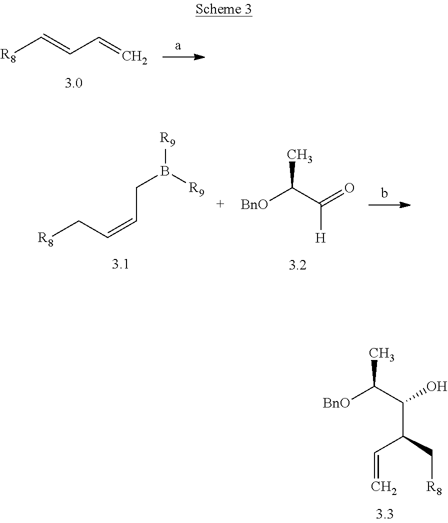

Compounds of Formula 4.3, wherein R₂ is as originally defined, but is not hydrogen, can be prepared according to the method shown in Scheme 4, steps a-c. Compounds of Formula 3.3, wherein R₂ is as originally defined, but is not hydrogen, can be treated with a palladium catalyst, such as palladium dichloride (PdCl₂), and a copper salt, such as copper chloride (CuCl), in a solvent mixture such as DMF and water (H₂O) under an oxygen (O₂) atmosphere at an elevated temperature of about 65° C. to afford compounds of Formula 4.0, wherein R₂ is as originally defined, but is not hydrogen, as shown in a. An inconsequential mixture of compounds of Formula 4.1 and 4.2, wherein R₂ is as originally defined, but is not hydrogen, can be obtained by treating compounds of Formula 4.0, wherein R₂ is as previously defined, with peroxybis(trimethylsilane) and trimethylsilyl trifluromethansulfonate in a halogenated solvent, such as DCM, at a reduced temperature from about −15° C. to about −10° C., as shown in b. A mixture of compounds of Formula 4.1 and 4.2, wherein R₂ is as previously defined, can be treated with a base, such as potassium carbonate (K₂CO₃), in a solvent mixture such as aqueous MeOH to afford diols of Formula 4.3, wherein R₂ is as previously defined, as depicted in c.

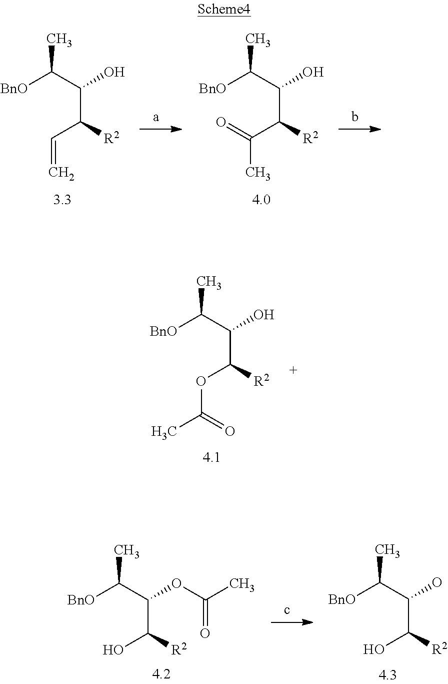

Compounds of Formula 5.1, wherein R₂ is as originally defined, but is not hydrogen, and compounds of Formula 5.2, wherein R₁ is alkyl and R₂ is as originally defined, but is not hydrogen, can be prepared according to the method shown in Scheme 5, steps a-c. Compounds of Formula 4.2, wherein R₂ is as originally defined, but is not hydrogen, can be treated with an allylic halide, such as allyl bromide, potassium iodide (KI), and a base, such as potassium carbonate (K₂CO₃), in the presence of a catalyst, such as 2,2-diphenyl-1,3,2-oxazaborolidin-3-ium-2-uide, in a polar, aprotic solvent like acetonitrile (CH₃CN) at an elevated temperature of about 60° C. to afford compounds of Formula 5.0, wherein R₂ is as originally defined, but is not hydrogen, as depicted in a and described by Lee, D.; Williamson, C. L.; Chan, L.; Taylor, M. S. J. Am. Chem. Soc. 2012, 134, 8260-8267. Compounds of Formula 5.1, wherein R₂ is as previously defined, can be prepared by treating compounds of Formula 5.0, wherein R₂ is as previously defined, with triisopropylsilyl trifluoromethanesulfonate and an amine base, such as 2,6-lutidine, in a halogenated solvent like DCM, as shown in b. Alternatively, compounds of Formula 5.2, wherein R₁ and R₂ are as previously defined, can be prepared by treating compounds of Formula 5.0, wherein R₂ is as previously defined, with a base such as potassium tert-butoxide and an alkyl halide like (bromomethyl)cyclopropane in a polar, aprotic solvent like THF or DMF at a temperature from about 22° C. to about 40° C., as shown in c.

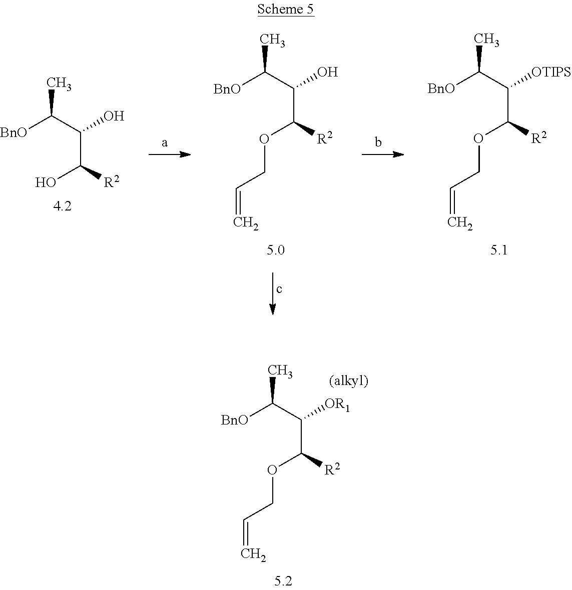

Compounds of Formula 6.3, wherein R₂ is as originally defined, can be obtained by the method outlined in Scheme 6, steps a-c. Compounds of Formula 6.0, wherein R₂ is as originally defined, can be treated with O₃ in a solvent mixture such as DCM and MeOH at a reduced temperature of about −78° C., followed by quenching with a reducing agent, such as triphenylphosphine (PPh₃), as shown in a, to provide aldehydes of Formula 6.1, wherein R₂ is as originally defined. Compounds of Formula 6.1, wherein R₂ is as previously defined, can be treated with an ylide precursor, such as methyl 2-((tert-butoxycarbonyl)amino)-2-(dimethoxyphosphoryl)acetate, and a base, such as 1,8-diazabicyclo[5.4.0]undec-7-ene (DBU), in a halogenated solvent like DCM to afford compounds of Formula 6.2, wherein R₂ is as originally defined, as shown in b. Compounds of Formula 6.2, wherein R₂ is as previously defined, can be treated with a chiral catalyst such as (+)-1,2-bis((2S,5S)-2,5-diethylphospho-lano)benzene(1,5-cyclooctadiene)rhodium(I) trifluoromethanesulfonate ((S,S)-Et-DuPhos-Rh) in the presence of hydrogen gas (H₂) to afford compounds of Formula 6.3, wherein R₂ is as previously defined, as shown in c.

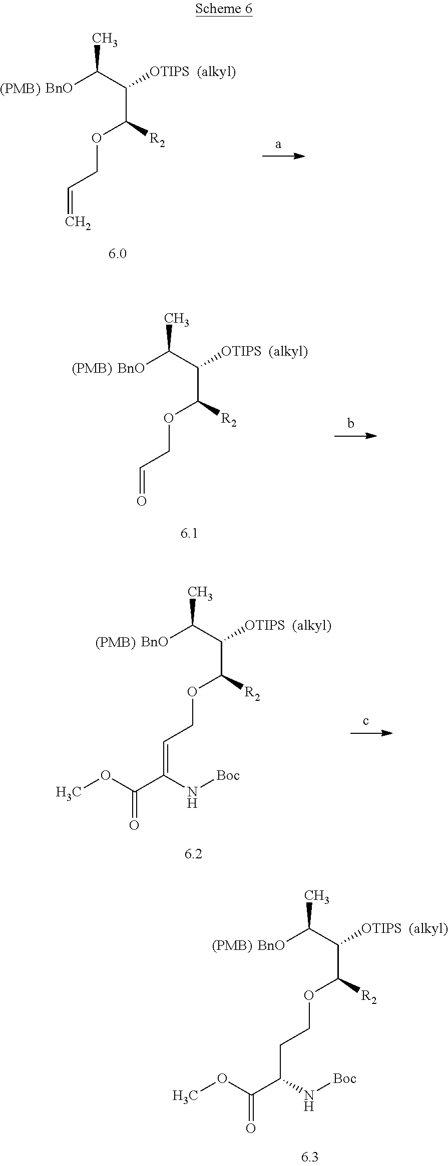

Compounds of Formula 7.2, wherein R₂ is as originally defined, but is not hydrogen, can be prepared as outlined in Scheme 7, steps a-b. Treating compounds of Formula 7.0, wherein R₂ is as originally defined, but is not hydrogen, with a hydroxide base, such as lithium hydroxide monohydrate (LiOH—H₂O) in a solvent mixture such as aqueous THF, as depicted in a, provides compounds of Formula 7.1, wherein R₂ is as originally defined, but is not hydrogen. Treating compounds of Formula 7.1, wherein R₂ is as previously defined, with a catalyst, such as palladium on carbon (Pd/C), in the presence of H₂ in a solvent such as ethyl acetate (EtOAc), as shown in b, affords compounds of Formula 7.2, wherein R₂ is as previously defined.

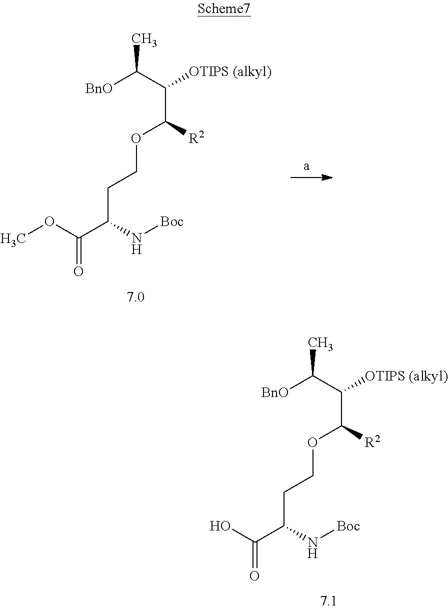

The compound of Formula 8.2 can be obtained from the compound of Formula 8.0, as depicted in Scheme 8, steps a-b. The compound of Formula 8.1 can be obtained from a compound of Formula 8.0 by treatment with an oxidant, such as 2,3-dichloro-5,6-dicyano-p-benzoquinone (DDQ), in a solvent mixture like aqueous DCM, as shown in a. Treating the compound of Formula 8.1 with a hydroxide base such as LiOH—H₂O in a solvent mixture such as aqueous THF, as depicted in b, provides the compound of Formula 8.2.

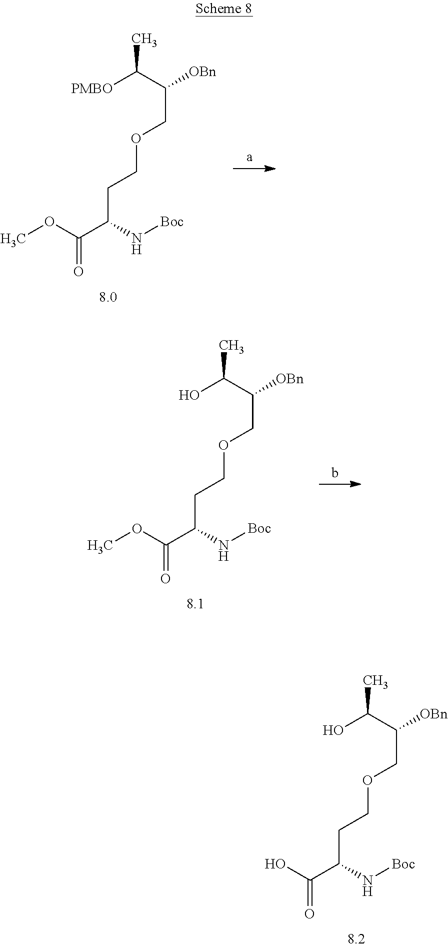

Compounds of Formula 9.1, wherein R₂ is as originally defined, can be prepared according to the method outlined in Scheme 9. Compounds of Formula 9.1, wherein R₂ is as previously defined, can be obtained from compounds of Formula 9.0, wherein R₂ is as originally defined, by the addition of a solution of compounds of Formula 9.0 in a halogenated solvent, such as DCM, or an aromatic solvent, such as toluene, to a mixture of a base, such as 4-dimethylaminopyridine (DMAP), and a mixed anhydride, such as 2-methyl-6-nitrobenzoic anhydride (MNBA), in either a halogenated solvent like DCM or an aromatic solvent like toluene over a period of 4-12 hours (h), as shown in a.

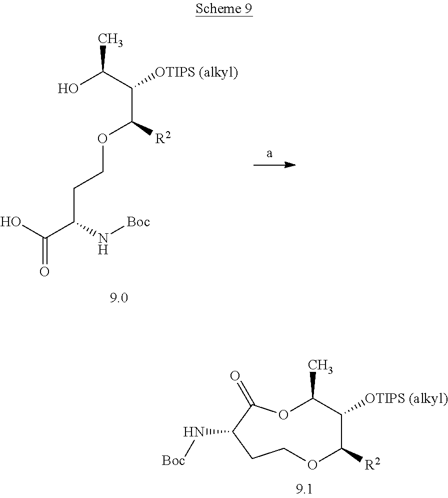

Compounds of Formula 10.1, wherein R₂ is as originally defined, but is not hydrogen, can be prepared according to the method outlined in Scheme 10. Compounds of Formula 10.1, wherein R₂ is as previously defined, can be obtained from compounds of Formula 10.0, wherein R₂ is as originally defined, but is not hydrogen, by exposure to a fluoride source, such as tetra-N-butylammonium fluoride (TBAF), in a polar, aprotic solvent like THF at a reduced temperature of about 0° C., as shown in a.

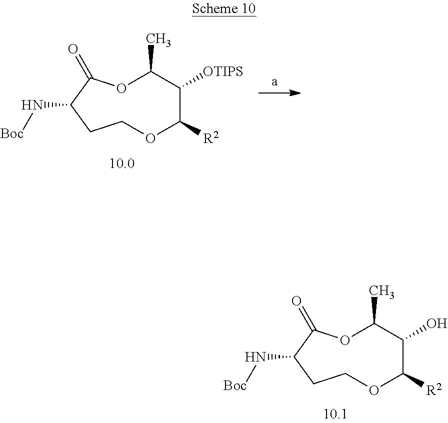

The compound of Formula 11.1 can be prepared according to the method outlined in Scheme 11. The compound of Formula 11.0 can be treated with a catalyst, such as Pd/C, in the presence of H₂ in a solvent such as EtOAc to afford the compound of Formula 11.1, as shown in a.

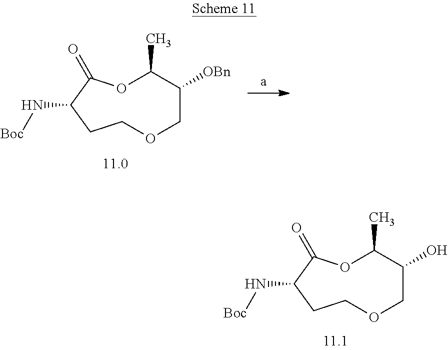 [SMILES: C[C@@H]1OC(=O)[C@@H](NC(=O)OC(C)(C)C)CCOC[C@H]1OCc1ccccc1 ; C[C@@H]1OC(=O)[C@@H](NC(=O)OC(C)(C)C)CCOC[C@H]1O]

Compounds of Formulae 12.1, 12.2, 12.3, and 12.4, wherein R₁ and R₂ are as originally defined, can be obtained using the methods outlined in Scheme 12, steps a-d. Compounds of Formula 12.1, wherein R₁ is acyl and R₂ is as previously defined, can be prepared from compounds of Formula 12.0, wherein R₂ is as originally defined, by treatment with an acyl halide, such as isobutyryl chloride, in the presence of a base, such as triethylamine (NEt₃) and an amine catalyst, such as DMAP, in a halogenated solvent like DCM, as shown in a. Alternatively, as shown in b, compounds of Formula 12.2, wherein R₁ is aryl and R₂ is as previously defined, can be prepared from compounds of Formula 12.0, wherein R₂ is as previously defined, by treatment with an organometallic species, such as bis(acetato-O)triphenylbismuth(V) (Bi(OAc)₂Ph₃), in the presence of a catalyst, such as copper(II) acetate (Cu(OAc)₂), in an aromatic hydrocarbon solvent like toluene at an elevated temperature of about 50° C. Compounds of Formula 12.3, wherein R₁ is alkenyl and R₂ is as originally defined, can be prepared from compounds of Formula 12.0, wherein R₂ is as previously defined, by treatment with an allyl carbonate, such as tert-butyl (2-methylallyl) carbonate, in the presence of a catalyst, such as tris(dibenzylideneacetone)-dipalladium(0) (Pd₂(dba)₃), and a ligand, such as 1,1′-bis(diphenylphosphino)ferrocene (dppf), in an aprotic solvent like THF or toluene at an elevated temperature of about 60° C. Additionally, compounds of Formula 12.4, wherein R₁ is alkyl and R₂ is as previously defined, can be prepared by treating compounds of Formula 12.3, wherein R₁ and R₂ are as previously defined, with a catalyst such as Pd/C in the presence of H₂ in a solvent like EtOAc, as shown in d.

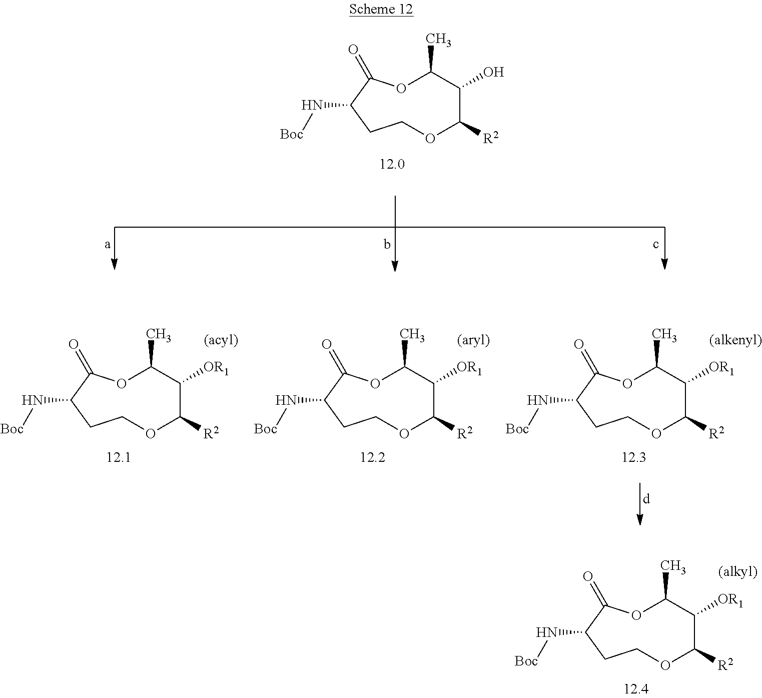

Compounds of Formula 13.3, wherein R₁ and R₂ are as originally defined, but R₁ is not silyl or hydrogen, can be prepared through the methods shown in Scheme 13, steps a-d. Compounds of Formula 13.0, wherein R₁ and R₂ are as originally defined, but R₁ is not alkenyl, silyl, or hydrogen, can be treated with an acid such as a 4 N solution of hydrogen chloride (HCl) in dioxane in a halogenated solvent like DCM to afford compounds of Formula 13.1, wherein R₁ and R₂ are as originally defined, but R₁ is not alkenyl, silyl, or hydrogen, as depicted in a. The resulting hydrochloride salt may be neutralized prior to use to give the free amine or neutralized in situ in step b. Compounds of Formula 13.3, wherein R₁ and R₂ are as originally defined, but R₁ is not alkenyl, silyl, or hydrogen, can be prepared from compounds of Formula 13.1, wherein R₁ and R₂ are as previously defined, by treating with 3-hydroxy-4-methoxypicolinic acid in the presence of a base, such as diisopropylethylamine, and a peptide coupling reagent, such as benzotriazol-1-yl-oxytripyrrolidinophosphonium hexafluorophosphate (PyBOP) or O-(7-azabenzo-triazol-1-yl)-N,N,N′,N′-tetramethyluronium hexafluorophosphate (HATU), in an halogenated solvent like DCM, as shown in b. Alternatively, compounds of Formula 13.0, wherein R₁ is alkenyl and R₂ is as originally defined, can be treated with trimethylsilyl trifluoromethanesulfonate and a base, such as 2,6-lutidine, in a halogenated solvent like DCM, followed by treatment with an alcohol, such as MeOH, to afford compounds of Formula 13.2, wherein R₁ is alkenyl and R₂ is as originally defined, as depicted in c. Compounds of Formula 13.3, wherein R₁ is alkenyl and R₂ is as originally defined, can be prepared from compounds of Formula 13.2, wherein R₁ and R₂ are as previously defined, by treating with 3-hydroxy-4-methoxypicolinic acid in the presence of a base, such as diisopropylethylamine, and a peptide coupling reagent, such as PyBOP or HATU, in an halogenated solvent like DCM, as shown in d.

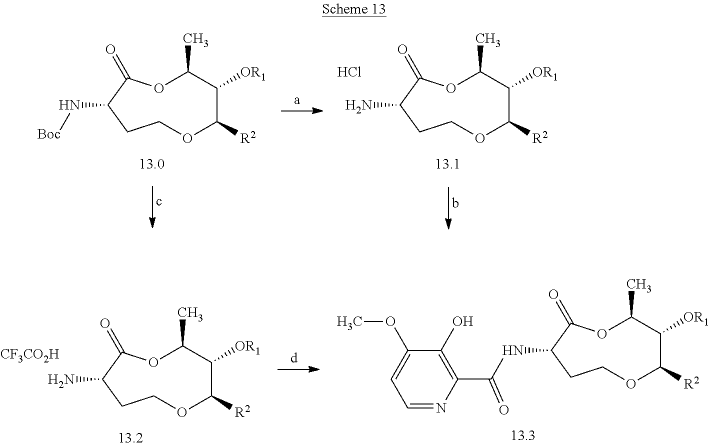

Compounds of Formula 14.0, wherein R₁, R₂, and R₄ are as originally defined, but R₁ is not silyl or hydrogen, can be prepared according to the method outlined in Scheme 14. Compounds of Formula 14.0, wherein R₁, R₂, and R₄ are as previously defined, can be prepared from compounds of Formula 13.3, wherein R₁ and R₂ are as originally defined, but R₁ is not silyl or hydrogen, by treating with an appropriate alkyl halide with or without a reagent such as sodium iodide (NaI) and an alkali carbonate base, such as sodium carbonate (Na₂CO₃) or potassium carbonate (K₂CO₃), in a solvent like acetone or by treatment with an acyl halide in the presence of an amine base, such as pyridine, NEt₃, DMAP, or mixtures thereof, in an aprotic solvent such as DCM.

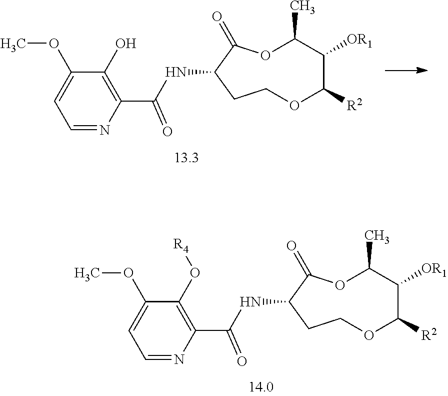

### EXAMPLES

### Example 1, Step 1: Preparation of (S)-2-hydroxy-1-morpholinopropan-1-one

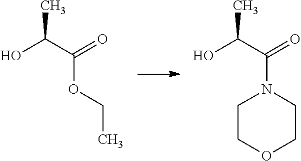

A solution of (S)-ethyl 2-hydroxypropanoate (10.0 grams (g), 85.0 millimoles (mmol)) and morpholine (15.1 milliliters (mL), 174 mmol) was stirred at 95° C. for 72 hours (h). The reaction mixture was cooled to room temperature and the excess morpholine was evaporated under reduced pressure (1 torr, 50° C.). The resulting crude residue was purified by flash column chromatography (silica gel (SiO₂), 0→90% acetone in hexanes) to afford the title compound (12.0 g, 89%) as a yellow oil: ¹H NMR (400 MHz, CDCl₃) δ 4.44 (dq, J=7.4, 6.7 Hz, 1H), 3.81-3.58 (m, 7H), 3.46-3.38 (m, 2H), 1.33 (d, J=6.6 Hz, 3H); ESIMS m/z 160 ([M+H]⁺).

### Example 1, Step 2: Preparation of (S)-2-((4-methoxybenzyl)oxy)-1-morpholinopropan-1-one*

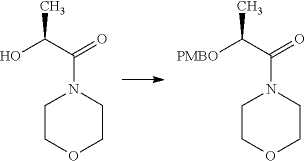

A solution of (S)-2-hydroxy-1-morpholinopropan-1-one (8.00 g, 50.3 mmol) in tetrahydrofuran (THF; 26 mL) was added to a suspension of sodium hydride (NaH; 3.02 g, 75 mmol, 60% dispersion in mineral oil) in N,N-dimethylformamide (DMF; 100 mL) at 0° C. After stirring for 5 minutes (min) at 0° C., neat 1-(chloromethyl)-4-methoxybenzene (8.18 mL, 60.3 mmol) was added slowly. The reaction mixture was removed from the cold bath and stirred at room temperature for 5 h. The reaction mixture was quenched with 1/2 saturated (sat.) aqueous (aq.) ammonium chloride (NH₄Cl; 100 mL) and diluted with diethyl ether (Et₂O; 100 mL). The phases were separated and the aq. phase was extracted with Et₂O (2×100 mL), and the combined organic phases were washed with sat. aq. sodium chloride (NaCl, brine; 50 mL), dried over sodium sulfate (Na₂SO₄), filtered, and evaporated. The resulting crude residue was purified by flash column chromatography (SiO₂, 0→460% acetone in hexanes) to afford the title compound (9.85 g, 70%) as a colorless oil: ¹H NMR (500 MHz, CDCl₃) δ 7.28-7.21 (m, 2H), 6.92-6.85 (m, 2H), 4.52 (d, J=11.2 Hz, 1H), 4.41 (d, J=11.3 Hz, 1H), 4.30 (q, J=6.8 Hz, 1H), 3.81 (s, 3H), 3.73-3.56 (m, 8H), 1.43 (d, J=6.8 Hz, 3H); ¹³C NMR (126 MHz, CDCl₃) δ 170.68, 159.43, 129.60, 129.46, 113.88, 75.11, 70.89, 67.06, 66.80, 55.30, 45.64, 42.50, 17.84; ESIMS m/z 280 ([M+H]⁺). *Reference: Nogawa, M.; Sugawara, S.; Iizuka, R.; Shimojo, M.; Ohta, H.; Hatanaka, M.; Matsumoto, K. Tetrahedron 2006, 62, 12071-12083.

### Example 1, Step 3: Preparation of (S)-2-((4-methoxybenzyl)oxy)hex-4-en-3-one

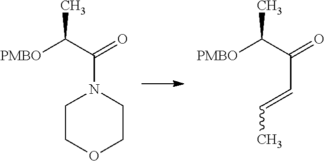

To a solution of (S)-2-((4-methoxybenzyl)oxy)-1-morpholinopropan-1-one (5.00 g, 17.9 mmol) in THF (36 mL) was added prop-1-en-1-ylmagnesium bromide (53.6 mL, 26.8 mmol, 0.5 Molar (M) in THF) over a 15 min period at 0° C. The resulting mixture was stirred at 0° C. for 4 h and then poured into a well-stirred, 0° C. solution of 1 M sodium hydrogen sulfate (NaHSO₄; 100 mL). The mixture was partitioned between water (H₂O; 25 mL) and Et₂O (75 mL) and the phases were separated. The aq. phase was extracted with Et₂O (2×75 mL) and the combined organic phases were washed with brine (30 mL), dried over Na₂SO₄, filtered, and evaporated. The crude residue was purified by flash column chromatography (SiO₂, 0→425% acetone in hexanes) to afford an approximate 1:1 mixture of E and Z enones of the title compound (3.45 g, 82%) as a colorless oil: ¹H NMR (400 MHz, CDCl₃) δ 7.30-7.22 (m, 4H), 7.06 (dq, J=15.6, 6.9 Hz, 1H), 6.92-6.84 (m, 4H), 6.59-6.48 (m, 2H), 6.38 (dq, J=11.5, 7.2 Hz, 1H), 4.50 (dd, J=11.2, 3.1 Hz, 2H), 4.37 (dd, J=11.2, 4.9 Hz, 2H), 4.02 (q, J=6.9 Hz, 1H), 3.91 (q, J=6.9 Hz, 1H), 3.83-3.78 (m, 6H), 2.17 (dd, J=7.2, 1.7 Hz, 3H), 1.92 (dd, J=6.9, 1.7 Hz, 3H), 1.34 (d, J=4.6 Hz, 3H), 1.32 (d, J=4.6 Hz, 3H); ¹³C NMR (101 MHz, CDCl₃) δ 203.45, 201.26, 159.38, 159.36, 146.04, 144.53, 129.81, 129.75, 129.56, 129.53, 126.12, 122.89, 113.86, 80.81, 79.70, 71.51, 71.46, 55.29, 18.54, 18.00, 17.82, 16.24; ESIMS m/z 257 ([M+Na]⁺).

### Example 1, Step 4: Preparation of (2S,3R)-2-((4-methoxybenzyl)oxy)hex-4-en-3-ol*

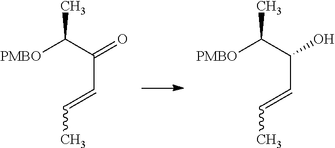

To a solution of sodium borohydride (NaBH₄; 1.32 g, 34.8 mmol) in Et₂O (29 mL) was added zinc(II) chloride (ZnCl₂; 18.1 mL, 18.1 mmol, 1 M in Et₂O) over a 15 min period at 0° C., and the reaction mixture was allowed to warm to room temperature overnight as the ice melted. The resulting solution of zinc borohydride (Zn(BH₄)₂) was cooled to 0° C. and treated with a solution of (S)-2-((4-methoxybenzyl)oxy)hex-4-en-3-one (3.4 g, 14.5 mmol) in THF (29 mL) over a 15 min period via canular transfer (rinsing with 4 mL THF). The reaction mixture was stirred at 0° C. for 2 h, removed from the cold bath, and then stirred at room temperature for 30 min. The reaction mixture was diluted with Et₂O (50 mL) and carefully quenched by the sequential addition of 1/2 sat. aq. NH₄Cl (40 mL) and H₂O (40 mL). The phases were separated and the aq. phase was extracted with Et₂O (2×50 mL). The combined organic phases were washed with brine (25 mL), dried over Na₂SO₄, filtered, and evaporated. The crude residue was purified by flash column chromatography (SiO₂, 0→30% acetone in hexanes) to afford an approximate 1:1 mixture of E and Z olefin isomers of the title compound with a 10:1 ratio of diastereomers (2.95 g, 86%) as a colorless oil: ¹H NMR (400 MHz, CDCl₃) major alcohol diastereomer δ 7.31-7.23 (m, 2H), 6.93-6.84 (m, 2H), 5.79-5.58 (m, 1H), 5.54-5.38 (m, 1H), 4.66-4.51 (m, 1.5H), 4.50-4.42 (m, 1H), 4.19-4.08 (m, 0.5H), 3.80 (app s, 3H), 3.63-3.50 (m, 1H), 2.18 (s, 1H), 1.75-1.62 (m, 3H), 1.17-1.09 (m, 3H); ¹³C NMR (101 MHz, CDCl₃) δ 159.21, 130.61, 130.59, 129.43, 129.24, 129.22, 129.09, 128.61, 127.54, 113.83, 77.16, 74.55, 70.53, 70.51, 69.30, 55.28, 17.88, 14.18, 14.15, 13.49; ESIMS m/z 236 ([M]⁺). *Reference: Ichikawa, Y.; Egawa, H.; Ito, T.; Isobe, M.; Nakano, K.; Kotsuki, H. Org. Lett. 2006, 8, 5737-5740.

### Example 1, Step 5: Preparation of 1-((((2S,3R)-3-(benzyloxy)hex-4-en-2-yl)oxy)methyl)-4-methoxybenzene

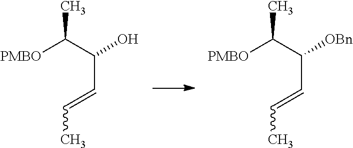

To a solution of (2S,3R)-2-((4-methoxybenzyl)oxy)hex-4-en-3-ol (2.95 g, 12.5 mmol) in DMF (28 mL) was added NaH (0.749 g, 18.7 mmol, 60% dispersion in mineral oil) at 0° C. The reaction mixture was stirred at 0° C. for 10 min and then treated with neat benzyl bromide (1.78 mL, 15.0 mmol). The reaction mixture was stirred for 4 h at 0° C., warmed to room temperature and stirred for 1 h, quenched with 1/2 sat. aq. NH₄Cl (40 mL), and diluted with Et₂O (50 mL). The phases were separated and the aq. phase was extracted with Et₂O (2×50 mL). The combined organic phases were washed with brine (25 mL), dried over Na₂SO₄, filtered and evaporated. The crude residue was purified by flash column chromatography (SiO₂, 0→430% EtOAc in hexanes) to afford an approximate 1:1 mixture of E and Z olefin isomers of the title compound (4.0 g, 88%) as a colorless oil: ¹H NMR (400 MHz, CDCl₃) δ 7.35-7.22 (m, 7H), 6.89-6.81 (m, 2H), 5.81 (dqd, J=11.1, 6.9, 1.1 Hz, 0.5H), 5.69 (dqd, J=15.5, 6.4, 0.8 Hz, 0.5H), 5.53-5.41 (m, 1H), 4.67-4.58 (m, 1H), 4.57-4.51 (m, 2H), 4.44-4.34 (m, 1H), 4.17 (ddd, J=9.3, 4.2, 1.1 Hz, 0.5H), 3.82-3.76 (m, 3H), 3.74-3.66 (m, 0.5H), 3.65-3.54 (m, 1H), 1.80-1.73 (m, 1.5H), 1.61 (dd, J=7.0, 1.8 Hz, 1.5H), 1.22-1.14 (m, 3H); ¹³C NMR (101 MHz, CDCl₃) δ 159.00, 158.98, 139.00, 138.96, 131.19, 131.17, 130.51, 129.23, 129.16, 128.71, 128.57, 128.23, 127.66, 127.58, 127.31, 127.25, 113.64, 83.17, 76.84, 76.64, 71.06, 71.04, 69.97, 55.28, 17.94, 16.47, 16.31, 13.62; ESIMS m/z 349 ([M+Na]⁺).

### Example 2, Step 1: Preparation of (2R,3S)-2-(benzyloxy)-3-((4-methoxybenzyl)oxy)butan-1-ol

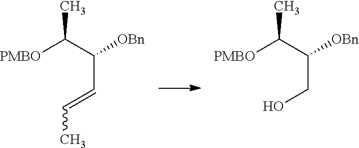

To a solution of 1-((((2S,3R)-3-(benzyloxy)hex-4-en-2-yl)oxy)methyl)-4-methoxybenzene (4.0 g, 12.3 mmol) in dichloromethane (DCM; 28 mL) and methanol (MeOH; 2.8 mL) was added a 0.1% solution of 1-((E)-(4-((E)-phenyldiazenyl)phenyl)diazenyl)-naphthalen-2-ol (Sudan III indicator; 75 microliters (μL)) in DCM. The reaction mixture was cooled to −78° C. (dry ice/acetone) and the flask was connected to an ozone generator. Ozone (O₃) was bubbled through the solution until the solution became colorless (˜30 min). Oxygen gas (O₂) was then bubbled through the solution for 5 min and the solution was treated with MeOH (6 mL) and NaBH₄ (1.39 g, 36.8 mmol). The flask was removed from the cold bath and the reaction mixture was allowed to slowly warm to room temperature overnight. The reaction mixture was quenched with 1/2 sat. aq. NH₄Cl (40 mL) and diluted with DCM (40 mL) and H₂O (20 mL). The phases were separated and the aq. phase was extracted with DCM (2×40 mL). The combined organic phases were washed with brine (15 mL), dried by passing through a phase separator cartridge, and evaporated. The resulting crude residue was purified by flash column chromatography (SiO₂, 0→15% acetone in hexanes) to afford the title compound (3.18 g, 82%) as a colorless oil: ¹H NMR (400 MHz, CDCl₃) δ 7.37-7.21 (m, 7H), 6.91-6.82 (m, 2H), 4.63 (d, J=1.3 Hz, 2H), 4.57 (d, J=11.3 Hz, 1H), 4.45 (d, J=11.3 Hz, 1H), 3.82-3.65 (m, 6H), 3.42 (dt, J=5.7, 4.6 Hz, 1H), 2.33 (t, J=6.2 Hz, 1H), 1.26 (d, J=6.3 Hz, 3H); ¹³C NMR (101 MHz, CDCl₃) δ 159.25, 138.24, 130.39, 129.37, 128.47, 127.90, 127.81, 113.87, 82.13, 75.26, 72.45, 70.93, 61.80, 55.29, 16.51; ESIMS m/z 339 ([M+Na]⁺).

### Example 2, Step 2: Preparation of 1-((((2S,3R)-4-(allyloxy)-3-(benzyloxy)butan-2-yl)oxy)methyl)-4-methoxybenzene

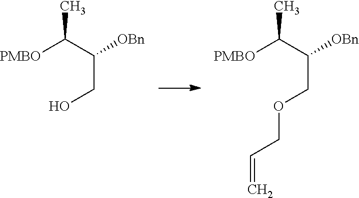

To a solution of (2R,3S)-2-(benzyloxy)-3-((4-methoxybenzyl)oxy)butan-1-ol (3.18 g, 10.1 mmol) in DMF (25 mL) was added NaH (0.603 g, 15.1 mmol, 60% dispersion in mineral oil) at 0° C. The reaction mixture was stirred at 0° C. for 10 min and treated with neat allyl bromide (0.870 mL, 10.1 mmol). After 3 h at 0° C., the reaction mixture was removed from the cold bath, stirred for an additional 1 h, quenched with 1/2 sat. aq. NH₄Cl (40 mL), and diluted with Et₂O (50 mL). The phases were separated and the aq. phase was extracted with Et₂O (2×50 mL). The combined organic phases were washed with brine (25 mL), dried over Na₂SO₄, filtered, and evaporated. The resulting crude residue was purified by flash column chromatography (SiO₂, 0→20% EtOAc in hexanes) to afford the title compound (3.14 g, 79%) as a colorless oil: ¹H NMR (400 MHz, CDCl₃) δ 7.40-7.28 (m, 4H), 7.30-7.20 (m, 3H), 6.90-6.81 (m, 2H), 5.90 (ddt, J=17.3, 10.3, 5.5 Hz, 1H), 5.27 (dq, J=17.2, 1.7 Hz, 1H), 5.21-5.13 (m, 1H), 4.74 (d, J=11.8 Hz, 1H), 4.66 (d, J=11.8 Hz, 1H), 4.53 (d, J=11.4 Hz, 1H), 4.45 (d, J=11.4 Hz, 1H), 4.02-3.95 (m, 2H), 3.79 (s, 3H), 3.73-3.54 (m, 4H), 1.23 (d, J=6.3 Hz, 3H); ¹³C NMR (101 MHz, CDCl₃) δ 159.09, 138.86, 134.90, 130.90, 129.22, 128.25, 127.83, 127.44, 116.76, 113.73, 80.92, 74.70, 72.75, 72.28, 70.81, 70.21, 55.28, 16.00; ESIMS m/z 380 ([M+Na]⁺).

### Example 3: Preparation of (2S,3R,4S)-4-benzyl-2-(benzyloxy)hex-5-en-3-ol

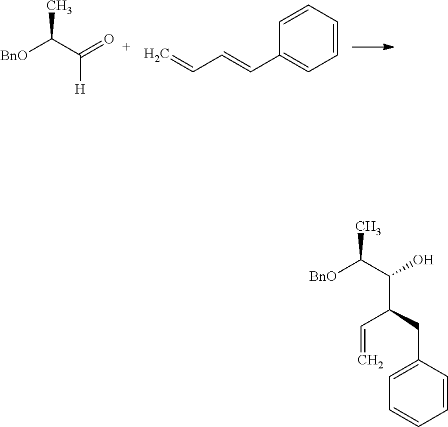

To a round-bottom flask were added bis(cyclooctadiene)nickel(0) (Ni(cod)₂; 0.168 g, 0.609 mmol) and tricyclohexylphosphine (P(C₆H₁₁)₃; 0.213 g, 0.761 mmol) under an inert atmosphere (N₂ glove bag), and the flask was capped and removed from the bag. The mixture was diluted with toluene (22 mL) and 4,4,5,5-tetramethyl-1,3,2-dioxaborolane (6.63 mL, 45.7 mmol) was added at room temperature. The reaction mixture was cooled to 0° C. in an ice bath and treated with neat (E)-buta-1,3-dien-1-ylbenzene (4.76 g, 36.5 mmol) dropwise over a 10 min period. The mixture was removed from the ice bath and stirred at room temperature for 2 h, cooled to −78° C. in a dry ice/acetone bath, and treated with (S)-2-(benzyloxy)propanal (5 g, 30.5 mmol) followed by boron trifluoride diethyl etherate (BF₃.OEt₂; 0.376 mL, 3.05 mmol). The reaction mixture was allowed to slowly warm to room temperature overnight, and quenched by treating with MeOH (5 mL). After stirring for 30 min, the reaction mixture was concentrated and purified by flash column chromatography (SiO₂, 0→50% EtOAc in hexanes) to afford the title compound (8.95 g, 99%) as a colorless oil: ¹H NMR (400 MHz, CDCl₃) δ 7.39-7.30 (m, 3H), 7.32-7.25 (m, 1H), 7.25-7.21 (m, 2H), 7.20-7.11 (m, 4H), 5.45 (ddd, J=17.2, 10.3, 9.5 Hz, 1H), 4.93 (dd, J=10.3, 1.8 Hz, 1H), 4.79 (ddd, J=17.2, 1.9, 0.7 Hz, 1H), 4.55 (d, J=11.7 Hz, 1H), 4.46 (d, J=11.7 Hz, 1H), 3.76 (ddd, J=9.2, 3.2, 2.2 Hz, 1H), 3.56 (qd, J=6.3, 3.1 Hz, 1H), 3.19 (dd, J=13.3, 3.5 Hz, 1H), 2.58 (dd, J=13.4, 9.3 Hz, 1H), 2.39 (dt, J=9.2, 3.4 Hz, 1H), 2.37 (d, J=2.3 Hz, 1H), 1.17 (d, J=6.3 Hz, 3H); ¹³C NMR (101 MHz, CDCl₃) δ 139.92, 138.48, 137.37, 129.81, 128.46, 127.94, 127.70, 127.66, 125.76, 117.21, 76.22, 73.78, 70.56, 48.44, 37.63, 12.21; ESIMS m/z 319 ([M+Na]⁺).

### Example 4, Step 1: Preparation of (3R,4R,5S)-5-(benzyloxy)-3-(4-fluorobenzyl)-4-hydroxyhexan-2-one

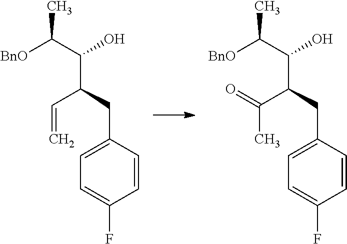 [SMILES: C=C[C@H](Cc1ccc(F)cc1)[C@@H](O)[C@H](C)OCc1ccccc1 ; CC(=O)[C@H](Cc1ccc(F)cc1)[C@@H](O)[C@H](C)OCc1ccccc1]

A solution of (2S,3R,4S)-2-(benzyloxy)-4-(4-fluorobenzyl)hex-5-en-3-ol (1 g, 3.18 mmol) in DMF (13.2 mL) and water (2.6 mL) was sparged with 02 for 5 min and then treated with palladium(II) chloride (PdCl₂; 0.141 g, 0.795 mmol) and copper(I) chloride (CuCl; 0.630 g, 6.36 mmol). The solution was heated to 65° C. and stirred vigorously overnight under a static 02 atmosphere. The reaction mixture was diluted with Et₂O (10 mL) and filtered through Celite®. The filtrate was diluted with additional Et₂O (20 mL) and washed with sat. aq. NH₄Cl. The phases were separated and the aq. phase was extracted with Et₂O (10 mL). The combined organic phases were washed sequentially with 1 normal (N) aq. hydrogen chloride (HCl; 20 mL) and sat. aq. sodium bicarbonate (NaHCO₃; 20 mL), dried over MgSO₄, and filtered. The solvent was evaporated and the crude oil was purified by normal and reverse phase flash column chromatography (SiO₂, 1→40% acetone in hexanes followed by Cis, 10→100% acetonitrile (CH₃CN) in H₂O) to afford the title compound (116.4 mg, 11%) as a colorless oil: ¹H NMR (400 MHz, CDCl₃) δ 7.40-7.28 (m, 5H), 7.08-7.01 (m, 2H), 6.93 (t, J=8.7 Hz, 1H), 4.61 (d, J=11.5 Hz, 1H), 4.39 (d, J=11.5 Hz, 1H), 3.87 (td, J=5.9, 2.1 Hz, 1H), 3.49 (p, J=6.1 Hz, 1H), 3.15 (ddd, J=10.1, 5.5, 4.2 Hz, 1H), 3.01 (dd, J=13.7, 4.2 Hz, 1H), 2.86-2.80 (m, 2H), 2.79 (s, 1H), 1.81 (s, 3H), 1.28 (d, J=6.1 Hz, 3H); ¹⁹F NMR (376 MHz, CDCl₃) δ −116.64; ESIMS m/z 331 ([M+H]⁺).

### Example 4, Steps 2 and 3: Preparation of (2R,3R,4S)-4-(benzyloxy)-1-(4-fluorophenyl)pentane-2,3-diol

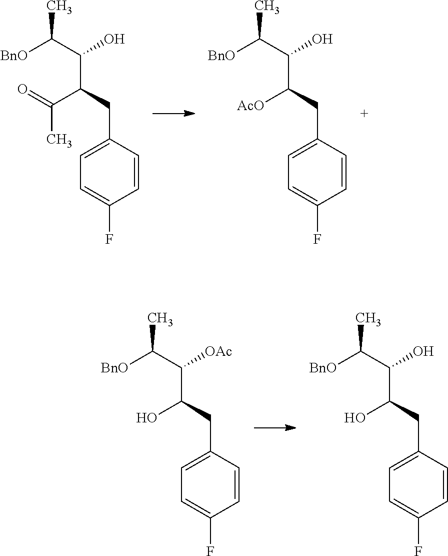 [SMILES: CC(=O)[C@H](Cc1ccc(F)cc1)[C@@H](O)[C@H](C)OCc1ccccc1 ; CC(=O)O[C@H](Cc1ccc(F)cc1)[C@@H](O)[C@H](C)OCc1ccccc1 ; CC(=O)O[C@H]([C@H](O)Cc1ccc(F)cc1)[C@H](C)OCc1ccccc1 ; C[C@H](OCc1ccccc1)[C@H](O)[C@H](O)Cc1ccc(F)cc1]

Step 2:

To a solution of (3R,4R,5S)-5-(benzyloxy)-3-(4-fluorobenzyl)-4-hydroxyhexan-2-one (3.12 g, 9.44 mmol) in DCM (94 mL) at −15° C. were added peroxybis(trimethylsilane) (4.00 mL, 18.9 mmol) and trimethylsilyl trifluoromethanesulfonate (TMSOTf; 3.42 mL, 18.9 mmol). The reaction mixture was stirred between −10 and −15° C. for 50 min, diluted with DCM, and quenched with sat. aq. NaHCO₃ followed by sat. aq. sodium sulfite (Na₂SO₃). The reaction mixture was removed from the cold bath and the phases were separated. The aq. phase was extracted with DCM (2×) and the combined organic phases were washed sequentially with 1 N HCl and sat. aq. NaHCO₃, dried over Na₂SO₄, filtered, and concentrated. The resulting oil was purified by flash column chromatography (SiO₂, 1→50% acetone in hexanes) to afford a 2:1 mixture of the two acetate regioisomers, (2R,3S,4S)-4-(benzyloxy)-1-(4-fluorophenyl)-3-hydroxypentan-2-yl acetate and (2R,3R,4S)-4-(benzyloxy)-1-(4-fluorophenyl)-2-hydroxypentan-3-yl acetate respectively, (3.01 g, 92%) as a honey colored oil.

Step 3:

To a solution of the acetate regioisomers (3.01 g, 8.69 mmol) in MeOH (70 mL) and water (17 mL) was added potassium carbonate (K₂CO₃; 6.00 g, 43.4 mmol), and the mixture was stirred for 3 h at room temperature, diluted with DCM (100 mL), and poured into 1 N HCl (150 mL). The phases were separated and the aq. phase was extracted with DCM (2×100 mL). The combined organic phases were passed through a phase separator cartridge and the solvent was evaporated. The crude waxy solid was purified by flash column chromatography (SiO₂, 1→40% acetone in hexanes) to afford the title compound (1.60 g, 61%) as a white solid: ¹H NMR (400 MHz, CDCl₃) δ 7.39-7.27 (m, 5H), 7.18 (dd, J=8.4, 5.6 Hz, 2H), 6.99 (t, J=8.7 Hz, 2H), 4.63 (d, J=11.6 Hz, 1H), 4.47 (d, J=11.6 Hz, 1H), 3.87-3.71 (m, 2H), 3.66 (dd, J=7.0, 4.8 Hz, 1H), 3.03 (dd, J=13.9, 2.9 Hz, 1H), 2.66 (dd, J=13.9, 9.1 Hz, 1H), 2.44 (brs, 1H), 2.03 (brs, 1H), 1.28 (d, J=6.2 Hz, 3H); ¹⁹F NMR (376 MHz, CDCl₃) δ −116.68; ESIMS m/z 305 ([M+H]⁺).

### Example 5, Step 1: Preparation of (2R,3S,4S)-2-(allyloxy)-4-(benzyloxy)-1-(4-fluorophenyl)pentan-3-ol

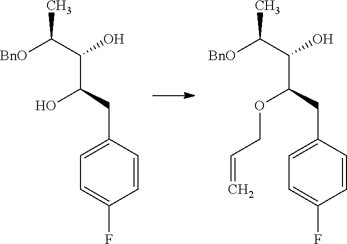 [SMILES: C[C@H](OCc1ccccc1)[C@H](O)[C@H](O)Cc1ccc(F)cc1 ; C=CCO[C@H](Cc1ccc(F)cc1)[C@@H](O)[C@H](C)OCc1ccccc1]

To a solution of (2R,3R,4S)-4-(benzyloxy)-1-(4-fluorophenyl)pentane-2,3-diol (1.81 g, 5.95 mmol), 2,2-diphenyl-1,3,2-oxazaborolidin-3-ium-2-uide (0.134 g, 0.595 mmol), potassium iodide (KI; 0.987 g, 5.95 mmol), and K₂CO₃ (0.986 g, 7.14 mmol) in CH₃CN (60 mL) was added allyl bromide (0.772 mL, 8.92 mmol), and the reaction mixture was warmed to and stirred at 60° C. overnight. The mixture was filtered through Celite® and concentrated. The crude oil was purified by flash column chromatography (SiO₂, 1→25% EtOAc in hexanes) to afford the title compound (1.30 g, 64%) as a colorless oil: ¹H NMR (500 MHz, CDCl₃) δ 7.38-7.25 (m, 5H), 7.19-7.11 (m, 2H), 6.97-6.90 (m, 2H), 5.69 (ddt, J=16.4, 10.8, 5.6 Hz, 1H), 5.11 (d, J=17.2 Hz, 1H), 5.07 (d, J=10.3 Hz, 1H), 4.60 (d, J=11.6 Hz, 1H), 4.40 (d, J=11.5 Hz, 1H), 3.84 (ddd, J=12.8, 5.7, 1.6 Hz, 1H), 3.79 (ddd, J=12.6, 5.7, 1.6 Hz, 1H), 3.71 (dq, J=11.5, 5.6 Hz, 2H), 3.62 (ddd, J=8.5, 5.8, 3.1 Hz, 1H), 2.91 (dd, J=14.2, 3.2 Hz, 1H), 2.76 (dd, J=14.2, 7.9 Hz, 1H), 2.32 (s, 1H), 1.29 (d, J=5.9 Hz, 3H); ¹³C NMR (126 MHz, CDCl₃) δ 161.49 (d, J=243.7 Hz), 138.33, 134.50, 134.45 (d, J=3.4 Hz), 131.14 (d, J=7.7 Hz), 128.43, 127.73, 127.71, 116.95, 114.81 (d, J=21.0 Hz), 79.95, 75.07, 73.41, 71.06, 70.57, 34.85, 14.74; ESIMS m/z 345 ([M+H]⁺).

### Example 5, Step 2A: Preparation of (((2R,3S,4S)-2-(allyloxy)-4-(benzyloxy)-1-(4-fluorophenyl)pentan-3-yl)oxy)triisopropylsilane

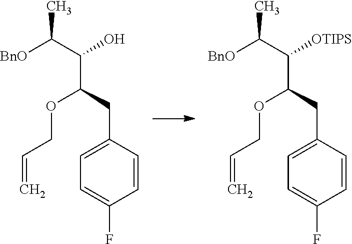

To a solution of (2R,3S,4S)-2-(allyloxy)-4-(benzyloxy)-1-(4-fluorophenyl)pentan-3-ol (1.3 g, 3.77 mmol) and 2,6-lutidine (0.55 mL, 4.72 mmol) in DCM (13 mL) at 0° C. was added triisopropylsilyl trifluoromethanesulfonate (1.27 mL, 4.72 mmol). Upon completion of the addition, the flask was removed from the cold bath and stirred at room temperature over the weekend. The reaction was quenched with sat. aq. NaHCO₃ and then the phases were separated. The aq. phase was extracted with DCM (3×) and the combined organic phases were dried by passing through a phase separator cartridge. The solvent was evaporated and the crude oil was purified by flash column chromatography (SiO₂, 0→10% acetone in hexanes) to afford the title compound (1.89 g, 100%) as a colorless oil: ¹H NMR (400 MHz, CDCl₃) δ 7.34 (d, J=4.4 Hz, 4H), 7.31-7.27 (m, 1H), 7.14-7.06 (m, 2H), 6.96-6.85 (m, 2H), 5.68-5.50 (m, 1H), 5.04 (dq, J=17.3, 1.8 Hz, 1H), 4.99 (dq, J=10.4, 1.4 Hz, 1H), 4.61 (d, J=11.7 Hz, 1H), 4.45 (d, J=11.7 Hz, 1H), 3.96 (dd, J=4.6, 2.7 Hz, 1H), 3.90 (ddt, J=12.6, 5.4, 1.5 Hz, 1H), 3.69-3.62 (m, 1H), 3.62-3.56 (m, 2H), 2.86 (dd, J=14.0, 3.6 Hz, 1H), 2.76 (dd, J=14.0, 9.3 Hz, 1H), 1.29 (d, J=6.3 Hz, 3H), 1.15-1.06 (m, 21H); ¹⁹F NMR (376 MHz, CDCl₃) δ −117.89; ESIMS m/z 523 ([M+Na]⁺).

### Example 5, Step 2B: Preparation of 1-((2R,3S,4S)-2-(allyloxy)-4-(benzyloxy)-3-(cyclopropylmethoxy)pentyl)-4-methoxybenzene

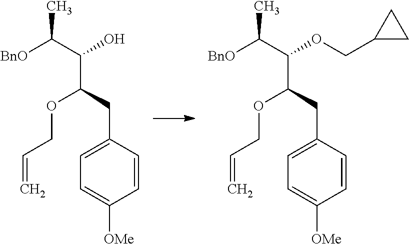 [SMILES: C=CCO[C@H](Cc1ccc(OC)cc1)[C@@H](O)[C@H](C)OCc1ccccc1 ; C=CCO[C@H](Cc1ccc(OC)cc1)[C@@H](OCC1CC1)[C@H](C)OCc1ccccc1]

To a solution of (2R,3S,4S)-2-(allyloxy)-4-(benzyloxy)-1-(4-methoxyphenyl)pentan-3-ol (340 mg, 0.954 mmol) in anhydrous THF (10 mL) was added potassium tert-butoxide (214 mg, 1.908 mmol), and the reaction mixture was stirred at room temperature for 15 min. The resulting yellow solution was treated with (bromomethyl)cyclopropane (139 μL, 1.43 mmol), and the reaction mixture was stirred at room temperature for 20 h, treated with additional potassium tert-butoxide (100 mg, 1.36 mmol) and (bromomethyl)cyclopropane (70 μL, 0.72 mmol), and warmed to and stirred at 40° C. for 4 h. The reaction was cooled, diluted with water (20 mL), and extracted with Et₂O (3×20 mL). The organic extracts were combined, dried over MgSO₄, filtered, and concentrated to provide an oil, which was purified by flash column chromatography (SiO₂) to give the title compound as well as 184 mg of recovered starting material. The recovered starting material was resubjected to the reaction conditions to provide the title compound (combined yield: 285 mg, 73%) as a colorless oil: ¹H NMR (500 MHz, CDCl₃) δ 7.37-7.26 (m, 5H), 7.20-7.10 (m, 2H), 6.85-6.76 (m, 2H), 5.69 (ddt, J=17.2, 10.4, 5.6 Hz, 1H), 5.09 (dq, J=17.2, 1.7 Hz, 1H), 5.06-5.01 (m, 1H), 4.56 (dd, J=11.8, 2.6 Hz, 1H), 4.42 (d, J=11.8 Hz, 1H), 3.83 (ddt, J=12.6, 5.6, 1.4 Hz, 1H), 3.79 (s, 3H), 3.73 (ddt, J=12.6, 5.6, 1.4 Hz, 1H), 3.69-3.61 (m, 2H), 3.51 (dd, J=6.9, 2.1 Hz, 2H), 3.42 (t, J=5.1 Hz, 1H), 2.92 (dd, J=14.2, 3.5 Hz, 1H), 2.74 (dd, J=14.2, 8.4 Hz, 1H), 1.28 (d, J=6.2 Hz, 3H), 1.16-1.04 (m, 1H), 0.57-0.48 (m, 2H), 0.26-0.20 (m, 2H); ¹³C NMR (126 MHz, CDCl₃) δ 157.87, 138.74, 135.00, 131.66, 130.57, 128.30, 127.62, 127.41, 116.42, 113.49, 82.57, 80.83, 75.38, 71.30, 70.70, 55.23, 36.09, 15.57, 11.11, 3.09, 2.92; ESIMS m/z 411 ([M+H]⁺).

### Example 6, Steps 1 and 2: Preparation of (Z)-methyl 4-((2R,3S)-2-(benzyloxy)-3-((4-methoxybenzyl)oxy)butoxy)-2-((tert-butoxycarbonyl)amino)but-2-enoate

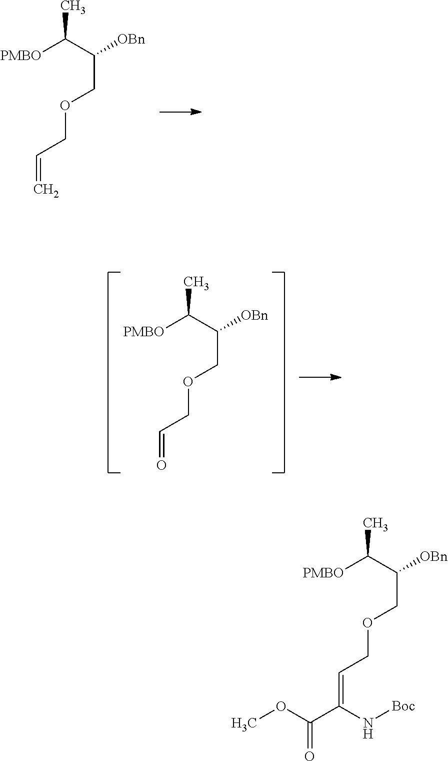

Step 1:

To a solution of 1-((((2S,3R)-4-(allyloxy)-3-(benzyloxy)butan-2-yl)oxy)methyl)-4-methoxybenzene (3.13 g, 8.78 mmol) in DCM (20 mL) and MeOH (2 mL) was added Sudan III indicator (75 μL of a 0.1% DCM solution), and the reaction mixture was cooled to −78° C. in a dry ice/acetone bath. The flask was attached to an ozone generator and 03 was bubbled through the solution until the solution became colorless (˜30 min), and then O₂ was bubbled through the solution for 5 min. While still at −78° C., the reaction mixture was treated with a solution of triphenyl phosphine (PPh₃; 3.45 g, 13.2 mmol) in DCM (5 mL), and the flask was removed from the cold bath, fitted with a balloon filled with N₂, and stirred at room temperature for 3 h. The solvent was evaporated to afford the intermediate aldehyde, 2-((2R,3S)-2-(benzyloxy)-3-((4-methoxybenzyl)oxy)butoxy)acetaldehyde, as a colorless oil.

Step 2:

To a solution of 2-((2R,3S)-2-(benzyloxy)-3-((4-methoxybenzyl)oxy)butoxy)-acetaldehyde and methyl 2-((tert-butoxycarbonyl)amino)-2-(dimethoxyphosphoryl)acetate (2.66 g, 8.96 mmol) in DCM (30 mL) was added 2,3,4,6,7,8,9,10-Octahydropyrimido[1,2-a]azepine (DBU; 1.38 mL, 9.22 mmol) a 0° C., and the reaction mixture was allowed to slowly warm to room temperature and stirred for 18 h. The solvent was evaporated and the crude residue was purified by flash column chromatography (SiO₂, 0→40% EtOAc in hexanes) to afford a predominantly single isomer of the title compound (2.4 g, 46%) as a colorless oil: ¹H NMR (400 MHz, CDCl₃) major isomer δ 7.39-7.29 (m, 4H), 7.31-7.20 (m, 3H), 6.90-6.81 (m, 2H), 6.54-6.46 (m, 2H), 4.76-4.60 (m, 2H), 4.52 (d, J=11.4 Hz, 1H), 4.44 (d, J=11.3 Hz, 1H), 4.16 (d, J=5.7 Hz, 2H), 3.79 (s, 3H), 3.79 (s, 3H), 3.73-3.51 (m, 4H), 1.45 (s, 9H), 1.23 (d, J=6.3 Hz, 3H); ¹³C NMR (101 MHz, CDCl₃) major isomer δ 164.93, 159.10, 152.96, 138.69, 130.78, 129.25, 128.75, 128.26, 127.86, 127.49, 126.08, 113.74, 80.93, 80.88, 74.43, 72.73, 70.80, 70.66, 68.26, 55.27, 52.56, 28.15, 16.09; ESIMS m/z 531 ([M+H]⁺).

### Example 6, Step 3: Preparation of (S)-methyl 4-((2R,3S)-2-(benzyloxy)-3-((4-methoxybenzyl)oxy)butoxy)-2-((tert-butoxycarbonyl)amino)butanoate

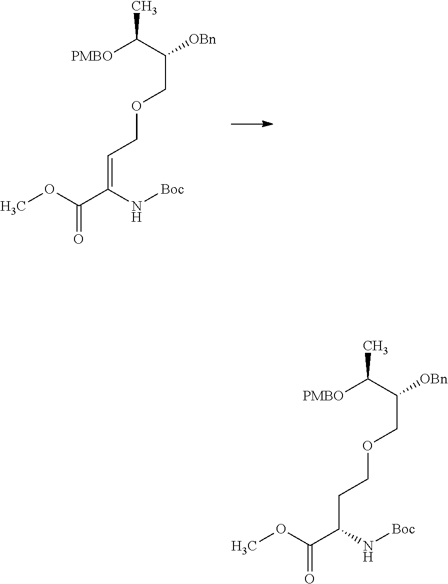

A solution of (Z)-methyl 4-((2R,3S)-2-(benzyloxy)-3-((4-methoxybenzyl)oxy)butoxy)-2-((tert-butoxycarbonyl)amino)but-2-enoate (2.4 g, 4.53 mmol) in MeOH (23 mL) was added to a 45 mL steel high pressure reactor. The solution was sparged with N₂ for 5 min, treated with (+)-1,2-bis((2S,5S)-2,5-diethylphospholano)benzene(cyclooctadiene)rhodium (I) trifluoromethane-sulfonate ((S,S)-Et-DuPhos-Rh; 0.033 g, 0.045 mmol), and the reactor was sealed, pressurized with hydrogen gas (H₂) to 200 pounds per square inch (psi), and vented. After repeating this process 3×, the reactor was pressurized to 200 psi with H₂ and the reaction mixture was stirred vigorously at room temperature for 15 h. The solvent was evaporated and the crude residue was purified by flash column chromatography (SiO₂, 0→50% EtOAc in hexanes) to afford the title compound (2.04 g, 85%) as a colorless oil: ¹H NMR (400 MHz, CDCl₃) δ 7.40-7.21 (m, 7H), 6.91-6.82 (m, 2H), 5.52 (d, J=8.2 Hz, 1H), 4.72 (d, J=11.8 Hz, 1H), 4.64 (d, J=11.8 Hz, 1H), 4.53 (d, J=11.3 Hz, 1H), 4.49-4.35 (m, 2H), 3.80 (s, 3H), 3.74-3.63 (m, 4H), 3.60-3.47 (m, 5H), 2.15-2.02 (m, 1H), 2.06-1.94 (m, 1H), 1.41 (s, 9H), 1.23 (d, J=6.3 Hz, 3H); ¹³C NMR (101 MHz, CDCl₃) δ 172.92, 159.09, 155.47, 138.74, 130.85, 129.24, 128.26, 127.86, 127.47, 113.73, 80.91, 79.72, 74.56, 72.66, 71.05, 70.82, 67.97, 55.28, 52.21, 51.96, 31.84, 28.32, 16.05; ESIMS m/z 533 ([M+H]⁺).

### Example 7, Step 1: Preparation of (S)-4-(((2R,3S,4S)-4-(benzyloxy)-1-(4-fluorophenyl)-3-((triisopropylsilyl)oxy)pentan-2-yl)oxy)-2-((tert-butoxycarbonyl)amino)butanoic acid

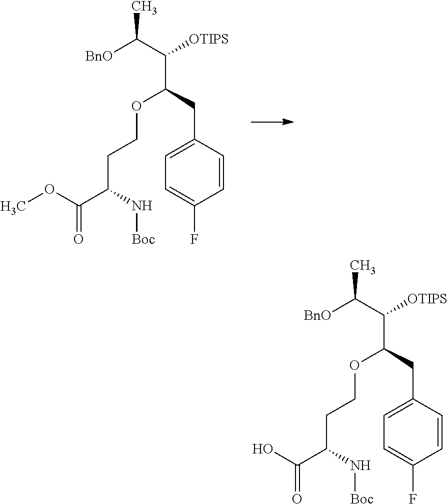

To a solution of (S)-methyl 4-(((2R,3S,4S)-4-(benzyloxy)-1-(4-fluorophenyl)-3-((triisopropylsilyl)oxy)pentan-2-yl)oxy)-2-((tert-butoxycarbonyl)amino)butanoate (1.94 g, 2.87 mmol) in THF (19 mL) and water (10 mL) was added lithium hydroxide monohydrate (LiOH—H₂O; 0.361 g, 8.61 mmol), and the reaction mixture was stirred at room temperature for 3 h. The mixture was diluted with EtOAc (50 mL) and washed sequentially with 0.2 N HCl (50 mL) and brine. The organic phase was dried over Na₂SO₄, filtered, and concentrated to afford the title compound (1.9 g, 100%) as a white foam: ¹H NMR (400 MHz, CDCl₃) δ 7.36-7.32 (m, 4H), 7.32-7.27 (m, 1H), 7.12-7.04 (m, 2H), 6.97-6.85 (m, 2H), 5.34 (d, J=7.1 Hz, 1H), 4.63 (d, J=11.6 Hz, 1H), 4.46 (d, J=11.6 Hz, 1H), 4.07 (q, J=7.1, 5.6 Hz, 1H), 3.94 (dd, J=4.9, 2.1 Hz, 1H), 3.71-3.58 (m, 3H), 3.25 (s, 1H), 2.89 (dd, J=14.3, 3.8 Hz, 1H), 2.77 (dd, J=14.3, 9.5 Hz, 1H), 1.96 (ddt, J=14.7, 9.8, 4.9 Hz, 1H), 1.90-1.81 (m, 1H), 1.45 (s, 9H), 1.30 (d, J=6.2 Hz, 3H), 1.09 (q, J=2.7, 2.1 Hz, 21H); ¹⁹F NMR (376 MHz, CDCl₃) δ −116.99; ESIMS m/z 684 ([M+Na]⁺).

### Example 7, Step 2: Preparation of (S)-2-((tert-butoxycarbonyl)amino)-4-(((2R,3S,4S)-1-(4-fluorophenyl)-4-hydroxy-3-((triisopropylsilyl)oxy)pentan-2-yl)oxy)butanoic acid

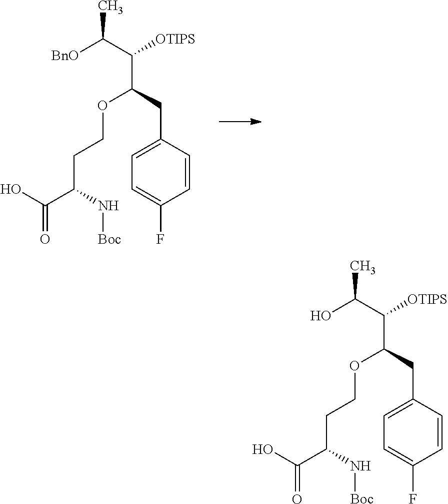

To a solution of (S)-4-(((2R,3S,4S)-4-(benzyloxy)-1-(4-fluorophenyl)-3-((triisopropyl-silyl)oxy)pentan-2-yl)oxy)-2-((tert-butoxycarbonyl)amino)butanoic acid (635 mg, 0.959 mmol) in EtOAc (9.6 mL) was added 10% palladium on carbon (Pd/C; 51.0 mg, 0.048 mmol). The mixture was placed under approximately 1 atmosphere (balloon) of H₂ and the flask was evacuated under vacuum (repeated 2×). The reaction mixture was again placed under an atmosphere of H₂ and stirred overnight at room temperature. The mixture was filtered through a pad of Celite® and concentrated to afford the title compound (540 mg, 98%) as a white foam: ¹H NMR (400 MHz, CDCl₃) δ 7.19 (dd, J=8.5, 5.5 Hz, 2H), 6.97 (t, J=8.7 Hz, 2H), 5.38-5.24 (m, 1H), 4.19 (q, J=5.8 Hz, 1H), 3.99 (dt, J=11.2, 6.2 Hz, 1H), 3.82 (dd, J=5.0, 2.0 Hz, 1H), 3.68-3.52 (m, 2H), 3.32-3.15 (m, 1H), 2.97 (dd, J=14.2, 4.3 Hz, 1H), 2.85 (dd, J=14.2, 9.3 Hz, 1H), 2.05-1.93 (m, 1H), 1.93-1.81 (m, 1H), 1.45 (s, 9H), 1.29 (d, J=6.4 Hz, 3H), 1.08 (s, 21H); ¹⁹F NMR (376 MHz, CDCl₃) δ −116.94; ESIMS m/z 594 ([M+Na]⁺).

### Example 8, Step 1: Preparation of (S)-methyl 4-((2R,3S)-2-(benzyloxy)-3-hydroxybutoxy)-2-((tert-butoxycarbonyl)amino)butanoate

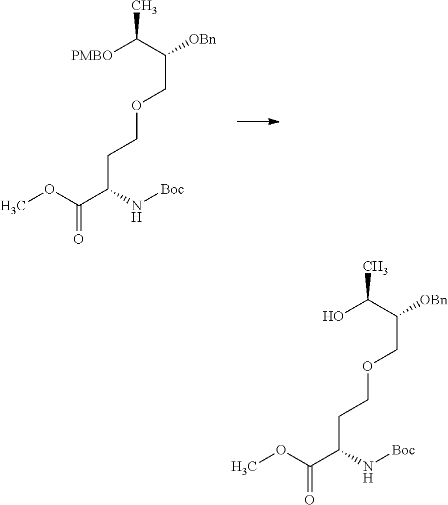

To a solution of (S)-methyl 4-((2R,3S)-2-(benzyloxy)-3-((4-methoxybenzyl)-oxy)butoxy)-2-((tert-butoxycarbonyl)amino)butanoate (2.04 g, 3.84 mmol) in DCM (14 mL) and H₂O (1.4 mL) was added 4,5-dichloro-3,6-dioxocyclohexa-1,4-diene-1,2-dicarbonitrile (DDQ; 0.915 g, 4.03 mmol) at 0° C., and the mixture was stirred vigorously at this temperature for 1 h. The reaction mixture was partitioned between 1 N sodium hydroxide (NaOH; 4.03 mL, 4.03 mmol), H₂O (20 mL), and DCM (20 mL), and the phases were separated. The aq. phase was extracted with DCM (3×25 mL) and the combined organic phases were washed with brine (10 mL), dried by passing through a phase separator cartridge, and evaporated. The crude residue was purified by flash column chromatography (SiO₂, 0→80% EtOAc in hexanes) to afford the title compound (1.22 g, 77%) as a colorless oil: ¹H NMR (400 MHz, CDCl₃) δ 7.40-7.25 (m, 5H), 5.41 (d, J=8.6 Hz, 1H), 4.71 (d, J=11.8 Hz, 1H), 4.62-4.46 (m, 2H), 4.04 (q, J=6.1 Hz, 1H), 3.73 (s, 3H), 3.64-3.52 (m, 3H), 3.45-3.28 (m, 2H), 3.17 (d, J=5.1 Hz, 1H), 2.25-2.11 (m, 1H), 1.88-1.73 (m, 1H), 1.43 (s, 9H), 1.24 (d, J=6.4 Hz, 3H); ¹³C NMR (126 MHz, CDCl₃) δ 173.21, 155.53, 138.34, 128.37, 127.93, 127.71, 82.08, 80.09, 72.31, 69.68, 67.34, 66.83, 52.35, 51.16, 32.66, 28.32, 19.22; ESIMS m/z 413 ([M+H]⁺).

### Example 8, Step 2: Preparation of (S)-4-((2R,3S)-2-(benzyloxy)-3-hydroxybutoxy)-2-((tert-butoxycarbonyl)amino)butanoic acid

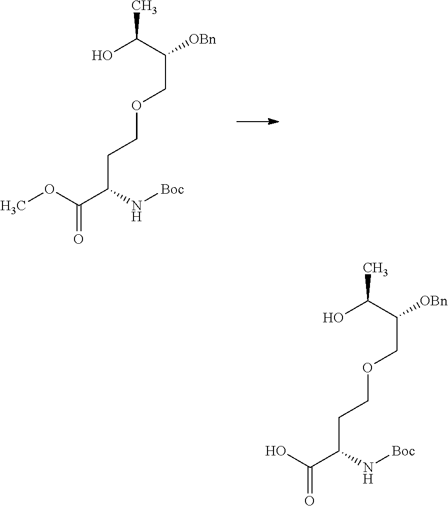 [SMILES: COC(=O)[C@H](CCOC[C@@H](OCc1ccccc1)[C@H](C)O)NC(=O)OC(C)(C)C ; C[C@H](O)[C@@H](COCC[C@H](NC(=O)OC(C)(C)C)C(=O)O)OCc1ccccc1]

To a solution of (S)-methyl 4-((2R,3S)-2-(benzyloxy)-3-hydroxybutoxy)-2-((tert-butoxycarbonyl)amino)butanoate (1.21 g, 2.94 mmol) in THF (7.8 mL) and H₂O (3.9 mL) was added LiOH—H₂O (0.370 g, 8.82 mmol), and the reaction mixture was stirred at room temperature for 4 h. The mixture was diluted with Et₂O (30 mL) and quenched with 1 N HCl (11.8 mL, 11.8 mmol). The phases were separated and the aq. phase was extracted with Et₂O (2×30 mL). The combined organic phases were washed with brine (15 mL), dried over Na₂SO₄, and filtered. The solvent was then evaporated to afford the title compound (1.17 g, 100%) as a viscous oil: ¹H NMR (500 MHz, CDCl₃) δ 7.38-7.25 (m, 5H), 5.56 (d, J=7.7 Hz, 1H), 4.69 (d, J=11.8 Hz, 1H), 4.58 (d, J=11.8 Hz, 1H), 4.46 (d, J=6.1 Hz, 1H), 4.02 (q, J=6.4 Hz, 1H), 3.67-3.56 (m, 3H), 3.49 (dt, J=9.9, 5.0 Hz, 1H), 3.39-3.33 (m, 1H), 2.18 (d, J=3.9 Hz, 1H), 1.97 (s, 1H), 1.43 (s, 9H), 1.23 (d, J=6.4 Hz, 3H); ¹³C NMR (126 MHz, CDCl₃) δ 175.20, 155.81, 138.12, 128.41, 127.97, 127.80, 81.61, 80.32, 72.30, 69.69, 67.68, 67.21, 51.40, 32.12, 28.31, 19.06; ESIMS m/z 399 ([M+H]⁺).

### Example 9: Preparation of tert-butyl ((3R,4S,7S)-3-(benzyloxy)-4-methyl-6-oxo-1,5-dioxonan-7-yl)carbamate (Compound 23)

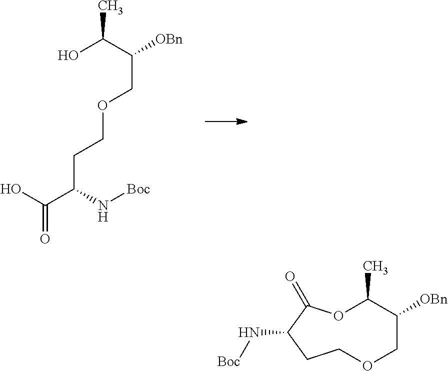 [SMILES: C[C@H](O)[C@@H](COCC[C@H](NC(=O)OC(C)(C)C)C(=O)O)OCc1ccccc1 ; C[C@@H]1OC(=O)[C@@H](NC(=O)OC(C)(C)C)CCOC[C@H]1OCc1ccccc1]

To a stirred solution of 2-methyl-6-nitrobenzoic anhydride (MNBA; 2.03 g, 5.88 mmol) and N,N-dimethylpyridin-4-amine (DMAP; 2.16 g, 17.7 mmol) in DCM (294 mL) was added a solution of (S)-4-((2R,3S)-2-(benzyloxy)-3-hydroxybutoxy)-2-((tert-butoxycarbonyl)amino)-butanoic acid (1.17 g, 2.94 mmol) in DCM (147 mL, 0.02 M) at room temperature over a 5 h period using a syringe pump. Upon completion of the addition, the reaction mixture was stirred overnight at room temperature and then the solvent was evaporated. The residue was treated with DCM (50 mL) and Celite® and the solvent was evaporated. The adsorbed material was purified using flash column chromatography (SiO₂, 0→60% EtOAc in hexanes) to afford the title compound (900 mg, 81%) as a sticky oil: ¹H NMR (400 MHz, CDCl₃) δ 7.37-7.28 (m, 5H), 5.32 (d, J=7.3 Hz, 1H), 5.23 (p, J=6.6 Hz, 1H), 4.65-4.51 (m, 2H), 4.28 (q, J=7.2 Hz, 1H), 3.86-3.78 (m, 1H), 3.73 (dd, J=11.8, 5.6 Hz, 1H), 3.58 (dd, J=11.8, 3.6 Hz, 1H), 3.52-3.38 (m, 2H), 2.40-2.24 (m, 1H), 1.78-1.68 (m, 1H), 1.44 (s, 9H), 1.35 (d, J=6.7 Hz, 3H); ¹³C NMR (101 MHz, CDCl₃) δ 172.89, 155.04, 137.75, 128.45, 127.89, 127.85, 79.82, 78.38, 73.48, 72.22, 68.57, 66.20, 51.31, 33.15, 28.33, 18.92; ESIMS m/z 381 ([M+H]⁺).

### Example 10: Preparation of tert-butyl ((2R,3S,4S,7S)-2-(4-fluorobenzyl)-3-hydroxy-4-methyl-6-oxo-1,5-dioxonan-7-yl)carbamate (Compound 4)

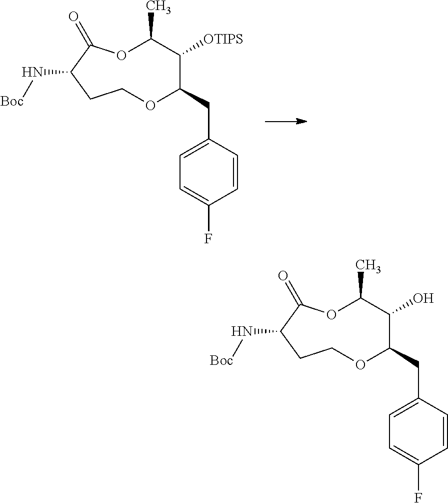

To a solution of tert-butyl ((2R,3S,4S,7S)-2-(4-fluorobenzyl)-4-methyl-6-oxo-3-((triisopropylsilyl)oxy)-1,5-dioxonan-7-yl)carbamate (1.11 g, 2.00 mmol) in THF (20 mL) was added tetrabutylammonium fluoride (TBAF; 2.4 mL, 2.40 mmol, 1 M in THF) dropwise at 0° C., and the reaction mixture was stirred at 0° C. for 30 min. The mixture was diluted with EtOAc, quenched with sat. aq. NaHCO₃, and the phases were separated. The aq. phase was extracted with EtOAc (3×), and the combined organic phases were washed with brine, dried over Na₂SO₄, filtered, and concentrated. The crude, pale-yellow oil was purified by flash column chromatography (SiO₂, 1→35% acetone in hexanes) to afford the title compound (535 mg, 67%) as a white solid: ¹H NMR (400 MHz, CDCl₃) δ 7.20 (dd, J=8.5, 5.5 Hz, 2H), 6.96 (t, J=8.7 Hz, 2H), 5.17 (d, J=8.2 Hz, 1H), 5.04 (dq, J=13.0, 6.7 Hz, 1H), 4.22 (q, J=8.3 Hz, 1H), 3.75 (ddd, J=10.5, 9.1, 3.0 Hz, 1H), 3.59 (td, J=9.2, 6.8 Hz, 1H), 3.51 (t, J=9.8 Hz, 1H), 3.47-3.42 (m, 1H), 3.14 (dd, J=15.2, 2.9 Hz, 1H), 2.84 (dd, J=15.2, 10.6 Hz, 1H), 2.53 (d, J=6.9 Hz, 1H), 2.25-2.10 (m, 1H), 1.51 (dtd, J=14.4, 10.2, 2.4 Hz, 1H), 1.42 (d, J=6.6 Hz, 3H), 1.42 (s, 9H); ¹⁹F NMR (376 MHz, CDCl₃) δ −116.91; ESIMS m/z 420 ([M+Na]⁺).

### Example 11: Preparation of tert-butyl ((3R,4S,7S)-3-hydroxy-4-methyl-6-oxo-1,5-dioxonan-7-yl)carbamate (Compound 24)

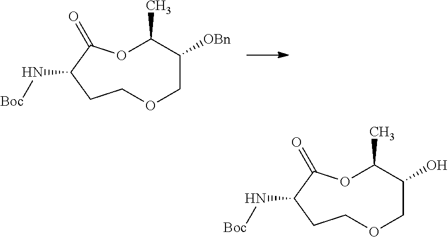 [SMILES: C[C@@H]1OC(=O)[C@@H](NC(=O)OC(C)(C)C)CCOC[C@H]1OCc1ccccc1 ; C[C@@H]1OC(=O)[C@@H](NC(=O)OC(C)(C)C)CCOC[C@H]1O]

A solution of tert-butyl ((3R,4S,7S)-3-(benzyloxy)-4-methyl-6-oxo-1,5-dioxonan-7-yl)carbamate (715 mg, 1.88 mmol) in THF (9 mL) was treated with 10% Pd/C (60.2 mg, 0.057 mmol). The reaction mixture was placed under approximately 1 atmosphere (balloon) of H₂ and the flask was evacuated under vacuum (repeated 2×). The reaction mixture was again placed under an atmosphere of H₂ and the heterogeneous mixture was stirred for 5 h at room temperature. The mixture was filtered through a pad of Celite® and concentrated to afford the title compound (518 mg, 95%) as a white solid: ¹H NMR (400 MHz, CDCl₃) δ 5.39-5.29 (m, 1H), 5.25 (d, J=7.7 Hz, 1H), 4.35-4.24 (m, 1H), 4.07 (ddd, J=10.9, 5.9, 2.8 Hz, 1H), 3.95 (ddd, J=11.2, 4.3, 1.1 Hz, 1H), 3.50-3.39 (m, 3H), 3.12 (d, J=11.1 Hz, 1H), 2.33-2.21 (m, 1H), 1.80-1.66 (m, 1H), 1.44 (s, 9H), 1.32 (d, J=6.9 Hz, 3H); ¹³C NMR (101 MHz, CDCl₃) δ 174.77, 155.08, 80.00, 76.97, 73.57, 70.29, 68.57, 51.42, 33.55, 28.30, 18.47; ESIMS m/z 312 ([M+Na]⁺).

### Example 12A: Preparation of (2R,3S,4S,7S)-7-((tert-butoxycarbonyl)amino)-2-(4-fluorobenzyl)-4-methyl-6-oxo-1,5-dioxonan-3-yl isobutyrate (Compound 8)

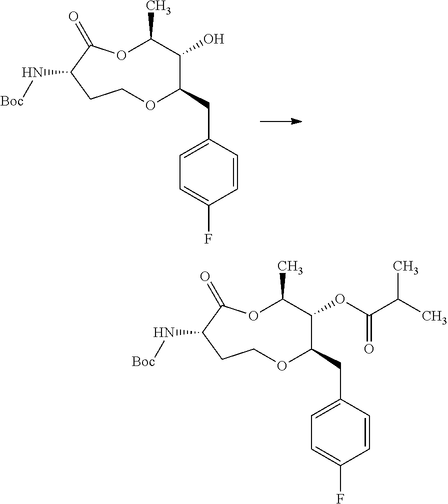

To a solution of tert-butyl ((2R,3S,4S,7S)-2-(4-fluorobenzyl)-3-hydroxy-4-methyl-6-oxo-1,5-dioxonan-7-yl)carbamate (150 mg, 0.377 mmol) and DMAP (9.22 mg, 0.075 mmol) in DCM (3.8 mL) was added triethylamine (NEt₃; 210 μL, 1.51 mmol) and isobutyryl chloride (59.3 μL, 0.566 mmol) at 0° C., and the reaction was slowly warmed to room temperature and stirred overnight. Additional isobutryl chloride (40 μL, 0.38 mmol) and NEt₃ (105 μL, 0.76 mmol) were added at room temperature and the reaction was stirred overnight. The reaction was diluted with DCM and quenched with sat. aq. NH₄Cl. The phases were separated and the aq. phase was extracted with DCM (3×). The combined organic phases were dried by passing through a phase separator cartridge and then the solvent was evaporated. The crude residue was purified by flash column chromatography (SiO₂, 1→25% acetone in hexanes) to afford the title compound (159.9 mg, 91%) as a white foam: ¹H NMR (400 MHz, CDCl₃) δ 7.15 (dd, J=8.6, 5.4 Hz, 2H), 6.97 (t, J=8.7 Hz, 2H), 5.27-5.17 (m, 1H), 5.14 (t, J=9.4 Hz, 1H), 5.08 (d, J=8.2 Hz, 1H), 4.27 (q, J=8.4 Hz, 1H), 3.95 (ddd, J=10.6, 9.3, 3.3 Hz, 1H), 3.62 (t, J=10.1 Hz, 1H), 3.50-3.42 (m, 1H), 2.87 (dd, J=15.4, 10.7 Hz, 1H), 2.60 (dd, J=15.2, 3.1 Hz, 1H), 2.50 (hept, J=7.0 Hz, 1H), 2.22 (dt, J=14.9, 7.0 Hz, 1H), 1.59-1.47 (m, 1H), 1.43 (s, 9H), 1.27 (d, J=6.3 Hz, 3H), 1.18 (d, J=7.0 Hz, 3H), 1.17 (d, J=7.0 Hz, 3H); ¹⁹F NMR (376 MHz, CDCl₃) δ −116.62; ESIMS m/z 490 ([M+Na]⁺).

### Example 12B: Preparation of tert-butyl ((2R,3S,4S,7S)-2-(4-fluorobenzyl)-4-methyl-6-oxo-3-phenoxy-1,5-dioxonan-7-yl)carbamate (Compound 5)

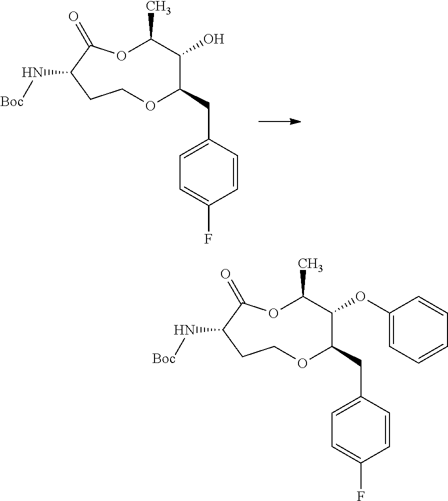

To a solution of tert-butyl ((2R,3S,4S,7S)-2-(4-fluorobenzyl)-3-hydroxy-4-methyl-6-oxo-1,5-dioxonan-7-yl)carbamate (150 mg, 0.377 mmol) in toluene (2.5 mL) were added diacetoxycopper (6.86 mg, 0.038 mmol), Bi(OAc)₂Ph₃ (316 mg, 0.566 mmol), and N, N-dicyclohexyl methylamine (96 μL, 0.453 mmol). The reaction vessel was evacuated under vacuum and backfilled with N₂ (repeated 4×), sealed, and the reaction mixture was stirred at 40° C. over the weekend. The mixture was filtered through Celite® (rinsing with EtOAc) and concentrated, and the resulting oil was purified by flash column chromatography (SiO₂, 1→25% acetone in hexanes) to afford the title compound (83.9 mg, 47%) as a white solid: ¹H NMR (400 MHz, CDCl₃) δ 7.33-7.27 (m, 2H), 7.15 (dd, J=8.5, 5.5 Hz, 2H), 7.02-6.90 (m, 5H), 5.33-5.19 (m, 1H), 5.17 (d, J=8.1 Hz, 1H), 4.44 (t, J=9.2 Hz, 1H), 4.29 (q, J=8.1 Hz, 1H), 4.01 (td, J=10.3, 9.1, 2.8 Hz, 1H), 3.58 (t, J=9.8 Hz, 1H), 3.50-3.38 (m, 1H), 2.96-2.87 (m, 1H), 2.78 (dd, J=15.1, 11.1 Hz, 1H), 2.25-2.18 (m, 1H), 1.86-1.71 (m, 1H), 1.43 (s, 9H), 1.36 (d, J=6.5 Hz, 3H); ¹⁹F NMR (376 MHz, CDCl₃) δ −116.86; ESIMS m/z 496 ([M+Na]⁺).

### Example 12C, Step 1: Preparation of tert-butyl ((2R,3S,4S,7S)-3-(allyloxy)-2-(4-fluorobenzyl)-4-methyl-6-oxo-1,5-dioxonan-7-yl)carbamate (Compound 9)

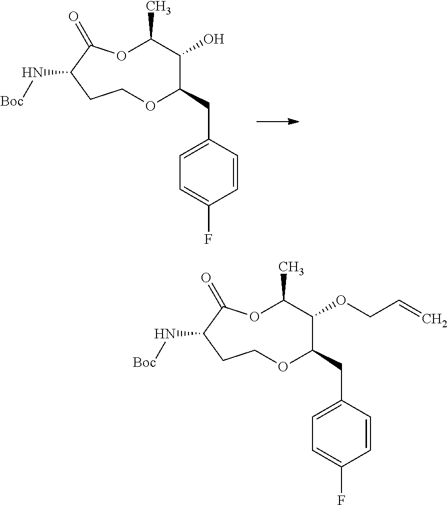

A magnetically stirred mixture of tert-butyl ((2R,3S,4S,7S)-2-(4-fluorobenzyl)-3-hydroxy-4-methyl-6-oxo-1,5-dioxonan-7-yl)carbamate (300 mg, 0.755 mmol), Pd₂(dba)₃ (34.6 mg, 0.038 mmol), and dppf (41.8 mg, 0.075 mmol), in THF (7.5 mL) was heated to 60° C. and treated dropwise with a solution of allyl tert-butyl carbonate (239 μL, 1.51 mmol) in THF (1 mL) over a 9 min period. The reaction mixture was stirred for an additional 20 min at 60° C., cooled to room temperature, and filtered through Celite® rinsing with EtOAc. The filtrate was concentrated and purified by flash column chromatography (SiO₂, 1→20% acetone in hexanes) to afford the title compound (223.5 mg, 68%) as a white foam: ¹H NMR (400 MHz, CDCl₃) δ 7.20 (dd, J=8.6, 5.5 Hz, 2H), 6.97 (t, J=8.7 Hz, 2H), 5.89 (ddt, J=17.2, 10.4, 5.6 Hz, 1H), 5.30 (dq, J=17.2, 1.6 Hz, 1H), 5.21 (dq, J=10.3, 1.3 Hz, 1H), 5.10 (d, J=8.2 Hz, 1H), 5.07-5.01 (m, 1H), 4.28-4.18 (m, 2H), 4.08 (ddt, J=12.2, 5.7, 1.4 Hz, 1H), 3.85-3.74 (m, 1H), 3.48 (t, J=9.8 Hz, 1H), 3.36 (q, J=9.2, 8.6 Hz, 2H), 3.05 (d, J=14.8 Hz, 1H), 2.79 (dd, J=14.9, 11.2 Hz, 1H), 2.21-2.10 (m, 1H), 1.57-1.48 (m, 1H), 1.47 (d, J=6.7 Hz, 3H), 1.42 (s, 9H); ¹⁹F NMR (376 MHz, CDCl₃) δ −117.01; ESIMS m/z 460 ([M+Na]⁺).

### Example 12C, Step 2: Preparation of tert-butyl ((2R,3S,4S,7S)-2-(4-fluorobenzyl)-4-methyl-6-oxo-3-propoxy-1,5-dioxonan-7-yl)carbamate (Compound 10)

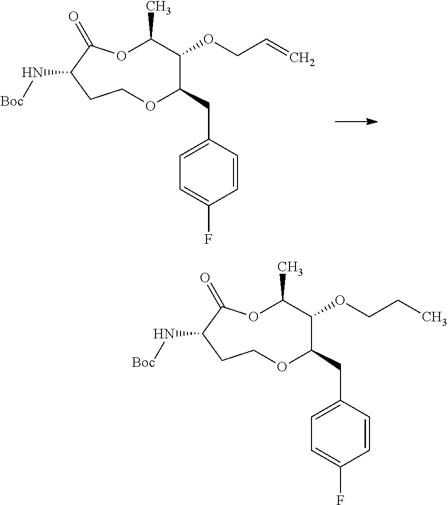

To a solution of tert-butyl ((2R,3S,4S,7S)-3-(allyloxy)-2-(4-fluorobenzyl)-4-methyl-6-oxo-1,5-dioxonan-7-yl)carbamate (110 mg, 0.251 mmol) in EtOAc (2.5 mL) was added 10% Pd/C (13.4 mg, 0.013 mmol). The mixture was placed under approximately 1 atmosphere (balloon) of H₂ and the flask was evacuated under vacuum (repeated 2×). The reaction mixture was again placed under an atmosphere of H₂ and stirred overnight at room temperature. The mixture was filtered through a pad of Celite® and concentrated to afford the title compound (110 mg, 100%) as a white foam: ¹H NMR (400 MHz, CDCl₃) δ 7.20 (dd, J=8.5, 5.5 Hz, 2H), 6.97 (t, J=8.7 Hz, 2H), 5.13 (d, J=8.2 Hz, 1H), 5.07-4.97 (m, 1H), 4.22 (q, J=8.3 Hz, 1H), 3.76 (ddd, J=11.6, 9.2, 2.6 Hz, 1H), 3.66 (dt, J=8.6, 6.6 Hz, 1H), 3.57-3.44 (m, 2H), 3.43-3.34 (m, 1H), 3.26 (t, J=9.2 Hz, 1H), 3.02 (dd, J=14.9, 2.5 Hz, 1H), 2.80 (dd, J=14.9, 11.2 Hz, 1H), 2.23-2.09 (m, 1H), 1.66-1.52 (m, 2H), 1.54-1.47 (m, 1H), 1.46 (d, J=7.1 Hz, 3H), 1.42 (s, 9H), 0.94 (t, J=7.4 Hz, 3H); ¹⁹F NMR (376 MHz, CDCl₃) δ −117.06; ESIMS m/z 440 ([M+H]⁺).

### Example 13A, Steps 1 and 2: Preparation of N-((2R,3S,4S,7S)-2-(4-fluorobenzyl)-4-methyl-6-oxo-3-propoxy-1,5-dioxonan-7-yl)-3-hydroxy-4-methoxypicolinamide (Compounds 36 and 56)

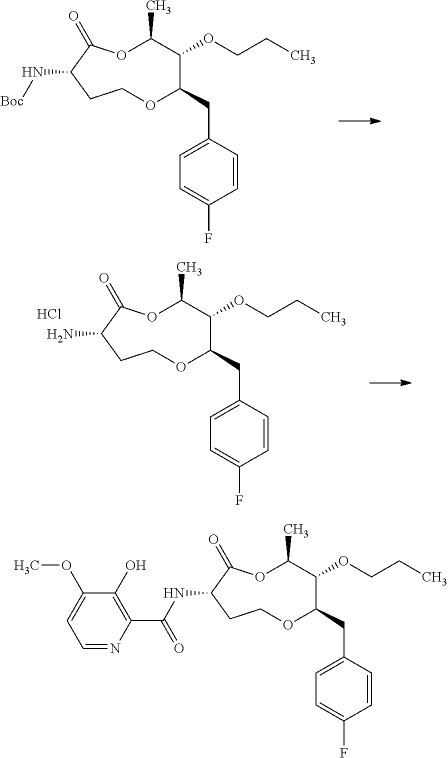

Step 1:

To a solution of tert-butyl ((2R,3S,4S,7S)-2-(4-fluorobenzyl)-4-methyl-6-oxo-3-propoxy-1,5-dioxonan-7-yl)carbamate (111 mg, 0.253 mmol) in DCM (1.7 mL) was added a 4 N solution of HCl in dioxane (1.26 mL, 5.05 mmol). After 2.5 h at room temperature, the solvent was evaporated under a stream of N₂ to provide the intermediate amine hydrochloride, (2R,3S,4S,7S)-2-(4-fluorobenzyl)-4-methyl-6-oxo-3-propoxy-1,5-dioxonan-7-aminium chloride, as a pale yellow solid: ESIMS m/z 340 ([M+H]⁺).

Step 2:

To a solution of (2R,3S,4S,7S)-2-(4-fluorobenzyl)-4-methyl-6-oxo-3-propoxy-1,5-dioxonan-7-aminium chloride and 3-hydroxy-4-methoxypicolinic acid (47.0 mg, 0.278 mmol) in DCM (1.7 mL) were added N-ethyl-N-isopropylpropan-2-amine (145 μL, 0.833 mmol) and benzotriazol-1-yloxy)tripyrrolidinophosphonium hexafluorophosphate (PyBOP; 145 mg, 0.278 mmol), and the reaction mixture was stirred under N₂ at room temperature for 4 h. The solvent was evaporated and the crude oil was purified by flash column chromatography (SiO₂, 1→50% acetone in hexanes) to afford the title compound (117 mg, 94%) as a white solid: ¹H NMR (400 MHz, CDCl₃) δ 12.06 (s, 1H), 8.53 (d, J=8.0 Hz, 1H), 7.97 (d, J=5.2 Hz, 1H), 7.22 (dd, J=8.5, 5.5 Hz, 2H), 6.98 (t, J=8.7 Hz, 2H), 6.85 (d, J=5.2 Hz, 1H), 5.12-4.99 (m, 1H), 4.62 (td, J=8.5, 7.1 Hz, 1H), 3.93 (s, 3H), 3.81 (ddd, J=11.5, 9.2, 2.6 Hz, 1H), 3.67 (dt, J=8.6, 6.6 Hz, 1H), 3.60-3.48 (m, 2H), 3.48-3.37 (m, 1H), 3.31 (t, J=9.2 Hz, 1H), 3.11-3.00 (m, 1H), 2.82 (dd, J=14.9, 11.2 Hz, 1H), 2.35-2.24 (m, 1H), 1.76-1.62 (m, 1H), 1.60 (h, J=7.1 Hz, 2H), 1.50 (d, J=6.5 Hz, 3H), 0.95 (t, J=7.4 Hz, 3H); ¹⁹F NMR (376 MHz, CDCl₃) δ −117.01; HRMS-ESI (m/z) [M+H]⁺ calcd for C₂₅H₃₂FN₂O₇, 491.2188; found, 491.2183.

### Example 13B, Steps 1 and 2: Preparation of N-((2R,3S,4S,7S)-3-(allyloxy)-2-(4-fluorobenzyl)-4-methyl-6-oxo-1,5-dioxonan-7-yl)-3-hydroxy-4-methoxypicolinamide (Compounds 35 and 55)

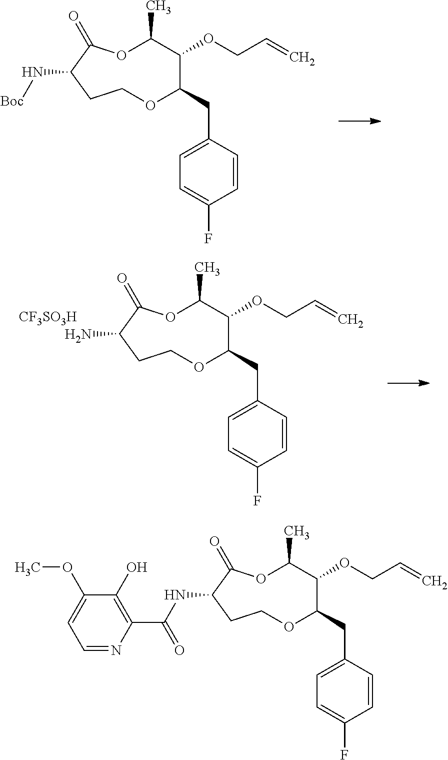

Step 1:

To a solution of tert-butyl ((2R,3S,4S,7S)-3-(allyloxy)-2-(4-fluorobenzyl)-4-methyl-6-oxo-1,5-dioxonan-7-yl)carbamate (111 mg, 0.254 mmol) in DCM (2.5 mL) were added 2,6-lutidine (89 μL, 0.76 mmol) followed by trimethylsilyl trifluoromethanesulfonate (92 μL, 0.51 mmol) dropwise at room temperature, and the reaction mixture was stirred under N₂ at room temperature for 1.5 h. The reaction mixture was treated with MeOH (0.9 mL) and was stirred overnight. The solvent was evaporated to afford the intermediate ammonium trifluoromethanesulfonate, (2R,3S,4S,7S)-3-(allyloxy)-2-(4-fluorobenzyl)-4-methyl-6-oxo-1,5-dioxonan-7-aminium trifluoromethanesulfonate: ESIMS m/z 338 ([M+H]⁺).

Step 2:

To a solution of (2R,3S,4S,7S)-3-(allyloxy)-2-(4-fluorobenzyl)-4-methyl-6-oxo-1,5-dioxonan-7-aminium trifluoromethanesulfonate and 3-hydroxy-4-methoxypicolinic acid (47.2 mg, 0.279 mmol) in DCM (2.5 mL) were added N-ethyl-N-isopropylpropan-2-amine (146 μL, 0.837 mmol) and PyBOP (145 mg, 0.279 mmol), and the reaction mixture was stirred under N₂ at room temperature overnight. The solvent was evaporated and the resulting oil was purified by flash column chromatography (SiO₂, 1→40% acetone in hexanes) to afford the title compound (107 mg, 86%) as a white foam: ¹H NMR (400 MHz, CDCl₃) δ 12.06 (s, 1H), 8.52 (d, J=7.9 Hz, 1H), 7.96 (d, J=5.2 Hz, 1H), 7.22 (dd, J=8.5, 5.5 Hz, 2H), 6.97 (t, J=8.7 Hz, 2H), 6.85 (d, J=5.3 Hz, 1H), 5.90 (ddt, J=17.2, 10.4, 5.6 Hz, 1H), 5.31 (dq, J=17.2, 1.6 Hz, 1H), 5.21 (dq, J=10.4, 1.3 Hz, 1H), 5.14-5.01 (m, 1H), 4.62 (td, J=8.5, 7.0 Hz, 1H), 4.24 (ddt, J=12.2, 5.5, 1.4 Hz, 1H), 4.10 (ddt, J=12.2, 5.7, 1.4 Hz, 1H), 3.92 (s, 3H), 3.84 (ddd, J=11.6, 9.2, 2.6 Hz, 1H), 3.55 (t, J=9.6 Hz, 1H), 3.49-3.41 (m, 1H), 3.40 (t, J=9.6 Hz, 1H), 3.08 (dt, J=14.7, 2.0 Hz, 1H), 2.88-2.79 (m, 1H), 2.38-2.22 (m, 1H), 1.79-1.62 (m, 1H), 1.51 (d, J=6.6 Hz, 3H); ¹⁹F NMR (376 MHz, CDCl₃) δ −116.94; HRMS-ESI (m/z) [M+H]⁺ calcd for C₂₅H₃₀FN₂O₇, 489.2032; found, 489.2032.

### Example 14: Preparation of 2-(((2R,3S,4S,7S)-2-(4-fluorobenzyl)-4-methyl-6-oxo-3-propoxy-1,5-dioxonan-7-yl)carbamoyl)-4-methoxypyridin-3-yl acetate (Compound 81)

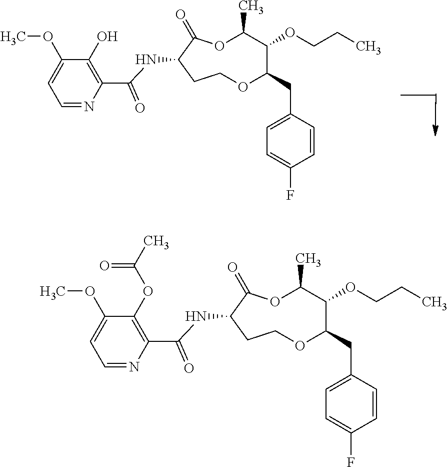

To a solution of N-((2R,3S,4S,7S)-2-(4-fluorobenzyl)-4-methyl-6-oxo-3-propoxy-1,5-dioxonan-7-yl)-3-hydroxy-4-methoxypicolinamide (38 mg, 0.077 mmol), NEt₃ (21.5 μL, 0.155 mmol), and DMAP (1.9 mg, 0.015 mmol) in DCM (0.8 mL) was added acetyl chloride (8.3 μL, 0.116 mmol), and the reaction mixture was stirred at room temperature for 3 h. The mixture was diluted with DCM, poured into sat. aq. NH₄Cl, and the phases were separated. The aq. phase was extracted with DCM (3×), and the combined organic phases were dried by passing through a phase separator cartridge and concentrated. The resulting crude oil was purified by flash column chromatography (SiO₂, 1→50% acetone in hexanes) to afford the title compound (40.9 mg, 100%) as a white foam: ¹H NMR (400 MHz, CDCl₃) δ 8.57 (d, J=8.1 Hz, 1H), 8.31 (d, J=5.4 Hz, 1H), 7.21 (dd, J=8.6, 5.5 Hz, 2H), 7.03-6.90 (m, 3H), 5.19-4.95 (m, 1H), 4.62 (td, J=8.7, 7.1 Hz, 1H), 3.89 (s, 3H), 3.79 (ddd, J=11.5, 9.2, 2.6 Hz, 1H), 3.66 (dt, J=8.6, 6.6 Hz, 1H), 3.56-3.47 (m, 2H), 3.46-3.39 (m, 1H), 3.29 (t, J=9.2 Hz, 1H), 3.09-3.00 (m, 1H), 2.81 (dd, J=14.9, 11.2 Hz, 1H), 2.38 (s, 3H), 2.34-2.22 (m, 1H), 1.71-1.52 (m, 3H), 1.47 (d, J=6.5 Hz, 3H), 0.94 (t, J=7.4 Hz, 3H); ¹⁹F NMR (376 MHz, CDCl₃) δ −117.10; HRMS-ESI (m/z) [M+H]⁺ calcd for C₂₇H₃₄FN₂O₈, 533.2294; found, 533.2295.

### Example 15: Preparation of ((2-(((2R,3S,4S,7S)-2-(4-fluorobenzyl)-4-methyl-6-oxo-3-propoxy-1,5-dioxonan-7-yl)carbamoyl)-4-methoxypyridin-3-yl)oxy)methyl acetate (Compound 79)

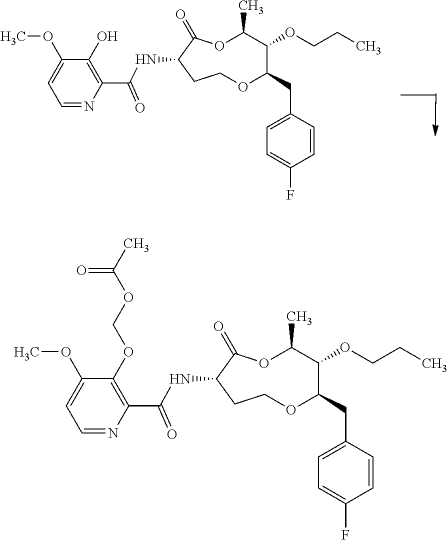

To a solution of N-((2R,3S,4S,7S)-2-(4-fluorobenzyl)-4-methyl-6-oxo-3-propoxy-1,5-dioxonan-7-yl)-3-hydroxy-4-methoxypicolinamide (60 mg, 0.122 mmol) and potassium carbonate (K₂CO₃; 33.8 mg, 0.245 mmol) in acetone (1.2 mL) was added bromomethyl acetate (16.8 μL, 0.171 mmol) dropwise. The reaction vessel was sealed and the mixture was warmed to and stirred at 55° C. for 3 h. The mixture was filtered through a fritted filter rinsing with a 3:1 mixture of hexanes and acetone. The solvent was evaporated and the crude residue was purified by flash column chromatography (SiO₂, 1→50% acetone in hexanes) to afford the title compound (47.7 mg, 69%) as a white foam: ¹H NMR (400 MHz, CDCl₃) δ 8.36 (d, J=7.9 Hz, 1H), 8.26 (d, J=5.3 Hz, 1H), 7.22 (dd, J=8.5, 5.5 Hz, 2H), 6.97 (t, J=8.7 Hz, 2H), 6.94 (d, J=5.5 Hz, 1H), 5.72 (s, 2H), 5.13-4.95 (m, 1H), 4.71-4.56 (m, 1H), 3.90 (s, 3H), 3.80 (ddd, J=11.5, 9.2, 2.6 Hz, 1H), 3.67 (dt, J=8.6, 6.5 Hz, 1H), 3.60-3.47 (m, 2H), 3.47-3.40 (m, 1H), 3.30 (t, J=9.2 Hz, 1H), 3.05 (dt, J=14.8, 2.0 Hz, 1H), 2.83 (dd, J=15.0, 11.2 Hz, 1H), 2.38-2.24 (m, 1H), 2.05 (s, 3H), 1.70-1.63 (m, 1H), 1.60 (h, J=7.1 Hz, 2H), 1.48 (d, J=6.5 Hz, 3H), 0.95 (t, J=7.4 Hz, 3H); ¹⁹F NMR (376 MHz, CDCl₃) δ −117.11; HRMS-ESI (m/z) [M+H]⁺ calcd for C₂₈H₃₆FN₂O₉, 563.2399; found, 563.2404.

### Example A: Evaluation of Fungicidal Activity: Leaf Blotch of Wheat (Mycosphaerella graminicola; Anamorph: Zymoseptoria tritici; Bayer Code SEPTTR)

Technical grades of materials were dissolved in acetone, which were then mixed with nine volumes of water containing 110 ppm Triton X-100. The fungicide solutions were applied onto wheat seedlings using an automated booth sprayer to run-off. All sprayed plants were allowed to air dry prior to further handling. All fungicides were evaluated using the aforementioned method for their activity vs. all target diseases, unless stated otherwise. Wheat leaf blotch and brown rust activity were also evaluated using track spray applications, in which case the fungicides were formulated as EC formulations, containing 0.1% Trycol 5941 in the spray solutions.

Wheat plants (variety Yuma) were grown from seed in a greenhouse in 50% mineral soil/50% soil-less Metro mix until the first leaf was fully emerged, with 7-10 seedlings per pot. These plants were inoculated with an aqueous spore suspension of Zymoseptoria tritici either prior to or after fungicide treatments. After inoculation the plants were kept in 100% relative humidity (one day in a dark dew chamber followed by two to three days in a lighted dew chamber at 20° C.) to permit spores to germinate and infect the leaf. The plants were then transferred to a greenhouse set at 20° C. for disease to develop. When disease symptoms were fully expressed on the 1^{st} leaves of untreated plants, infection levels were assessed on a scale of 0 to 100 percent disease severity. Percent disease control was calculated using the ratio of disease severity on treated plants relative to untreated plants.

### Example B: Evaluation of Fungicidal Activity: Wheat Brown Rust (Puccinia triticina; Synonym: Puccinia recondita f. sp. tritici; Bayer Code PUCCRT)

Wheat plants (variety Yuma) were grown from seed in a greenhouse in 50% mineral soil/50% soil-less Metro mix until the first leaf was fully emerged, with 7-10 seedlings per pot. These plants were inoculated with an aqueous spore suspension of Puccinia triticina either prior to or after fungicide treatments. After inoculation the plants were kept in a dark dew room at 22° C. with 100% relative humidity overnight to permit spores to germinate and infect the leaf. The plants were then transferred to a greenhouse set at 24° C. for disease to develop. Fungicide formulation, application and disease assessment followed the procedures as described in the Example A.

### Example C: Evaluation of Fungicidal Activity: Wheat Glume Blotch (Leptosphaeria nodorum; Bayer code LEPTNO)

Wheat plants (variety Yuma) were grown from seed in a greenhouse in 50% mineral soil/50% soil-less Metro mix until the first leaf was fully emerged, with 7-10 seedlings per pot. These plants were inoculated with an aqueous spore suspension of Leptosphaeria nodorum 24 hr after fungicide treatments. After inoculation the plants were kept in 100% relative humidity (one day in a dark dew chamber followed by two days in a lighted dew chamber at 20° C.) to permit spores to germinate and infect the leaf. The plants were then transferred to a greenhouse set at 20° C. for disease to develop. Fungicide formulation, application and disease assessment followed the procedures as described in the Example A.

### Example D: Evaluation of Fungicidal Activity: Apple Scab (Venturia inaequalis; Bayer Code VENTIN)

Apple seedlings (variety McIntosh) were grown in soil-less Metro mix, with one plant per pot. Seedlings with two expanding young leaves at the top (older leaves at bottom of the plants were trimmed) were used in the test. Plants were inoculated with a spore suspension of Venturia inaequalis 24 hr after fungicide treatment and kept in a 22° C. dew chamber with 100% relative humidity for 48 hr, and then moved to a greenhouse set at 20° C. for disease to develop. Fungicide formulation, application and disease assessment on the sprayed leaves followed the procedures as described in the Example A.

### Example E: Evaluation of Fungicidal Activity: Grape Powdery Mildew (Uncinula necator; Bayer Code UNCINE)

Grape seedlings (variety Carignane) were grown in soil-less Metro mix, with one plant per pot, and used in the test when approximately one month old. Plants were inoculated 24 hr after fungicide treatment by shaking spores from infected leaves over test plants. Plants were maintained in a greenhouse set at 20° C. until disease was fully developed. Fungicide formulation, application and disease assessment on the sprayed leaves followed the procedures as described in the Example A.

### Example F: Evaluation of Fungicidal Activity: Powdery Mildew of Cucumber (Erysiphe cichoracearum; Bayer Code ERYSCI)

Cucumber seedlings (variety Bush Pickle) were grown in soil-less Metro mix, with one plant per pot, and used in the test when 12 to 14 days old. Plants were inoculated with a spore suspension 24 hr following fungicide treatments. After inoculation the plants remained in the greenhouse set at 20° C. until disease was fully expressed. Fungicide formulation, application and disease assessment on the sprayed leaves followed the procedures as described in the Example A.

### Example G: Evaluation of Fungicidal Activity: Leaf Spot of Sugar Beets (Cercospora beticola; Bayer Code CERCBE)

Sugar beet plants (variety HH88) were grown in soil-less Metro mix and trimmed regularly to maintain a uniform plant size prior to test. Plants were inoculated with a spore suspension 24 hr after fungicide treatments. Inoculated plants were kept in a dew chamber at 22° C. for 48 hr then incubated in a greenhouse set at 24° C. under a clear plastic hood with bottom ventilation until disease symptoms were fully expressed. Fungicide formulation, application and disease assessment on the sprayed leaves followed the procedures as described in the Example A.

### Example H: Evaluation of Fungicidal Activity: Asian Soybean Rust (Phakopsora pachyrhizi; Bayer Code PHAKPA)

Technical grades of materials were dissolved in acetone, which were then mixed with nine volumes of water containing 0.011% Tween 20. The fungicide solutions were applied onto soybean seedlings using an automated booth sprayer to run-off. All sprayed plants were allowed to air dry prior to further handling.

Soybean plants (variety Williams 82) were grown in soil-less Metro mix, with one plant per pot. Two weeks old seedlings were used for testing. Plants were inoculated either 3 days prior to or 1 day after fungicide treatments. Plants were incubated for 24 h in a dark dew room at 22° C. and 100% relative humidity then transferred to a growth room at 23° C. for disease to develop. Disease severity was assessed on the sprayed leaves.

### Example I: Evaluation of Fungicidal Activity: Wheat Powdery Mildew (Blumeria graminis f. sp. tritici; Synonym: Erysiphe graminis f. sp. tritici; Bayer Code ERYSGT)

Wheat plants (variety Yuma) were grown from seed in a greenhouse in 50% mineral soil/50% soil-less Metro mix until the first leaf was fully emerged, with 7-10 seedlings per pot. These plants were inoculated by dusting with infected stock plants 24 hr after fungicide treatments. After inoculation the plants were kept in a greenhouse set at 20° C. for disease to develop. Fungicide formulation, application and disease assessment on the sprayed leaves followed the procedures as described in the Example A.

### Example J: Evaluation of Fungicidal Activity: Barley Powdery Mildew (Blumeria graminis f. sp. hordei; Synonym: Erysiphe graminis f. sp. hordei; Bayer Code ERYSGH)

Barley seedlings (variety Harrington) were propagated in soil-less Metro mix, with each pot having 8 to 12 plants, and used in the test when first leaf was fully emerged. Test plants were inoculated by dusting with infected stock plants 24 hr after fungicide treatments. After inoculation the plants were kept in a greenhouse set at 20° C. for disease to develop. Fungicide formulation, application and disease assessment on the sprayed leaves followed the procedures as described in the Example A.

### Example K: Evaluation of Fungicidal Activity: Barley Scald (Rhyncosporium secalis; Bayer Code RHYNSE)

Barley seedlings (variety Harrington) were propagated in soil-less Metro mix, with each pot having 8 to 12 plants, and used in the test when first leaf was fully emerged. Test plants were inoculated by an aqueous spore suspension of Rhyncosporium secalis 24 hr after fungicide treatments. After inoculation the plants were kept in a dew room at 20° C. with 100% relative humidity for 48 hr. The plants were then transferred to a greenhouse set at 20° C. for disease to develop. Fungicide formulation, application and disease assessment on the sprayed leaves followed the procedures as described in the Example A.

### Example L: Evaluation of Fungicidal Activity: Rice Blast (Magnaporthe grisea; Anamorph: Pyricularia oryzae; Bayer Code PYRIOR)

Rice seedlings (variety Japonica) were propagated in soil-less Metro mix, with each pot having 8 to 14 plants, and used in the test when 12 to 14 days old. Test plants were inoculated with an aqueous spore suspension of Pyricularia oryzae 24 hr after fungicide treatments. After inoculation the plants were kept in a dew room at 22° C. with 100% relative humidity for 48 hr to permit spores to germinate and infect the leaf. The plants were then transferred to a greenhouse set at 24° C. for disease to develop. Fungicide formulation, application and disease assessment on the sprayed leaves followed the procedures as described in the Example A.

### Example M: Evaluation of Fungicidal Activity: Tomato Early Blight (Alternaria solani; Bayer Code ALTESO)

Tomato plants (variety Outdoor Girl) were propagated in soil-less Metro mix, with each pot having one plant, and used when 12 to 14 days old. Test plants were inoculated with an aqueous spore suspension of Alternaria solani 24 hr after fungicide treatments. After inoculation the plants were kept in 100% relative humidity (one day in a dark dew chamber followed by two to three days in a lighted dew chamber at 20° C.) to permit spores to germinate and infect the leaf. The plants were then transferred to a growth room at 22° C. for disease to develop. Fungicide formulation, application and disease assessment on the sprayed leaves followed the procedures as described in the Example A.

### Example N: Evaluation of Fungicidal Activity: Cucumber Anthracnose (Glomerella lagenarium; Anamorph: Colletotrichum lagenarium; Bayer Code COLLLA)

Cucumber seedlings (variety Bush Pickle) were propagated in soil-less Metro mix, with each pot having one plant, and used in the test when 12 to 14 days old. Test plants were inoculated with an aqueous spore suspension of Colletotrichum lagenarium 24 hr after fungicide treatments. After inoculation the plants were kept in a dew room at 22° C. with 100% relative humidity for 48 hr to permit spores to germinate and infect the leaf. The plants were then transferred to a growth room set at 22° C. for disease to develop. Fungicide formulation, application and disease assessment on the sprayed leaves followed the procedures as described in the Example A.

TABLE 1
Compound Structure, Appearance, and Preparation Method

| | | Prepared | |
|---|---|---|---|
| Compound | | According to | |
| Number | Structure | Example | Appearance |
| 1 | 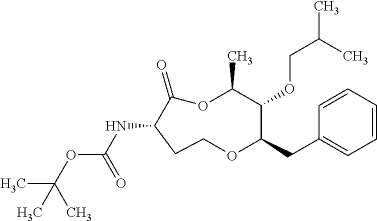 [SMILES: CC(C)CO[C@H]1[C@H](C)OC(=O)[C@@H](NC(=O)OC(C)(C)C)CCO[C@@H]1Cc1ccccc1] | Example 3; Example 4, Steps 1, 2, 3; Example 5, Steps 1, 2A; Example 6, Steps 1, 2, 3; Example 7, Steps 1, 2; Example 9; Example 10; Example 12C, Steps 1, 2. | Colorless Oil |
| 2 | 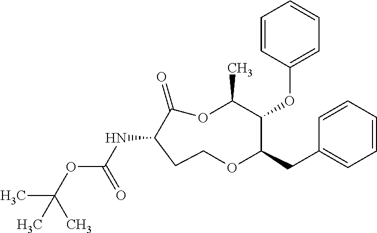 [SMILES: C[C@@H]1OC(=O)[C@@H](NC(=O)OC(C)(C)C)CCO[C@H](Cc2ccccc2)[C@H]1Oc1ccccc1] | Example 3; Example 4, Steps 1, 2, 3; Example 5, Steps 1, 2A Example 6, Steps 1, 2, 3; Example 7, Steps 1, 2; Example 9; Example 10; Example 12B. | Colorless Oil |
| 3 | 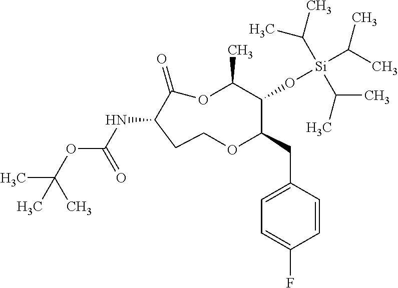 [SMILES: CC(C)[Si](O[C@H]1[C@H](C)OC(=O)[C@@H](NC(=O)OC(C)(C)C)CCO[C@@H]1Cc1ccc(F)cc1)(C(C)C)C(C)C] | Example 3; Example 4, Steps 1, 2, 3; Example 5, Steps 1, 2A Example 6, Steps 1, 2, 3; Example 7, Steps 1, 2; Example 9. | White Foam |
| 4 | 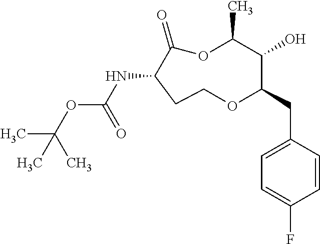 [SMILES: C[C@@H]1OC(=O)[C@@H](NC(=O)OC(C)(C)C)CCO[C@H](Cc2ccc(F)cc2)[C@H]1O] | Example 3; Example 4, Steps 1, 2, 3; Example 5, Steps 1, 2A Example 6, Steps 1, 2, 3; Example 7, Steps 1, 2; Example 9; Example 10. | White Solid |
| 5 | 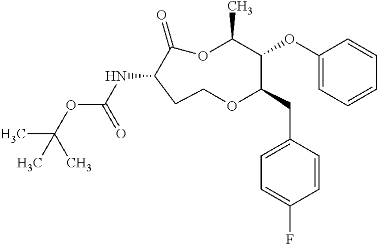 [SMILES: C[C@@H]1OC(=O)[C@@H](NC(=O)OC(C)(C)C)CCO[C@H](Cc2ccc(F)cc2)[C@H]1Oc1ccccc1] | Example 3; Example 4, Steps 1, 2, 3; Example 5, Steps 1, 2A Example 6, Steps 1, 2, 3; Example 7, Steps 1, 2; Example 9; Example 10; Example 12B. | White Foam |
| 6 | 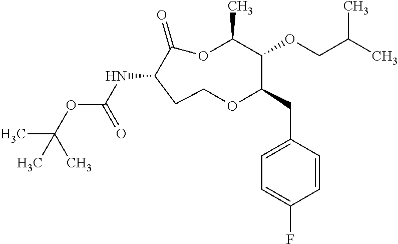 [SMILES: CC(C)CO[C@H]1[C@H](C)OC(=O)[C@@H](NC(=O)OC(C)(C)C)CCO[C@@H]1Cc1ccc(F)cc1] | Example 3; Example 4, Steps 1, 2, 3; Example 5, Steps 1, 2A Example 6, Steps 1, 2, 3; Example 7, Steps 1, 2; Example 9; Example 10; Example 12C, Steps 1, 2. | White Foam |
| 7 | 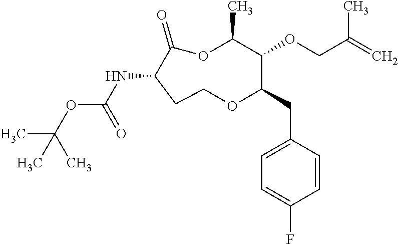 [SMILES: C=C(C)CO[C@H]1[C@H](C)OC(=O)[C@@H](NC(=O)OC(C)(C)C)CCO[C@@H]1Cc1ccc(F)cc1] | Example 3; Example 4, Steps 1, 2, 3; Example 5, Steps 1, 2A Example 6, Steps 1, 2, 3; Example 7, Steps 1, 2; Example 9; Example 10; Example 12C, Step 1. | Clear Tacky Solid |
| 8 | 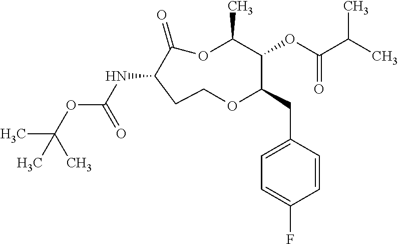 [SMILES: CC(C)C(=O)O[C@H]1[C@H](C)OC(=O)[C@@H](NC(=O)OC(C)(C)C)CCO[C@@H]1Cc1ccc(F)cc1] | Example 3; Example 4, Steps 1, 2, 3; Example 5, Steps 1, 2A Example 6, Steps 1, 2, 3; Example 7, Steps 1, 2; Example 9; Example 10; Example 12A. | White Foam |
| 9 | 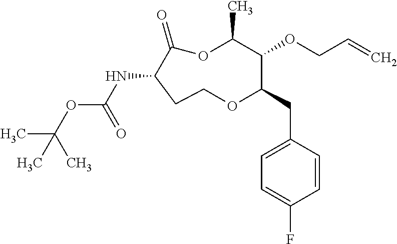 [SMILES: C=CCO[C@H]1[C@H](C)OC(=O)[C@@H](NC(=O)OC(C)(C)C)CCO[C@@H]1Cc1ccc(F)cc1] | Example 3; Example 4, Steps 1, 2, 3; Example 5, Steps 1, 2A Example 6, Steps 1, 2, 3; Example 7, Steps 1, 2; Example 9; Example 10; Example 12C, Step 1. | White Foam |
| 10 | 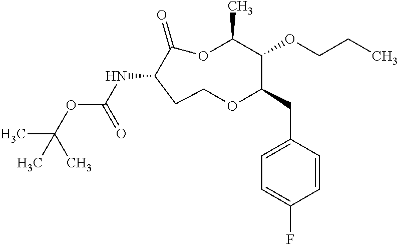 [SMILES: CCCO[C@H]1[C@H](C)OC(=O)[C@@H](NC(=O)OC(C)(C)C)CCO[C@@H]1Cc1ccc(F)cc1] | Example 3; Example 4, Steps 1, 2, 3; Example 5, Steps 1, 2A Example 6, Steps 1, 2, 3; Example 7, Steps 1, 2; Example 9; Example 10; Example 12C, Steps 1, 2. | White Foam |
| 11 | 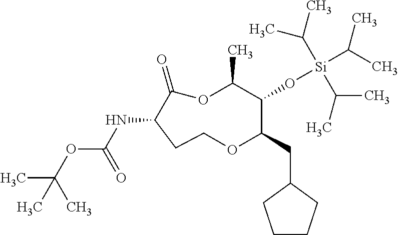 [SMILES: CC(C)[Si](O[C@H]1[C@H](C)OC(=O)[C@@H](NC(=O)OC(C)(C)C)CCO[C@@H]1CC1CCCC1)(C(C)C)C(C)C] | Example 3; Example 4, Steps 1, 2, 3; Example 5, Steps 1, 2A Example 6, Steps 1, 2, 3; Example 7, Steps 1, 2; Example 9. | White Powder |
| 12 | 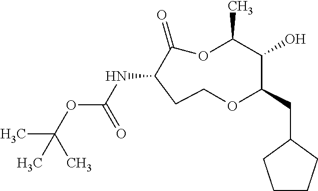 [SMILES: C[C@@H]1OC(=O)[C@@H](NC(=O)OC(C)(C)C)CCO[C@H](CC2CCCC2)[C@H]1O] | Example 3; Example 4, Steps 1, 2, 3; Example 5, Steps 1, 2A Example 6, Steps 1, 2, 3; Example 7, Steps 1, 2; Example 9; Example 10. | White Powder |
| 13 | 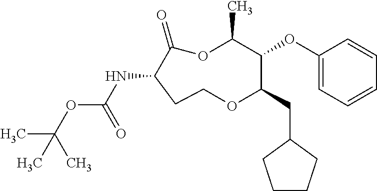 [SMILES: C[C@@H]1OC(=O)[C@@H](NC(=O)OC(C)(C)C)CCO[C@H](CC2CCCC2)[C@H]1Oc1ccccc1] | Example 3; Example 4, Steps 1, 2, 3; Example 5, Steps 1, 2A Example 6, Steps 1, 2, 3; Example 7, Steps 1, 2; Example 9; Example 10; Example 12B. | Colorless Film |
| 14 | 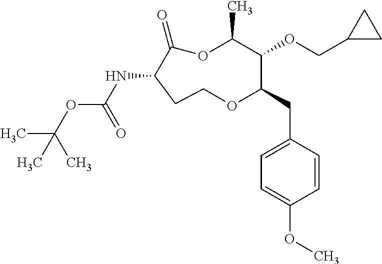 [SMILES: COc1ccc(C[C@H]2OCC[C@H](NC(=O)OC(C)(C)C)C(=O)O[C@@H](C)[C@@H]2OCC2CC2)cc1] | Example 3; Example 4, Steps 1, 2, 3; Example 5, Steps 1, 2B; Example 6, Steps 1, 2, 3; Example 7, Steps 1, 2; Example 8, Steps 1, 2; Example 9. | White Solid |
| 15 | 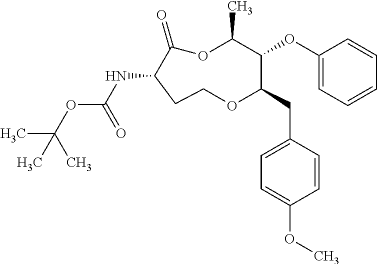 [SMILES: COc1ccc(C[C@H]2OCC[C@H](NC(=O)OC(C)(C)C)C(=O)O[C@@H](C)[C@@H]2Oc2ccccc2)cc1] | Example 3, Steps 1, 2; Example 4, Steps 1, 2, 3; Example 5, Steps 1, 2; Example 6, Steps 1, 2, 3; Example 7, Steps 1, 2; Example 8, Steps 1, 2; Example 9; Example 10; Example 12B. | White Solid |
| 16 | 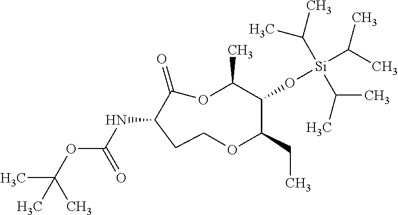 [SMILES: CC[C@H]1OCC[C@H](NC(=O)OC(C)(C)C)C(=O)O[C@@H](C)[C@@H]1O[Si](C(C)C)(C(C)C)C(C)C] | Example 3; Example 4, Steps 1, 2, 3; Example 5, Steps 1, 2A Example 6, Steps 1, 2, 3; Example 7, Steps 1, 2; Example 9. | White Foam |
| 17 | 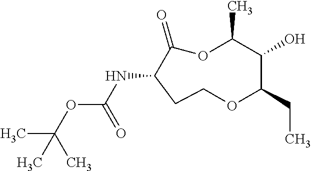 [SMILES: CC[C@H]1OCC[C@H](NC(=O)OC(C)(C)C)C(=O)O[C@@H](C)[C@@H]1O] | Example 3; Example 4, Steps 1, 2, 3; Example 5, Steps 1, 2A Example 6, Steps 1, 2, 3; Example 7, Steps 1, 2; Example 9; Example 10. | White Solid |
| 18 | 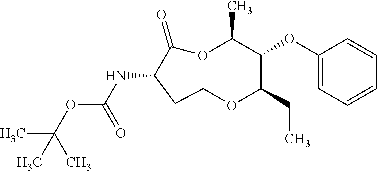 [SMILES: CC[C@H]1OCC[C@H](NC(=O)OC(C)(C)C)C(=O)O[C@@H](C)[C@@H]1Oc1ccccc1] | Example 3; Example 4, Steps 1, 2, 3; Example 5, Steps 1, 2A Example 6, Steps 1, 2, 3; Example 7, Steps 1, 2; Example 9; Example 10; Example 12B. | White Solid |
| 19 | 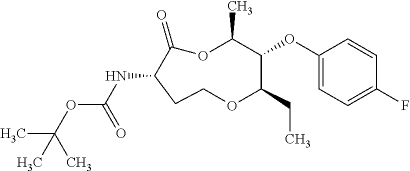 [SMILES: CC[C@H]1OCC[C@H](NC(=O)OC(C)(C)C)C(=O)O[C@@H](C)[C@@H]1Oc1ccc(F)cc1] | Example 3; Example 4, Steps 1, 2, 3; Example 5, Steps 1, 2A Example 6, Steps 1, 2, 3; Example 7, Steps 1, 2; Example 9; Example 10; Example 12B. | White Solid |
| 20 | 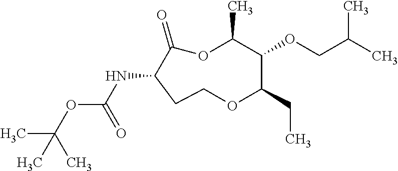 [SMILES: CC[C@H]1OCC[C@H](NC(=O)OC(C)(C)C)C(=O)O[C@@H](C)[C@@H]1OCC(C)C] | Example 3; Example 4, Steps 1, 2, 3; Example 5, Steps 1, 2A Example 6, Steps 1, 2, 3; Example 7, Steps 1, 2; Example 9; Example 10; Example 12C, Steps 1, 2. | White Foam |
| 21 | 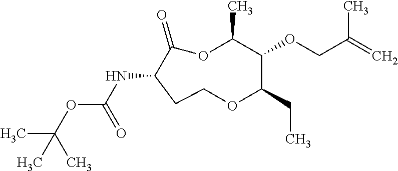 [SMILES: C=C(C)CO[C@H]1[C@H](C)OC(=O)[C@@H](NC(=O)OC(C)(C)C)CCO[C@@H]1CC] | Example 3; Example 4, Steps 1, 2, 3; Example 5, Steps 1, 2A Example 6, Steps 1, 2, 3; Example 7, Steps 1, 2; Example 9; Example 10; Example 12C, Step 1. | Tacky Oil |
| 22 | 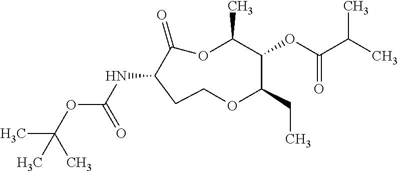 [SMILES: CC[C@H]1OCC[C@H](NC(=O)OC(C)(C)C)C(=O)O[C@@H](C)[C@@H]1OC(=O)C(C)C] | Example 3; Example 4, Steps 1, 2, 3; Example 5, Steps 1, 2A Example 6, Steps 1, 2, 3; Example 7, Steps 1, 2; Example 9; Example 10; Example 12A. | White Foam |
| 23 | 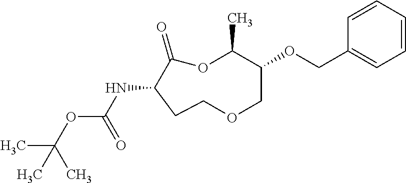 [SMILES: C[C@@H]1OC(=O)[C@@H](NC(=O)OC(C)(C)C)CCOC[C@H]1OCc1ccccc1] | Example 1, Steps 1, 2, 3, 4, 5; Example 2, Steps 1, 2; Example 6, Steps 1, 2, 3; Example 8, Steps 1, 2; Example 9. | Sticky Oil |
| 24 | 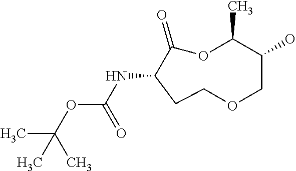 [SMILES: C[C@@H]1OC(=O)[C@@H](NC(=O)OC(C)(C)C)CCOC[C@H]1O] | Example 1, Steps 1, 2, 3, 4, 5; Example 2, Steps 1, 2; Example 6, Steps 1, 2, 3; Example 8, Steps 1, 2; Example 9; Example 11. | White Solid |
| 25 | 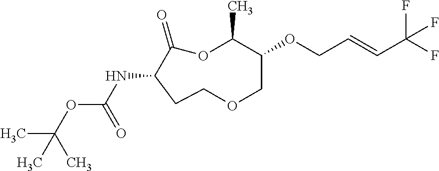 [SMILES: C[C@@H]1OC(=O)[C@@H](NC(=O)OC(C)(C)C)CCOC[C@H]1OC/C=C/C(F)(F)F] | Example 1, Steps 1, 2, 3, 4, 5; Example 2, Steps 1, 2; Example 6, Steps 1, 2, 3 Example 8, Steps 1, 2; Example 9; Example 11; Example 12C, Step 1. | Sticky Yellow Oil |
| 26 | 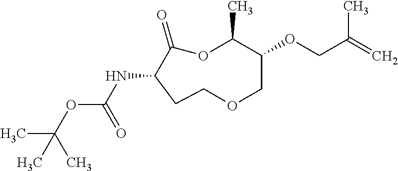 [SMILES: C=C(C)CO[C@@H]1COCC[C@H](NC(=O)OC(C)(C)C)C(=O)O[C@H]1C] | Example 1, Steps 1, 2, 3, 4, 5; Example 2, Steps 1, 2; Example 6, Steps 1, 2, 3 Example 8, Steps 1, 2; Example 9; Example 11; Example 12C, Step 1. | Sticky Oil |
| 27 | 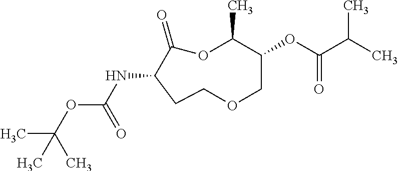 [SMILES: CC(C)C(=O)O[C@@H]1COCC[C@H](NC(=O)OC(C)(C)C)C(=O)O[C@H]1C] | Example 1, Steps 1, 2, 3, 4, 5; Example 2, Steps 1, 2; Example 6, Steps 1, 2, 3 Example 8, Steps 1, 2; Example 9; Example 11; Example 12A. | Hygroscopic Solid |
| 28 | 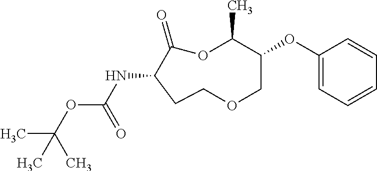 [SMILES: C[C@@H]1OC(=O)[C@@H](NC(=O)OC(C)(C)C)CCOC[C@H]1Oc1ccccc1] | Example 1, Steps 1, 2, 3, 4, 5; Example 2, Steps 1, 2; Example 6, Steps 1, 2, 3 Example 8, Steps 1, 2; Example 9; Example 11; Example 12B. | White Powder |
| 29 | 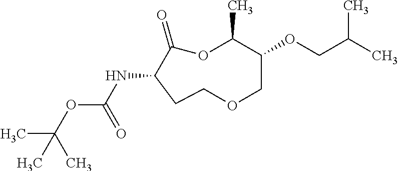 [SMILES: CC(C)CO[C@@H]1COCC[C@H](NC(=O)OC(C)(C)C)C(=O)O[C@H]1C] | Example 1, Steps 1, 2, 3, 4, 5; Example 2, Steps 1, 2; Example 6, Steps 1, 2, 3 Example 8, Steps 1, 2; Example 9; Example 11; Example 12C, Steps 1, 2. | White Solid |
| 30 | 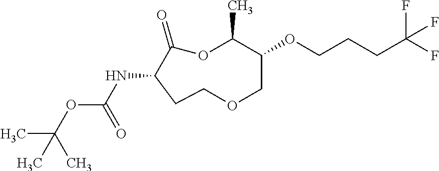 [SMILES: C[C@@H]1OC(=O)[C@@H](NC(=O)OC(C)(C)C)CCOC[C@H]1OCCCC(F)(F)F] | Example 1, Steps 1, 2, 3, 4, 5; Example 2, Steps 1, 2; Example 6, Steps 1, 2, 3 Example 8, Steps 1, 2; Example 9; Example 11; Example 12C, Steps 1, 2. | White Solid |
| 31 | 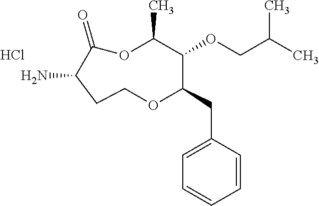 [SMILES: Cl ; CC(C)CO[C@H]1[C@H](C)OC(=O)[C@@H](N)CCO[C@@H]1Cc1ccccc1] | Example 13A, Step 1. | Oily Solid |
| 32 | 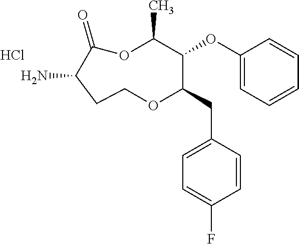 [SMILES: Cl ; C[C@@H]1OC(=O)[C@@H](N)CCO[C@H](Cc2ccc(F)cc2)[C@H]1Oc1ccccc1] | Example 13A, Step 1. | White Solid |
| 33 | 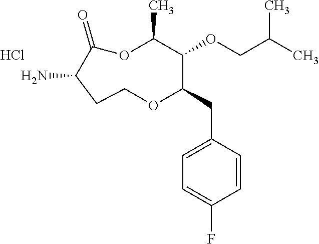 [SMILES: Cl ; CC(C)CO[C@H]1[C@H](C)OC(=O)[C@@H](N)CCO[C@@H]1Cc1ccc(F)cc1] | Example 13A, Step 1. | White Solid |
| 34 | 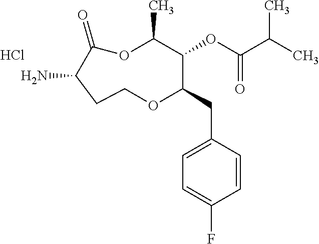 [SMILES: Cl ; CC(C)C(=O)O[C@H]1[C@H](C)OC(=O)[C@@H](N)CCO[C@@H]1Cc1ccc(F)cc1] | Example 13A, Step 1. | White Solid |
| 35 | 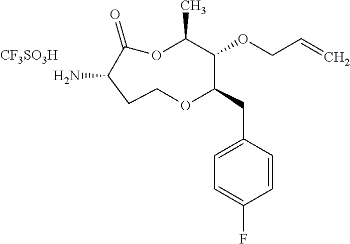 [SMILES: "CF3SO3H" ; C=CCO[C@H]1[C@H](C)OC(=O)[C@@H](N)CCO[C@@H]1Cc1ccc(F)cc1] | Example 13B, Step 1. | White Solid |
| 36 | 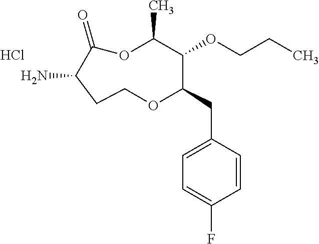 [SMILES: Cl ; CCCO[C@H]1[C@H](C)OC(=O)[C@@H](N)CCO[C@@H]1Cc1ccc(F)cc1] | Example 13A, Step 1. | White Solid |
| 37 | 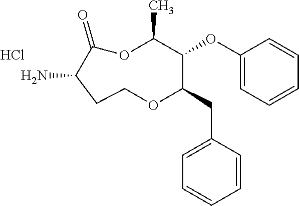 [SMILES: Cl ; C[C@@H]1OC(=O)[C@@H](N)CCO[C@H](Cc2ccccc2)[C@H]1Oc1ccccc1] | Example 13A, Step 1. | Yellow Oil |
| 38 | 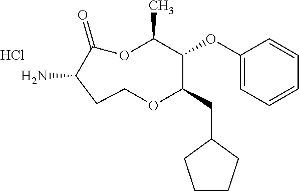 [SMILES: Cl ; C[C@@H]1OC(=O)[C@@H](N)CCO[C@H](CC2CCCC2)[C@H]1Oc1ccccc1] | Example 13A, Step 1. | White Solid |
| 39 | 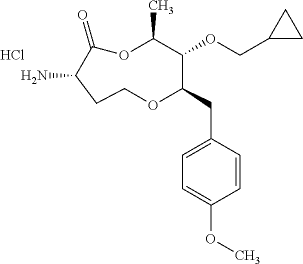 [SMILES: Cl ; COc1ccc(C[C@H]2OCC[C@H](N)C(=O)O[C@@H](C)[C@@H]2OCC2CC2)cc1] | Example 13A, Step 1. | Oil |
| 40 | 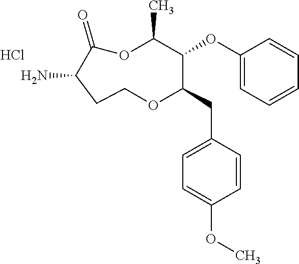 [SMILES: Cl ; COc1ccc(C[C@H]2OCC[C@H](N)C(=O)O[C@@H](C)[C@@H]2Oc2ccccc2)cc1] | Example 13A, Step 1. | White Solid |
| 41 | 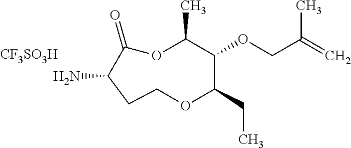 [SMILES: "CF3SO3H" ; C=C(C)CO[C@H]1[C@H](C)OC(=O)[C@@H](N)CCO[C@@H]1CC] | Example 13B, Step 1. | White Solid |
| 42 | 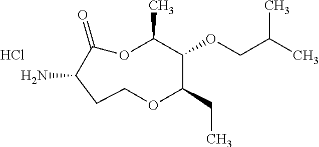 [SMILES: Cl ; CC[C@H]1OCC[C@H](N)C(=O)O[C@@H](C)[C@@H]1OCC(C)C] | Example 13A, Step 1. | White Solid |
| 43 | 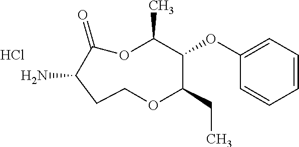 [SMILES: Cl ; CC[C@H]1OCC[C@H](N)C(=O)O[C@@H](C)[C@@H]1Oc1ccccc1] | Example 13A, Step 1. | White Solid |
| 44 | 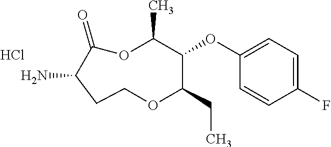 [SMILES: Cl ; CC[C@H]1OCC[C@H](N)C(=O)O[C@@H](C)[C@@H]1Oc1ccc(F)cc1] | Example 13A, Step 1. | White Solid |
| 45 | 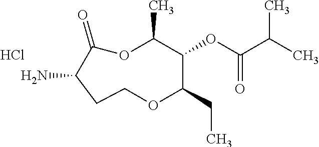 [SMILES: Cl ; CC[C@H]1OCC[C@H](N)C(=O)O[C@@H](C)[C@@H]1OC(=O)C(C)C] | Example 13A, Step 1. | White Solid |
| 46 | 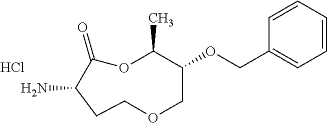 [SMILES: Cl ; C[C@@H]1OC(=O)[C@@H](N)CCOC[C@H]1OCc1ccccc1] | Example 13A, Step 1. | White Solid |
| 47 | 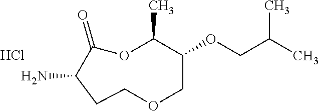 [SMILES: Cl ; CC(C)CO[C@@H]1COCC[C@H](N)C(=O)O[C@H]1C] | Example 13A, Step 1. | White Solid |
| 48 | 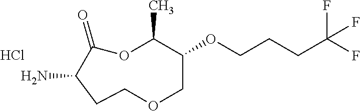 [SMILES: Cl ; C[C@@H]1OC(=O)[C@@H](N)CCOC[C@H]1OCCCC(F)(F)F] | Example 13A, Step 1. | White Solid |
| 49 | 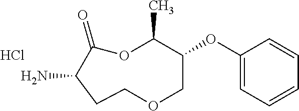 [SMILES: Cl ; C[C@@H]1OC(=O)[C@@H](N)CCOC[C@H]1Oc1ccccc1] | Example 13A, Step 1. | White Solid |
| 50 | 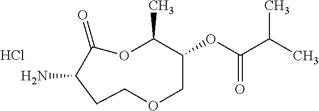 [SMILES: Cl ; CC(C)C(=O)O[C@@H]1COCC[C@H](N)C(=O)O[C@H]1C] | Example 13A, Step 1. | White Solid |
| 51 | 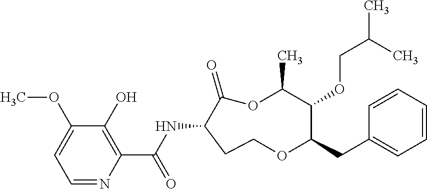 [SMILES: COc1ccnc(C(=O)N[C@H]2CCO[C@H](Cc3ccccc3)[C@@H](OCC(C)C)[C@H](C)OC2=O)c1O] | Example 13A, Step 2. | Soft White Solid |
| 52 | 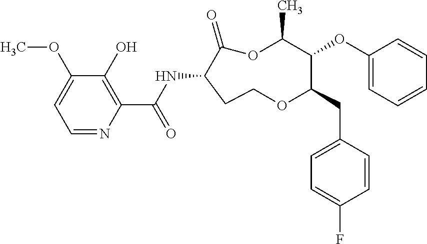 [SMILES: COc1ccnc(C(=O)N[C@H]2CCO[C@H](Cc3ccc(F)cc3)[C@@H](Oc3ccccc3)[C@H](C)OC2=O)c1O] | Example 13A, Step 2. | White Foam |
| 53 | 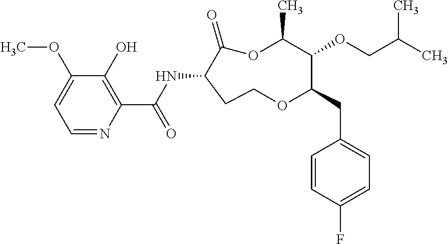 [SMILES: COc1ccnc(C(=O)N[C@H]2CCO[C@H](Cc3ccc(F)cc3)[C@@H](OCC(C)C)[C@H](C)OC2=O)c1O] | Example 13A, Step 2. | White Foam |
| 54 | 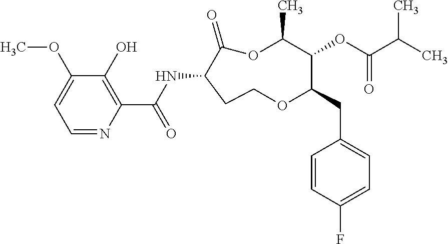 [SMILES: COc1ccnc(C(=O)N[C@H]2CCO[C@H](Cc3ccc(F)cc3)[C@@H](OC(=O)C(C)C)[C@H](C)OC2=O)c1O] | Example 13A, Step 2. | White Foam |
| 55 | 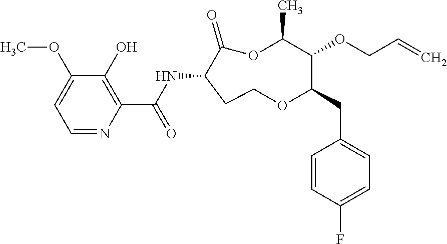 [SMILES: C=CCO[C@H]1[C@H](C)OC(=O)[C@@H](NC(=O)c2nccc(OC)c2O)CCO[C@@H]1Cc1ccc(F)cc1] | Example 13B, Step 2. | White Solid |
| 56 | 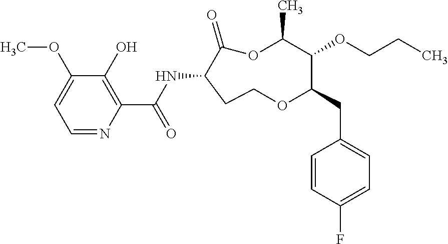 [SMILES: CCCO[C@H]1[C@H](C)OC(=O)[C@@H](NC(=O)c2nccc(OC)c2O)CCO[C@@H]1Cc1ccc(F)cc1] | Example 13A, Step 2. | White Solid |
| 57 | 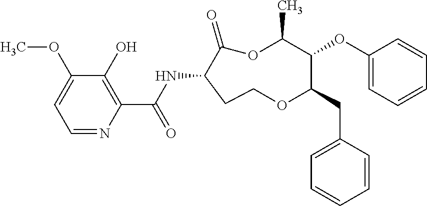 [SMILES: COc1ccnc(C(=O)N[C@H]2CCO[C@H](Cc3ccccc3)[C@@H](Oc3ccccc3)[C@H](C)OC2=O)c1O] | Example 13A, Step 2. | White Foam |
| 58 | 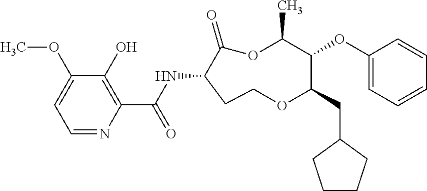 [SMILES: COc1ccnc(C(=O)N[C@H]2CCO[C@H](CC3CCCC3)[C@@H](Oc3ccccc3)[C@H](C)OC2=O)c1O] | Example 13A, Step 2. | White Powder |
| 59 | 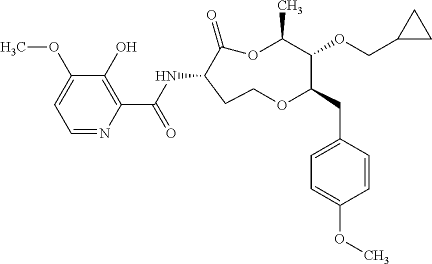 [SMILES: COc1ccc(C[C@H]2OCC[C@H](NC(=O)c3nccc(OC)c3O)C(=O)O[C@@H](C)[C@@H]2OCC2CC2)cc1] | Example 13A, Step 2. | White Solid |
| 60 | 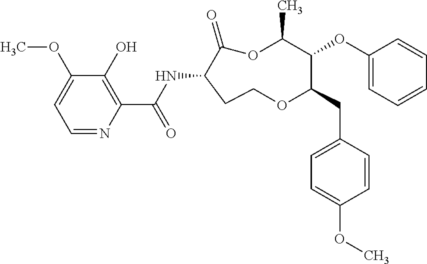 [SMILES: COc1ccc(C[C@H]2OCC[C@H](NC(=O)c3nccc(OC)c3O)C(=O)O[C@@H](C)[C@@H]2Oc2ccccc2)cc1] | Example 13A, Step 2. | White Solid |
| 61 | 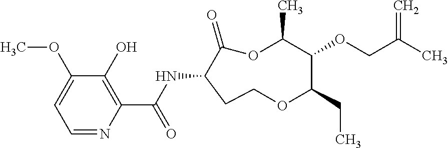 [SMILES: C=C(C)CO[C@H]1[C@H](C)OC(=O)[C@@H](NC(=O)c2nccc(OC)c2O)CCO[C@@H]1CC] | Example 13B, Step 2. | White Solid |
| 62 | 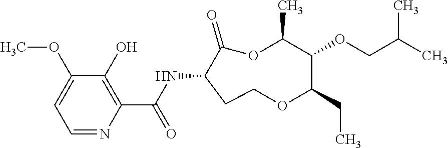 [SMILES: CC[C@H]1OCC[C@H](NC(=O)c2nccc(OC)c2O)C(=O)O[C@@H](C)[C@@H]1OCC(C)C] | Example 13A, Step 2. | White Solid |
| 63 | 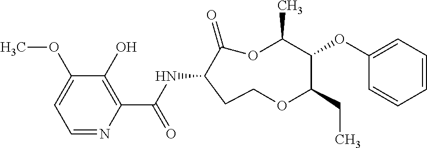 [SMILES: CC[C@H]1OCC[C@H](NC(=O)c2nccc(OC)c2O)C(=O)O[C@@H](C)[C@@H]1Oc1ccccc1] | Example 13A, Step 2. | White Solid |
| 64 | 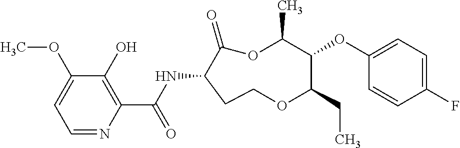 [SMILES: CC[C@H]1OCC[C@H](NC(=O)c2nccc(OC)c2O)C(=O)O[C@@H](C)[C@@H]1Oc1ccc(F)cc1] | Example 13A, Step 2. | White Solid |
| 65 | 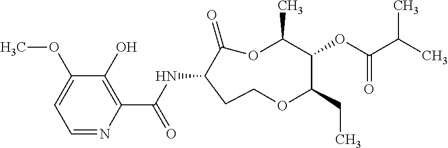 [SMILES: CC[C@H]1OCC[C@H](NC(=O)c2nccc(OC)c2O)C(=O)O[C@@H](C)[C@@H]1OC(=O)C(C)C] | Example 13A, Step 2. | White Foam |
| 66 | 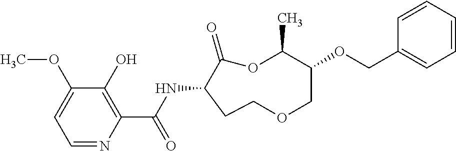 [SMILES: COc1ccnc(C(=O)N[C@H]2CCOC[C@@H](OCc3ccccc3)[C@H](C)OC2=O)c1O] | Example 13A, Step 2. | White Solid |
| 67 | 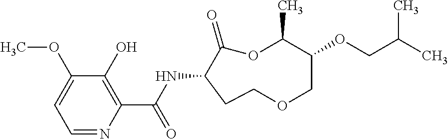 [SMILES: COc1ccnc(C(=O)N[C@H]2CCOC[C@@H](OCC(C)C)[C@H](C)OC2=O)c1O] | Example 13A, Step 2. | White Solid |
| 68 | 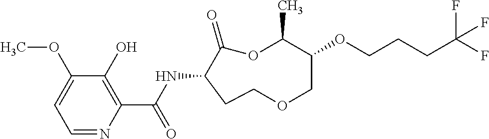 [SMILES: COc1ccnc(C(=O)N[C@H]2CCOC[C@@H](OCCCC(F)(F)F)[C@H](C)OC2=O)c1O] | Example 13A, Step 2. | White Solid |
| 69 | 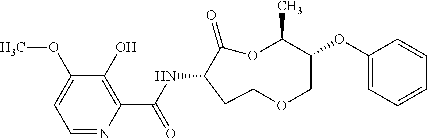 [SMILES: COc1ccnc(C(=O)N[C@H]2CCOC[C@@H](Oc3ccccc3)[C@H](C)OC2=O)c1O] | Example 13A, Step 2. | White Solid |
| 70 | 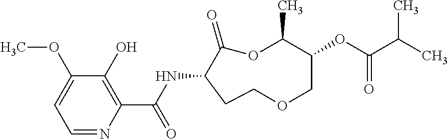 [SMILES: COc1ccnc(C(=O)N[C@H]2CCOC[C@@H](OC(=O)C(C)C)[C@H](C)OC2=O)c1O] | Example 13A, Step 2. | White Solid |
| 71 | 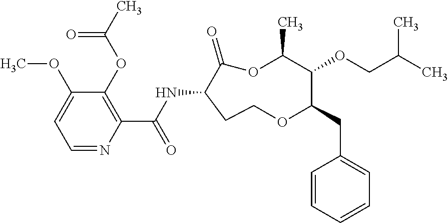 [SMILES: COc1ccnc(C(=O)N[C@H]2CCO[C@H](Cc3ccccc3)[C@@H](OCC(C)C)[C@H](C)OC2=O)c1OC(C)=O] | Example 14. | Off White Foam |
| 72 | 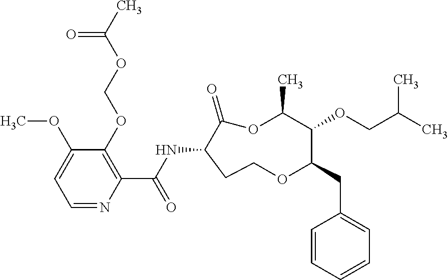 [SMILES: COc1ccnc(C(=O)N[C@H]2CCO[C@H](Cc3ccccc3)[C@@H](OCC(C)C)[C@H](C)OC2=O)c1OCOC(C)=O] | Example 15. | White Powder |
| 73 | 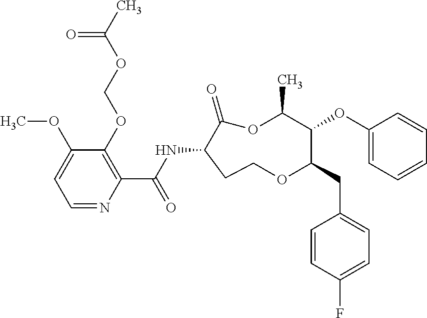 [SMILES: COc1ccnc(C(=O)N[C@H]2CCO[C@H](Cc3ccc(F)cc3)[C@@H](Oc3ccccc3)[C@H](C)OC2=O)c1OCOC(C)=O] | Example 15. | White Foam |
| 74 | 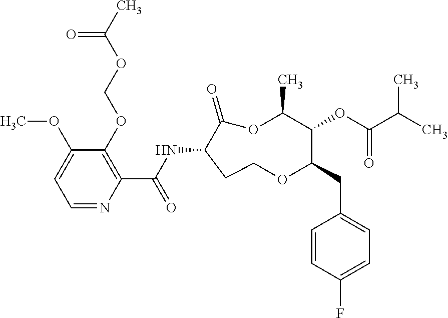 [SMILES: COc1ccnc(C(=O)N[C@H]2CCO[C@H](Cc3ccc(F)cc3)[C@@H](OC(=O)C(C)C)[C@H](C)OC2=O)c1OCOC(C)=O] | Example 15. | White Foam |
| 75 | 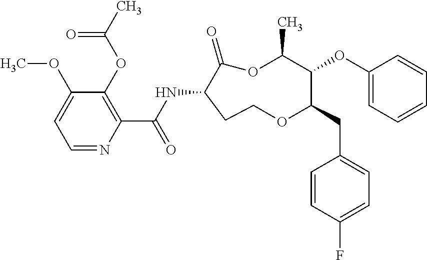 [SMILES: COc1ccnc(C(=O)N[C@H]2CCO[C@H](Cc3ccc(F)cc3)[C@@H](Oc3ccccc3)[C@H](C)OC2=O)c1OC(C)=O] | Example 14. | White Foam |
| 76 | 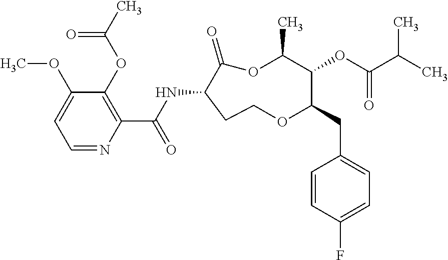 [SMILES: COc1ccnc(C(=O)N[C@H]2CCO[C@H](Cc3ccc(F)cc3)[C@@H](OC(=O)C(C)C)[C@H](C)OC2=O)c1OC(C)=O] | Example 14. | White Foam |
| 77 | 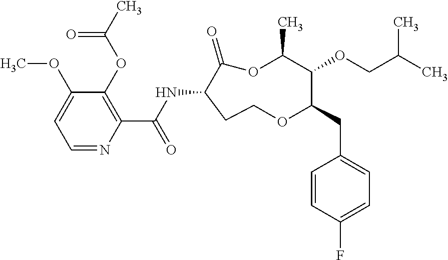 [SMILES: COc1ccnc(C(=O)N[C@H]2CCO[C@H](Cc3ccc(F)cc3)[C@@H](OCC(C)C)[C@H](C)OC2=O)c1OC(C)=O] | Example 14. | White Foam |
| 78 | 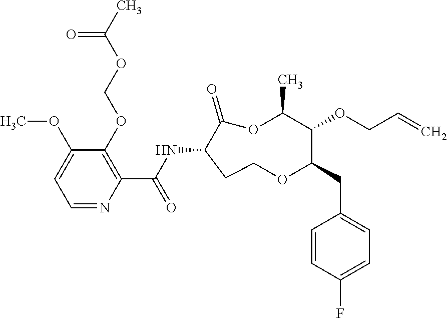 [SMILES: C=CCO[C@H]1[C@H](C)OC(=O)[C@@H](NC(=O)c2nccc(OC)c2OCOC(C)=O)CCO[C@@H]1Cc1ccc(F)cc1] | Example 15. | White Foam |
| 79 | 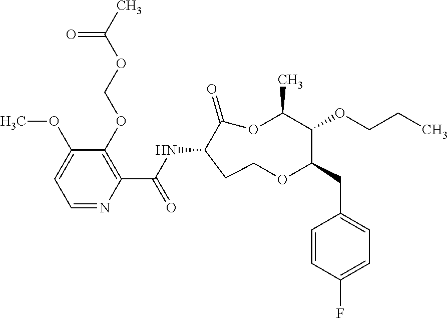 [SMILES: CCCO[C@H]1[C@H](C)OC(=O)[C@@H](NC(=O)c2nccc(OC)c2OCOC(C)=O)CCO[C@@H]1Cc1ccc(F)cc1] | Example 15. | White Foam |
| 80 | 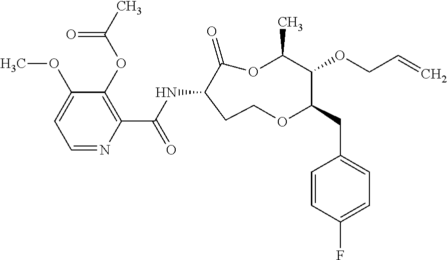 [SMILES: C=CCO[C@H]1[C@H](C)OC(=O)[C@@H](NC(=O)c2nccc(OC)c2OC(C)=O)CCO[C@@H]1Cc1ccc(F)cc1] | Example 14. | White Foam |
| 81 | 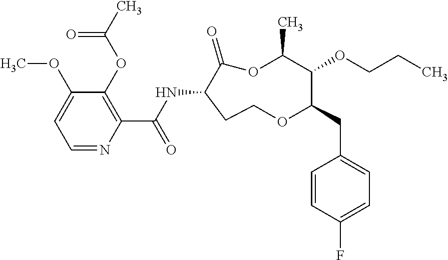 [SMILES: CCCO[C@H]1[C@H](C)OC(=O)[C@@H](NC(=O)c2nccc(OC)c2OC(C)=O)CCO[C@@H]1Cc1ccc(F)cc1] | Example 14. | White Solid |
| 82 | 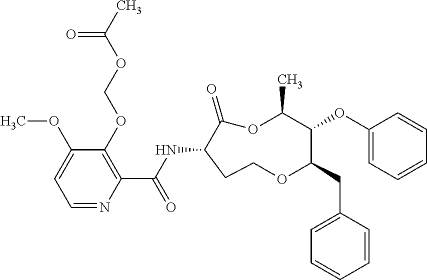 [SMILES: COc1ccnc(C(=O)N[C@H]2CCO[C@H](Cc3ccccc3)[C@@H](Oc3ccccc3)[C@H](C)OC2=O)c1OCOC(C)=O] | Example 15. | Off White Foam |
| 83 | 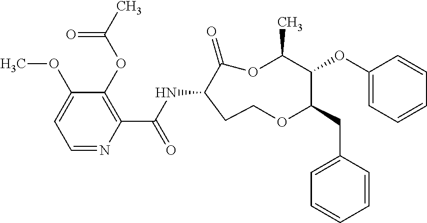 [SMILES: COc1ccnc(C(=O)N[C@H]2CCO[C@H](Cc3ccccc3)[C@@H](Oc3ccccc3)[C@H](C)OC2=O)c1OC(C)=O] | Example 14. | Light Orange Foam |
| 84 | 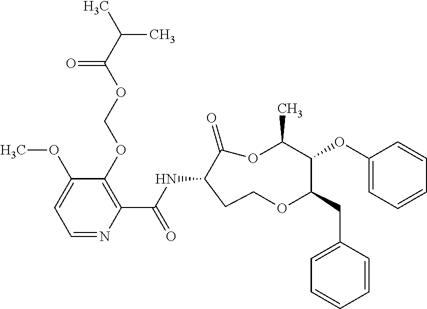 [SMILES: COc1ccnc(C(=O)N[C@H]2CCO[C@H](Cc3ccccc3)[C@@H](Oc3ccccc3)[C@H](C)OC2=O)c1OCOC(=O)C(C)C] | Example 15. | Off White Foam |
| 85 | 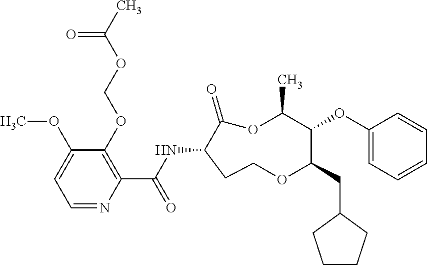 [SMILES: COc1ccnc(C(=O)N[C@H]2CCO[C@H](CC3CCCC3)[C@@H](Oc3ccccc3)[C@H](C)OC2=O)c1OCOC(C)=O] | Example 15. | White Powder |
| 86 | 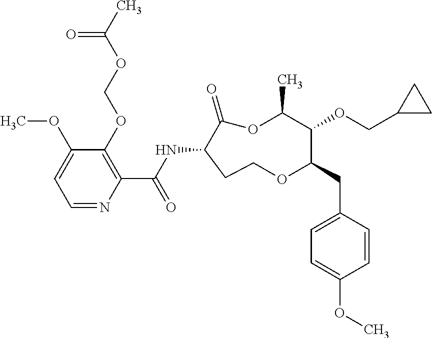 [SMILES: COc1ccc(C[C@H]2OCC[C@H](NC(=O)c3nccc(OC)c3OCOC(C)=O)C(=O)O[C@@H](C)[C@@H]2OCC2CC2)cc1] | Example 15. | White Solid |
| 87 | 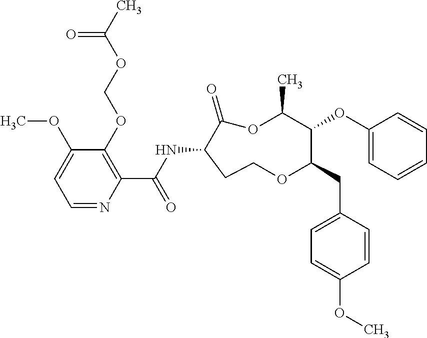 [SMILES: COc1ccc(C[C@H]2OCC[C@H](NC(=O)c3nccc(OC)c3OCOC(C)=O)C(=O)O[C@@H](C)[C@@H]2Oc2ccccc2)cc1] | Example 15. | White Solid |
| 88 | 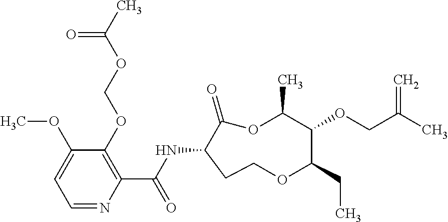 [SMILES: C=C(C)CO[C@H]1[C@H](C)OC(=O)[C@@H](NC(=O)c2nccc(OC)c2OCOC(C)=O)CCO[C@@H]1CC] | Example 15. | White Foam |
| 89 | 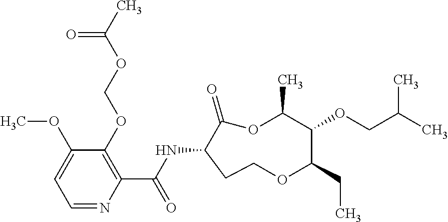 [SMILES: CC[C@H]1OCC[C@H](NC(=O)c2nccc(OC)c2OCOC(C)=O)C(=O)O[C@@H](C)[C@@H]1OCC(C)C] | Example 15. | White Foam |
| 90 | 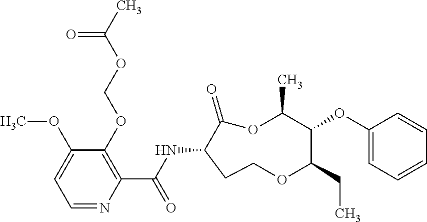 [SMILES: CC[C@H]1OCC[C@H](NC(=O)c2nccc(OC)c2OCOC(C)=O)C(=O)O[C@@H](C)[C@@H]1Oc1ccccc1] | Example 15. | White Foam |
| 91 | 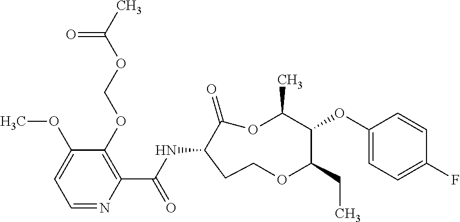 [SMILES: CC[C@H]1OCC[C@H](NC(=O)c2nccc(OC)c2OCOC(C)=O)C(=O)O[C@@H](C)[C@@H]1Oc1ccc(F)cc1] | Example 15. | White Foam |
| 92 | 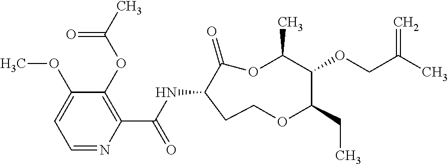 [SMILES: C=C(C)CO[C@H]1[C@H](C)OC(=O)[C@@H](NC(=O)c2nccc(OC)c2OC(C)=O)CCO[C@@H]1CC] | Example 14. | White Foam |
| 93 | 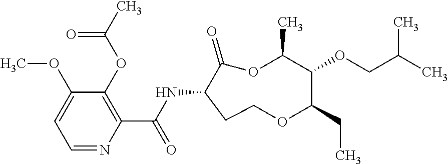 [SMILES: CC[C@H]1OCC[C@H](NC(=O)c2nccc(OC)c2OC(C)=O)C(=O)O[C@@H](C)[C@@H]1OCC(C)C] | Example 14. | White Foam |
| 94 | 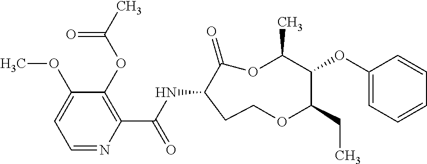 [SMILES: CC[C@H]1OCC[C@H](NC(=O)c2nccc(OC)c2OC(C)=O)C(=O)O[C@@H](C)[C@@H]1Oc1ccccc1] | Example 14. | White Foam |
| 95 | 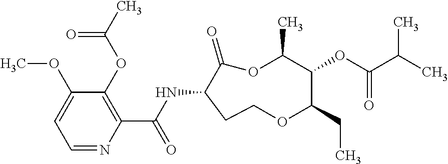 [SMILES: CC[C@H]1OCC[C@H](NC(=O)c2nccc(OC)c2OC(C)=O)C(=O)O[C@@H](C)[C@@H]1OC(=O)C(C)C] | Example 14. | White Foam |
| 96 | 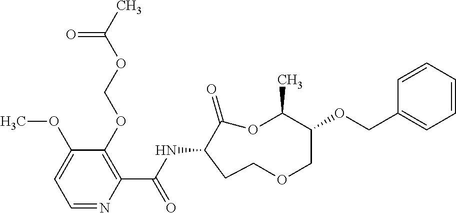 [SMILES: COc1ccnc(C(=O)N[C@H]2CCOC[C@@H](OCc3ccccc3)[C@H](C)OC2=O)c1OCOC(C)=O] | Example 15. | White Solid |
| 97 | 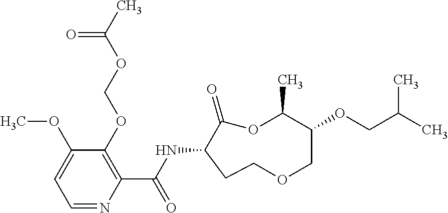 [SMILES: COc1ccnc(C(=O)N[C@H]2CCOC[C@@H](OCC(C)C)[C@H](C)OC2=O)c1OCOC(C)=O] | Example 15. | White Solid |
| 98 | 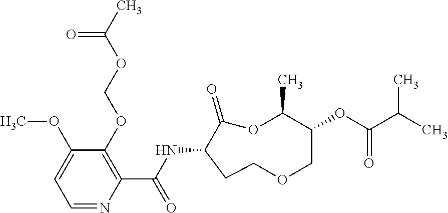 [SMILES: COc1ccnc(C(=O)N[C@H]2CCOC[C@@H](OC(=O)C(C)C)[C@H](C)OC2=O)c1OCOC(C)=O] | Example 15. | White Solid |
| 99 | 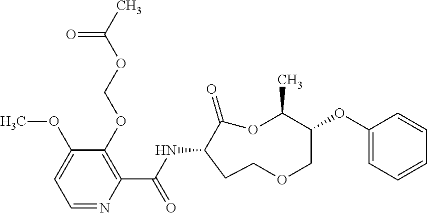 [SMILES: COc1ccnc(C(=O)N[C@H]2CCOC[C@@H](Oc3ccccc3)[C@H](C)OC2=O)c1OCOC(C)=O] | Example 15. | White Solid |
| 100 | 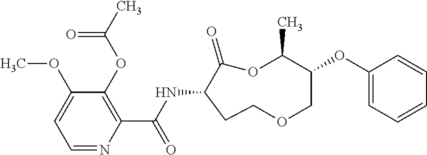 [SMILES: COc1ccnc(C(=O)N[C@H]2CCOC[C@@H](Oc3ccccc3)[C@H](C)OC2=O)c1OC(C)=O] | Example 14. | White Powder |
| 101 | 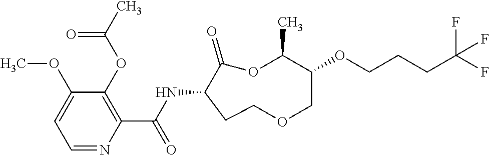 [SMILES: COc1ccnc(C(=O)N[C@H]2CCOC[C@@H](OCCCC(F)(F)F)[C@H](C)OC2=O)c1OC(C)=O] | Example 14. | White Powder |

TABLE 2
Analytical Data

| Cmpd. | MP | IR | | NMR |
|---|---|---|---|---|
| No. | (° C.) | (cm⁻¹) | MASS | (¹H, ¹³C or ¹⁹F) |
| 1 | — | — | ESIMS | ¹H NMR (400 MHz, CDCl₃) δ 7.38-7.06 (m, 5H), |
| | | | m/z 436 | 5.10 (d, J = 8.2 Hz, 1H), 5.07-5.00 (m, 1H), 4.22 (q, J = |
| | | | ([M + H]⁺) | 8.2 Hz, 1H), 3.82 (t, J = 9.3 Hz, 1H), 3.54-3.39 (m, |
| | | | | 3H), 3.31 (dd, J = 8.3, 6.5 Hz, 1H), 3.25 (t, J = 9.2 Hz, |
| | | | | 1H), 3.06 (d, J = 13.4 Hz, 1H), 2.81 (dd, J = 14.7, 11.4 |
| | | | | Hz, 1H), 2.15 (dt, J = 11.9, 5.5 Hz, 1H), 1.84 (dp, J = |
| | | | | 13.2, 6.6 Hz, 1H), 1.54-1.48 (m, 1H), 1.45 (d, J = 6.5 |
| | | | | Hz, 3H), 1.42 (s, 9H), 0.93 (d, J = 6.8 Hz, 6H) |
| | | | | ¹³C NMR (101 MHz, CDCl₃) δ 172.69, 154.91, 138.69, |
| | | | | 128.76, 128.45, 126.21, 82.41, 80.28, 80.22, 79.84, |
| | | | | 74.87, 60.80, 51.47, 35.91, 35.10, 29.14, 28.32, 19.42, |
| | | | | 19.40, 18.07 |
| 2 | — | — | ESIMS | ¹H NMR (400 MHz, CDCl₃) δ 7.35-7.15 (m, 7H), |
| | | | m/z 478 | 6.98 (m, 3H), 5.31-5.20 (m, 1H), 5.15 (d, J = 8.0 Hz, |
| | | | ([M + Na]⁺) | 1H), 4.45 (t, J = 9.2 Hz, 1H), 4.28 (q, J = 8.1 Hz, 1H), |
| | | | | 4.10-4.03 (m, 1H), 3.59 (t, J = 9.7 Hz, 1H), 3.48 (bs, |
| | | | | 1H), 2.95 (d, J = 13.4 Hz, 1H), 2.80 (dd, J = 14.7, 11.4 |
| | | | | Hz, 1H), 2.20 (dt, J = 11.7, 5.7 Hz, 1H), 1.55 (dtd, J = |
| | | | | 14.2, 9.9, 2.1 Hz, 1H), 1.42 (d, J = 2.1 Hz, 9H), 1.37 (d, |
| | | | | J = 6.5 Hz, 3H) |
| | | | | ¹³C NMR (101 MHz, CDCl₃) δ 172.67, 158.61, 154.91, |
| | | | | 138.13, 129.79, 128.74, 128.43, 126.29, 121.69, 115.62, |
| | | | | 82.08, 79.93, 77.52, 74.30, 61.26, 51.51, 36.24, 35.08, |
| | | | | 28.33, 18.27 |
| 3 | — | — | ESIMS | ¹H NMR (400 MHz, CDCl₃) δ 7.20 (dd, J = 8.5, 5.5 Hz, |
| | | | m/z 576 | 2H), 6.98 (t, J = 8.7 Hz, 2H), 5.11 (d, J = 8.2 Hz, 1H), |
| | | | ([M + Na]⁺) | 5.09-4.90 (m, 1H), 4.23 (q, J = 8.2 Hz, 1H), 3.89 (t, J = |
| | | | | 8.7 Hz, 1H), 3.75 (ddd, J = 11.3, 8.5, 2.2 Hz, 1H), |
| | | | | 3.48 (t, J = 9.9 Hz, 1H), 3.43-3.32 (m, 1H), 3.08 (d, J = |
| | | | | 14.8 Hz, 1H), 2.80 (dd, J = 15.0, 11.7 Hz, 1H), 2.15 |
| | | | | (dt, J = 13.1, 6.0 Hz, 1H), 1.49-1.38 (m, 4H), 1.42 (s, |
| | | | | 9H), 1.14 (s, 18H), 1.11-1.02 (m, 3H) |
| | | | | ¹⁹F NMR (376 MHz, CDCl₃) δ-116.96 |
| 4 | — | — | ESIMS | ¹H NMR (400 MHz, CDCl₃) δ 7.20 (dd, J = 8.5, 5.5 Hz, |
| | | | m/z 420 | 2H), 6.96 (t, J = 8.7 Hz, 2H), 5.17 (d, J = 8.2 Hz, 1H), |
| | | | ([M + Na]⁺) | 5.04 (dq, J = 13.0, 6.7 Hz, 1H), 4.22 (q, J = 8.3 Hz, 1H), |
| | | | | 3.75 (ddd, J = 10.5, 9.1, 3.0 Hz, 1H), 3.59 (td, J = 9.2, |
| | | | | 6.8 Hz, 1H), 3.51 (t, J = 9.8 Hz, 1H), 3.47-3.42 (m, |
| | | | | 1H), 3.14 (dd, J = 15.2, 2.9 Hz, 1H), 2.84 (dd, J = 15.2, |
| | | | | 10.6 Hz, 1H), 2.53 (d, J = 6.9 Hz, 1H), 2.25-2.10 (m, |
| | | | | 1H), 1.51 (dtd, J = 14.4, 10.2, 2.4 Hz, 1H), 1.42 (d, J = |
| | | | | 6.6 Hz, 3H), 1.42 (s, 9H) |
| | | | | ¹⁹F NMR (376 MHz, CDCl₃) δ-116.91 |
| 5 | — | — | ESIMS | ¹H NMR (400 MHz, CDCl₃) δ 7.33-7.27 (m, 2H), |
| | | | m/z 496 | 7.15 (dd, J = 8.5, 5.5 Hz, 2H), 7.02-6.90 (m, 5H), 5.33- |
| | | | ([M + Na]⁺) | 5.19 (m, 1H), 5.17 (d, J = 8.1 Hz, 1H), 4.44 (t, J = 9.2 |
| | | | | Hz, 1H), 4.29 (q, J = 8.1 Hz, 1H), 4.01 (td, J = 10.3, 9.1, |
| | | | | 2.8 Hz, 1H), 3.58 (t, J = 9.8 Hz, 1H), 3.50-3.38 (m, |
| | | | | 1H), 2.96-2.87 (m, 1H), 2.78 (dd, J = 15.1, 11.1 Hz, |
| | | | | 1H), 2.25-2.18 (m, 1H), 1.86-1.71 (m, 1H), 1.43 (s, |
| | | | | 9H), 1.36 (d, J = 6.5 Hz, 3H) |
| | | | | ¹⁹F NMR (376 MHz, CDCl₃) δ-116.86 |
| 6 | — | — | ESIMS | ¹H NMR (400 MHz, CDCl₃) δ 7.20 (dd, J = 8.5, 5.5 Hz, |
| | | | m/z 476 | 2H), 6.97 (t, J = 8.7 Hz, 2H), 5.14 (d, J = 8.1 Hz, 1H), |
| | | | ([M + Na]⁺) | 5.10-4.95 (m, 1H), 4.22 (q, J = 8.3 Hz, 1H), 3.76 (ddd, |
| | | | | J = 11.7, 9.2, 2.5 Hz, 1H), 3.54-3.44 (m, 2H), 3.43- |
| | | | | 3.35 (m, 1H), 3.31 (dd, J = 8.4, 6.4 Hz, 1H), 3.24 (t, J = |
| | | | | 9.2 Hz, 1H), 3.01 (d, J = 15.1 Hz, 1H), 2.79 (dd, J = |
| | | | | 14.9, 11.2 Hz, 1H), 2.20-2.10 (m, 1H), 1.89-1.76 (m, |
| | | | | 1H), 1.58-1.47 (m, 1H), 1.45 (d, J = 6.6 Hz, 3H), 1.42 |
| | | | | (s, 9H), 0.93 (dd, J = 6.7, 1.3 Hz, 6H) |
| | | | | ¹⁹F NMR (376 MHz, CDCl₃) δ-117.04 |
| 7 | — | — | ESIMS | ¹H NMR (400 MHz, CDCl₃) δ 7.20 (dd, J = 8.5, 5.5 Hz, |
| | | | m/z 474 | 2H), 6.97 (t, J = 8.7 Hz, 2H), 5.13 (d, J = 8.1 Hz, 1H), |
| | | | ([M + Na]⁺) | 5.10-5.02 (m, 1H), 5.00 (s, 1H), 4.89 (s, 1H), 4.23 (q, |
| | | | | J = 8.2 Hz, 1H), 4.13 (d, J = 11.6 Hz, 1H), 3.96 (d, J = |
| | | | | 11.6 Hz, 1H), 3.80 (ddd, J = 11.7, 9.3, 2.4 Hz, 1H), 3.49 |
| | | | | (t, J = 9.8 Hz, 1H), 3.45-3.36 (m, 1H), 3.34 (t, J = 9.2 |
| | | | | Hz, 1H), 3.03 (d, J = 15.3 Hz, 1H), 2.80 (dd, J = 14.9, |
| | | | | 11.3 Hz, 1H), 2.16 (dt, J = 13.2, 5.9 Hz, 1H), 1.75 (s, |
| | | | | 3H), 1.61-1.50 (m, 1H), 1.47 (d, J = 6.4 Hz, 3H), 1.42 |
| | | | | (s, 9H) |
| | | | | ¹⁹F NMR (376 MHz, CDCl₃) δ-116.99 |
| 8 | — | — | ESIMS | ¹H NMR (400 MHz, CDCl₃) δ 7.15 (dd, J = 8.6, 5.4 Hz, |
| | | | m/z 490 | 2H), 6.97 (t, J = 8.7 Hz, 2H), 5.27-5.17 (m, 1H), 5.14 |
| | | | ([M + Na]⁺) | (t, J = 9.4 Hz, 1H), 5.08 (d, J = 8.2 Hz, 1H), 4.27 (q, J = |
| | | | | 8.4 Hz, 1H), 3.95 (ddd, J = 10.6, 9.3, 3.3 Hz, 1H), 3.62 |
| | | | | (t, J = 10.1 Hz, 1H), 3.50-3.42 (m, 1H), 2.87 (dd, J = |
| | | | | 15.4, 10.7 Hz, 1H), 2.60 (dd, J = 15.2, 3.1 Hz, 1H), 2.50 |
| | | | | (hept, J = 7.0 Hz, 1H), 2.22 (dt, J = 14.9, 7.0 Hz, 1H), |
| | | | | 1.59-1.47 (m, 1H), 1.43 (s, 9H), 1.27 (d, J = 6.3 Hz, |
| | | | | 3H), 1.18 (d, J = 7.0 Hz, 3H), 1.17 (d, J = 7.0 Hz, 3H) |
| | | | | ¹⁹F NMR (376 MHz, CDCl₃) δ-116.62 |
| 9 | — | — | ESIMS | ¹H NMR (400 MHz, CDCl₃) δ 7.20 (dd, J = 8.6, 5.5 Hz, |
| | | | m/z 460 | 2H), 6.97 (t, J = 8.7 Hz, 2H), 5.89 (ddt, J = 17.2, 10.4, |
| | | | ([M + Na]⁺) | 5.6 Hz, 1H), 5.30 (dq, J = 17.2, 1.6 Hz, 1H), 5.21 (dq, J = |
| | | | | 10.3, 1.3 Hz, 1H), 5.10 (d, J = 8.2 Hz, 1H), 5.07- |
| | | | | 5.01 (m, 1H), 4.28-4.18 (m, 2H), 4.08 (ddt, J = 12.2, |
| | | | | 5.7, 1.4 Hz, 1H), 3.85-3.74 (m, 1H), 3.48 (t, J = 9.8 |
| | | | | Hz, 1H), 3.36 (q, J = 9.2, 8.6 Hz, 2H), 3.05 (d, J = 14.8 |
| | | | | Hz, 1H), 2.79 (dd, J = 14.9, 11.2 Hz, 1H), 2.21-2.10 |
| | | | | (m, 1H), 1.57-1.48 (m, 1H), 1.47 (d, J = 6.7 Hz, 3H), |
| | | | | 1.42 (s, 9H) |
| | | | | ¹⁹F NMR (376 MHz, CDCl₃) δ-117.01 |
| 10 | — | — | ESIMS | ¹H NMR (400 MHz, CDCl₃) δ 7.20 (dd, J = 8.5, 5.5 Hz, |
| | | | m/z 440 | 2H), 6.97 (t, J = 8.7 Hz, 2H), 5.13 (d, J = 8.2 Hz, 1H), |
| | | | ([M + H]⁺) | 5.07-4.97 (m, 1H), 4.22 (q, J = 8.3 Hz, 1H), 3.76 (ddd, |
| | | | | J = 11.6, 9.2, 2.6 Hz, 1H), 3.66 (dt, J = 8.6, 6.6 Hz, 1H), |
| | | | | 3.57-3.44 (m, 2H), 3.43-3.34 (m, 1H), 3.26 (t, J = |
| | | | | 9.2 Hz, 1H), 3.02 (dd, J = 14.9, 2.5 Hz, 1H), 2.80 (dd, |
| | | | | J = 14.9, 11.2 Hz, 1H), 2.23-2.09 (m, 1H), 1.66-1.52 |
| | | | | (m, 2H), 1.54-1.47 (m, 1H), 1.46 (d, J = 7.1 Hz, 3H), |
| | | | | 1.42 (s, 9H), 0.94 (t, J = 7.4 Hz, 3H) |
| | | | | ¹⁹F NMR (376 MHz, CDCl₃) δ-117.06 |
| 11 | — | — | ESIMS | ¹H NMR (400 MHz, CDCl₃) δ 5.17 (d, J = 8.3 Hz, 1H), |
| | | | m/z 551 | 4.99 (dq, J = 8.8, 6.6 Hz, 1H), 4.21 (dt, J = 10.5, 8.0 Hz, |
| | | | ([M + Na]⁺) | 1H), 3.74 (app t, J = 8.8 Hz, 1H), 3.58-3.41 (m, 2H), |
| | | | | 2.29-2.16 (m, 1H), 1.98-1.41 (m, 20H), 1.38 (d, J = |
| | | | | 6.6 Hz, 3H), 1.13-1.08 (m, 23H) |
| | | | | ¹³C NMR (101 MHz, CDCl₃) δ 173.08, 154.94, 81.80, |
| | | | | 79.72, 75.35, 74.16, 59.47, 51.47, 36.03, 35.33, 34.98, |
| | | | | 33.72, 31.37, 28.31, 24.99, 24.93, 18.41, 18.32, 18.29, |
| | | | | 14.00 |
| 12 | — | — | ESIMS | ¹H NMR (400 MHz, CDCl₃) δ 5.22-5.10 (m, 1H), |
| | | | m/z 373 | 5.04 (dq, J = 8.5, 6.3 Hz, 1H), 4.27-4.15 (m, 1H), 3.60- |
| | | | ([M + H]⁺) | 3.37 (m, 4H), 2.28-2.06 (m, 2H), 2.00-1.50 (m, |
| | | | | 10H), 1.44 (s, 9H), 1.40 (d, J = 6.4 Hz, 3H), 1.20-0.99 |
| | | | | (m, 2H) |
| | | | | ¹³C NMR (101 MHz, CDCl₃) δ 173.00, 155.02, 80.99, |
| | | | | 79.96, 74.32, 72.43, 59.38, 51.44, 36.01, 35.66, 34.97, |
| | | | | 33.46, 31.90, 28.32, 25.02, 24.96, 17.83 |
| 13 | — | — | ESIMS | ¹H NMR (400 MHz, CDCl₃) δ 7.33-7.24 (m, 2H), |
| | | | m/z 449 | 7.02-6.89 (m, 3H), 5.24 (dq, J = 9.4, 6.6 Hz, 1H), 5.16 |
| | | | ([M + H]⁺) | (d, J = 8.4 Hz, 1H), 4.32-4.21 (m, 2H), 3.73 (ddd, J = |
| | | | | 11.6, 9.3, 2.3 Hz, 1H), 3.64-3.53 (m, 2H), 2.33-2.23 |
| | | | | (m, 1H), 2.02-1.26 (m, 22H), 1.10-0.95 (m, 2H) |
| | | | | ¹³C NMR (101 MHz, CDCl₃) δ 172.94, 158.82, 154.96, |
| | | | | 129.69, 121.48, 115.70, 79.92, 79.76, 77.39, 74.26, |
| | | | | 59.59, 51.49, 35.98, 35.53, 35.11, 33.34, 31.79, 28.34, |
| | | | | 24.96, 24.91, 18.21 |
| 14 | — | — | HRMS-ESI | ¹H NMR (500 MHz, CDCl₃) δ 7.17 (d, J = 8.6 Hz, 2H), |
| | | | (m/z) | 6.83 (d, J = 8.7 Hz, 2H), 5.09 (d, J = 7.9 Hz, 1H), 5.03 |
| | | | ([M + Na]⁺) | (s, 1H), 4.22 (q, J = 8.1 Hz, 1H), 3.80 (s, 1H), 3.78 (s, |
| | | | calcd for | 3H), 3.51 (dd, J = 9.7, 7.1 Hz, 1H), 3.46 (d, J = 9.6 Hz, |
| | | | C₂₅H₃₇NNaO₇, | 1H), 3.44-3.35 (m, 2H), 3.27 (t, J = 9.2 Hz, 1H), 3.06 |
| | | | 486.2462; | (d, J = 14.8 Hz, 1H), 2.75 (dd, J = 14.5, 11.5 Hz, 1H), |
| | | | found, | 2.22-2.08 (m, 1H), 1.54-1.45 (m, 4H), 1.42 (s, 9H), |
| | | | 486.2441 | 1.07 (ttt, J = 9.7, 7.0, 4.9 Hz, 1H), 0.57 (dd, J = 8.1, 1.4 |
| | | | | Hz, 2H), 0.28-0.16(m, 2H) |
| | | | | ¹³C NMR (126 MHz, CDCl₃) δ 172.63, 158.05, 154.90, |
| | | | | 130.48, 129.69, 113.90, 80.37, 79.87, 78.41, 74.93, |
| | | | | 55.26, 51.49, 35.15, 28.32, 18.03, 11.05, 3.19, 3.14 |
| 15 | — | — | ESIMS | ¹H NMR (400 MHz, CDCl₃) δ 7.35-7.26 (m, 2H), |
| | | | m/z 486.5 | 7.16-7.08 (m, 2H), 7.04-6.93 (m, 3H), 6.85-6.76 |
| | | | ([M + H]⁺) | (m, 2H), 5.35-5.20 (m, 1H), 5.17 (d, J = 8.0 Hz, 1H), |
| | | | | 4.43 (t, J = 9.1 Hz, 1H), 4.28 (q, J = 8.1 Hz, 1H), 4.01 |
| | | | | (t, J = 9.1 Hz, 1H), 3.76 (s, 3H), 3.66-3.52 (m, 1H), |
| | | | | 3.46 (s, 1H), 2.89 (d, J = 13.5 Hz, 1H), 2.74 (dd, J = |
| | | | | 14.7, 11.4 Hz, 1H), 2.29-2.12 (m, 1H), 1.56 (dtd, J = |
| | | | | 14.1, 9.8, 2.0 Hz, 1H), 1.43 (s, 9H), 1.36 (d, J = 6.5 Hz, |
| | | | | 3H) |
| | | | | ¹³C NMR (101 MHz, CDCl₃) δ 172.67, 158.63, 158.08, |
| | | | | 154.92, 130.08, 129.78, 129.65, 121.66, 115.62, 113.88, |
| | | | | 82.26, 79.92, 74.31, 61.31, 55.22, 51.52, 35.35, 35.09, |
| | | | | 28.34, 18.27 |
| 16 | — | — | ESIMS | ¹H NMR (600 MHz, CDCl₃) δ 5.17 (d, J = 8.2 Hz, 1H), |
| | | | m/z 496 | 5.03-4.95 (m, 1H), 4.22 (q, J = 8.5 Hz, 1H), 3.73 (t, |
| | | | ([M + Na]⁺) | J = 8.8 Hz, 1H), 3.53 (dd, J = 9.8, 3.4 Hz, 1H), 3.45 (t, |
| | | | | J = 10.3 Hz, 1H), 3.28 (t, J = 9.9 Hz, 1H), 2.28-2.19 (m, |
| | | | | 1H), 1.77-1.65 (m, 1H), 1.65-1.52 (m, 2H), 1.44 (s, |
| | | | | 9H), 1.38 (d, J = 6.5 Hz, 3H), 1.10 (d, J = 5.2 Hz, 21H), |
| | | | | 0.97 (t, J = 7.3 Hz, 3H) |
| | | | | ¹³C NMR (151 MHz, CDCl₃) δ 173.07, 154.95, 84.51, |
| | | | | 79.77, 75.17, 74.26, 59.15, 51.51, 35.00, 28.33, 22.30, |
| | | | | 18.45, 18.28, 18.26, 13.90, 10.66 |
| 17 | — | — | ESIMS | ¹H NMR (500 MHz, CDCl₃) δ 5.15 (d, J = 8.4 Hz, 1H), |
| | | | m/z 318 | 5.04 (dq, J = 9.1, 6.4 Hz, 1H), 4.22 (dt, J = 10.0, 7.7 Hz, |
| | | | ([M + H]⁺) | 1H), 3.57-3.43 (m, 3H), 3.30 (ddd, J = 11.6, 9.2, 2.6 |
| | | | | Hz, 1H), 2.25 (ddd, J = 14.1, 7.2, 3.9 Hz, 1H), 1.87- |
| | | | | 1.75 (m, 2H), 1.65-1.54 (m, 2H), 1.44 (s, 9H), 1.40 (d, |
| | | | | J = 6.5 Hz, 3H), 0.99 (t, J = 7.3 Hz, 3H) |
| | | | | ¹³C NMR (126 MHz, CDCl₃) δ 172.97, 154.99, 83.36, |
| | | | | 79.94, 74.19, 72.56, 59.07, 51.45, 34.99, 28.33, 22.59, |
| | | | | 17.79, 10.44 |
| 18 | — | — | ESIMS | ¹H NMR (400 MHz, CDCl₃) δ 7.33-7.27 (m, 2H), |
| | | | m/z 394 | 7.00-6.88 (m, 3H), 5.28-5.21 (m, 1H), 5.18 (d, J = |
| | | | ([M + H]⁺) | 8.3 Hz, 1H), 4.39-4.22 (m, 2H), 3.66-3.52 (m, 3H), |
| | | | | 2.30 (dd, J = 14.6, 7.4 Hz, 1H), 1.78-1.50 (m, 3H), |
| | | | | 1.45 (s, 9H), 1.30 (d, J = 6.6 Hz, 3H), 0.94 (t, J = 7.3 |
| | | | | Hz, 3H) |
| | | | | ¹³C NMR (101 MHz, CDCl₃) δ 173.03, 158.82, 155.08, |
| | | | | 129.81, 121.60, 115.62, 82.32, 82.32, 80.05, 74.29, |
| | | | | 59.41, 51.62, 35.24, 28.46, 22.87, 18.26, 10.52 |
| 19 | — | — | ESIMS | ¹H NMR (400 MHz, CDCl₃) δ 7.01-6.94 (m, 2H), |
| | | | m/z 412 | 6.90-6.83 (m, 2H), 5.28-5.11 (m, 2H), 4.27 (dt, J = |
| | | | ([M + H]⁺) | 9.9, 7.7 Hz, 1H), 4.20 (t, J = 9.2 Hz, 1H), 3.70-3.48 |
| | | | | (m, 3H), 2.34-2.22 (m, 1H), 1.80-1.52 (m, 3H), 1.44 |
| | | | | (s, 9H), 1.30 (d, J = 6.6 Hz, 3H), 0.95 (t, J = 7.3 Hz, |
| | | | | 3H) |
| | | | | ¹⁹F NMR (471 MHz, CDCl₃) δ-122.84 |
| 20 | — | — | ESIMS | ¹H NMR (400 MHz, CDCl₃) δ 5.17 (d, J = 8.3 Hz, 1H), |
| | | | m/z 374 | 5.04 (dq, J = 9.0, 6.5 Hz, 1H), 4.21 (dt, J = 10.0, 7.7 Hz, |
| | | | ([M + H]⁺) | 1H), 3.56-3.43 (m, 2H), 3.40 (dd, J = 8.3, 6.6 Hz, 1H), |
| | | | | 3.32 (ddd, J = 11.5, 9.3, 2.5 Hz, 1H), 3.23 (dd, J = 8.4, |
| | | | | 6.3 Hz, 1H), 3.10 (t, J = 9.3 Hz, 1H), 2.24 (ddd, J = |
| | | | | 13.7, 6.8, 3.7 Hz, 1H), 1.88-1.73 (m, 1H), 1.73-1.63 |
| | | | | (m, 1H), 1.63-1.49 (m, 2H), 1.44 (s, 9H), 1.39 (d, J = |
| | | | | 6.6 Hz, 3H), 0.97 (t, J = 7.3 Hz, 3H), 0.90 (d, J = 6.7 |
| | | | | Hz, 3H), 0.89 (d, J = 6.7 Hz, 3H) |
| | | | | ¹³C NMR (101 MHz, CDCl₃) δ 172.96, 154.96, 82.55, |
| | | | | 80.14, 80.04, 79.78, 74.77, 58.88, 51.45, 35.11, 29.05, |
| | | | | 28.31, 22.32, 19.39, 19.33, 17.89, 10.48 |
| 21 | — | — | ESIMS | ¹H NMR (400 MHz, CDCl₃) δ 5.14 (d, J = 8.3 Hz, 1H), |
| | | | m/z 372 | 5.11-5.02 (m, 1H), 4.96 (dd, J = 2.1, 1.1 Hz, 1H), 4.87 |
| | | | ([M + H]⁺) | (ddd, J = 2.4, 1.6, 0.9 Hz, 1H), 4.22 (q, J = 8.5 Hz, 1H), |
| | | | | 4.05 (d, J = 11.5 Hz, 1H), 3.89 (d, J = 11.5 Hz, 1H), |
| | | | | 3.59-3.42 (m, 2H), 3.37 (ddd, J = 11.5, 9.4, 2.6 Hz, |
| | | | | 1H), 3.18 (t, J = 9.3 Hz, 1H), 2.30-2.17 (m, 1H), 1.73 |
| | | | | (s, 3H), 1.71-1.47 (m, 3H), 1.44 (s, 9H), 1.41 (d, J = |
| | | | | 6.5 Hz, 3H), 0.97 (t, J = 7.3 Hz, 3H) |
| | | | | ¹³C NMR (101 MHz, CDCl₃) δ 173.10, 155.12, 141.76, |
| | | | | 112.49, 82.60, 80.66, 80.15, 80.02, 74.78, 59.15, 51.62, |
| | | | | 35.31, 28.48, 22.58, 19.89, 18.04, 10.64 |
| 22 | — | — | ESIMS | ¹H NMR (400 MHz, CDCl₃) δ 5.23-5.08 (m, 2H), |
| | | | m/z 388 | 5.00 (t, J = 9.6 Hz, 1H), 4.26 (dt, J = 10.2, 7.8 Hz, 1H), |
| | | | ([M + H]⁺) | 3.67-3.51 (m, 2H), 3.46 (ddd, J = 11.7, 9.7, 2.7 Hz, |
| | | | | 1H), 2.55 (h, J = 7.0 Hz, 1H), 2.33-2.20 (m, 1H), 1.68- |
| | | | | 1.49 (m, 2H), 1.44 (s, 9H), 1.35-1.25 (m, 1H), 1.23 |
| | | | | (d, J = 6.5 Hz, 3H), 1.18 (d, J = 7.0 Hz, 3H), 1.17 (d, |
| | | | | J = 7.0 Hz, 3H), 0.94 (t, J = 7.4 Hz, 3H) |
| | | | | ¹³C NMR (101 MHz, CDCl₃) δ 175.91, 172.95, 154.90, |
| | | | | 80.68, 79.87, 72.60, 71.19, 58.86, 51.37, 34.85, 34.08, |
| | | | | 28.29, 22.08, 18.88, 18.84, 17.26, 10.22 |
| 23 | — | — | ESIMS | ¹H NMR (400 MHz, CDCl₃) δ 7.37-7.28 (m, 5H), |
| | | | m/z 381 | 5.32 (d, J = 7.3 Hz, 1H), 5.23 (p, J = 6.6 Hz, 1H), 4.65- |
| | | | ([M + H]⁺) | 4.51 (m, 2H), 4.28 (q, J = 7.2 Hz, 1H), 3.86-3.78 (m, |
| | | | | 1H), 3.73 (dd, J = 11.8, 5.6 Hz, 1H), 3.58 (dd, J = 11.8, |
| | | | | 3.6 Hz, 1H), 3.52-3.38 (m, 2H), 2.40-2.24 (m, 1H), |
| | | | | 1.78-1.68 (m, 1H), 1.44 (s, 9H), 1.35 (d, J = 6.7 Hz, |
| | | | | 3H) |
| | | | | ¹³C NMR (101 MHz, CDCl₃) δ 172.89, 155.04, 137.75, |
| | | | | 128.45, 127.89, 127.85, 79.82, 78.38, 73.48, 72.22, |
| | | | | 68.57, 66.20, 51.31, 33.15, 28.33, 18.92 |
| 24 | 148- | — | ESIMS | ¹H NMR (400 MHz, CDCl₃) δ 5.39-5.29 (m, 1H), |
| | 152 | | m/z 312 | 5.25 (d, J = 7.7 Hz, 1H), 4.35-4.24 (m, 1H), 4.07 (ddd, |
| | | | ([M + Na]⁺) | J = 10.9, 5.9, 2.8 Hz, 1H), 3.95 (ddd, J = 11.2, 4.3, 1.1 |
| | | | | Hz, 1H), 3.50-3.39 (m, 3H), 3.12 (d, J = 11.1 Hz, 1H), |
| | | | | 2.33-2.21 (m, 1H), 1.80-1.66 (m, 1H), 1.44 (s, 9H), |
| | | | | 1.32 (d, J = 6.9 Hz, 3H) |
| 25 | — | — | ESIMS | ¹H NMR (400 MHz, CDCl₃) δ 6.44-6.33 (m, 1H), |
| | | | m/z 420 | 6.00-5.86 (m, 1H), 5.33 (d, J = 7.3 Hz, 1H), 5.27- |
| | | | ([M + Na]⁺) | 5.16 (m, 1H), 4.30 (q, J = 7.0 Hz, 1H), 4.26-4.06 (m, |
| | | | | 2H), 3.87 (ddd, J = 10.6, 7.6, 2.6 Hz, 1H), 3.76 (dd, J = |
| | | | | 11.9, 5.0 Hz, 1H), 3.60 (dd, J = 12.0, 3.4 Hz, 1H), 3.51- |
| | | | | 3.38 (m, 2H), 2.41-2.30 (m, 1H), 1.75 (dtd, J = 15.0, |
| | | | | 7.7, 2.6 Hz, 1H), 1.44 (s, 9H), 1.39 (d, J = 6.7 Hz, 3H) |
| | | | | 19F NMR (376 MHz, CDCl₃) d -64.32 |
| 26 | — | — | ESIMS | ¹H NMR (400 MHz, CDCl₃) δ 5.32 (d, J = 7.5 Hz, 1H), |
| | | | m/z 366 | 5.18 (p, J = 6.6 Hz, 1H), 4.99-4.92 (m, 1H), 4.92- |
| | | | ([M + Na]⁺) | 4.86 (m, 1H), 4.33-4.23 (m, 1H), 4.00-3.90 (m, 2H), |
| | | | | 3.83 (ddd, J = 10.8, 7.1, 2.6 Hz, 1H), 3.70 (dd, J = 11.8, |
| | | | | 5.8 Hz, 1H), 3.61 (dd, J = 11.8, 3.7 Hz, 1H), 3.51-3.37 |
| | | | | (m, 2H), 2.39-2.27 (m, 1H), 1.77-1.67 (m, 4H), 1.44 |
| | | | | (s, 9H), 1.38 (d, J = 6.6 Hz, 3H) |
| | | | | ¹³C NMR (101 MHz, CDCl₃) δ 172.88, 155.03, 141.80, |
| | | | | 113.04, 79.78, 78.29, 74.41, 73.53, 68.64, 66.15, 51.31, |
| | | | | 33.21, 28.31, 19.47, 18.92 |
| 27 | — | — | ESIMS | ¹H NMR (400 MHz, CDCl₃) δ 5.29 (d, J = 7.6 Hz, 1H), |
| | | | m/z 360 | 5.27-5.16 (m, 1H), 4.86-4.69 (m, 1H), 4.38-4.22 |
| | | | ([M + H]⁺) | (m, 1H), 3.92 (ddd, J = 10.8, 6.6, 2.6 Hz, 1H), 3.73 (dd, |
| | | | | J = 12.0, 5.2 Hz, 1H), 3.65 (dd, J = 12.1, 3.4 Hz, 1H), |
| | | | | 3.49 (ddd, J = 10.9, 8.7, 2.1 Hz, 1H), 2.60 (app hept, J = |
| | | | | 7.0 Hz, 1H), 2.39-2.23 (m, 1H), 1.73 (dtd, J = 14.4, |
| | | | | 8.6, 2.6 Hz, 1H), 1.44 (s, 9H), 1.36 (d, J = 6.7 Hz, 3H), |
| | | | | 1.20 (d, J = 7.0 Hz, 3H), 1.16 (d, J = 6.9 Hz, 3H) |
| | | | | ¹³C NMR (101 MHz, CDCl₃) δ 176.69, 172.59, 154.99, |
| | | | | 79.85, 72.94, 72.52, 68.55, 67.16, 51.33, 33.81, 33.24, |
| | | | | 28.30, 18.99, 18.73, 18.43 |
| 28 | — | — | ESIMS | ¹H NMR (400 MHz, CDCl₃) δ 7.32-7.23 (m, 2H), |
| | | | m/z 388 | 7.02-6.94 (m, 1H), 6.94-6.87 (m, 2H), 5.41-5.22 |
| | | | ([M + Na]⁺) | (m, 2H), 4.39-4.27 (m, 2H), 3.92-3.72 (m, 3H), 3.53 |
| | | | | (ddd, J = 10.7, 8.6, 2.1 Hz, 1H), 2.41-2.29 (m, 1H), |
| | | | | 1.83-1.68 (m, 1H), 1.46-1.40 (m, 12H) |
| | | | | ¹³C NMR (101 MHz, CDCl₃) δ 172.86, 157.67, 155.03, |
| | | | | 129.66, 121.80, 116.20, 79.87, 77.32, 73.01, 68.95, |
| | | | | 66.56, 51.38, 33.46, 28.33, 18.75 |
| 29 | 99- | — | ESIMS | ¹H NMR (400 MHz, CDCl₃) δ 5.38-5.26 (m, 1H), |
| | 103 | | m/z 346 | 5.18-5.07 (m, 1H), 4.27 (q, J = 7.2 Hz, 1H), 3.79 (ddd, |
| | | | ([M + H]⁺) | J = 10.8, 6.9, 2.6 Hz, 1H), 3.68-3.59 (m, 2H), 3.48 |
| | | | | (ddd, J = 10.7, 8.3, 2.1 Hz, 1H), 3.37 (dt, J = 7.3, 5.2 |
| | | | | Hz, 1H), 3.29 (dd, J = 8.8, 6.2 Hz, 1H), 3.18 (dd, J = |
| | | | | 8.8, 6.9 Hz, 1H), 2.37-2.27 (m, 1H), 1.89-1.74 (m, |
| | | | | 1H), 1.71 (dtd, J = 14.5, 8.3, 2.6 Hz, 1H), 1.44 (s, 9H), |
| | | | | 1.39 (d, J = 6.6 Hz, 3H), 0.90 (d, J = 6.7 Hz, 3H), 0.88 |
| | | | | (d, J = 6.7 Hz, 3H) |
| 30 | 80- | — | ESIMS | ¹H NMR (400 MHz, CDCl₃) δ 5.33 (d, J = 7.3 Hz, 1H), |
| | 83 | | m/z 401 | 5.15 (p, J = 6.6 Hz, 1H), 4.29 (td, J = 7.7, 5.7 Hz, 1H), |
| | | | ([M + H]⁺) | 3.84 (ddd, J = 11.1, 7.4, 2.6 Hz, 1H), 3.70 (dd, J = 11.9, |
| | | | | 5.3 Hz, 1H), 3.65-3.53 (m, 2H), 3.53-3.41 (m, 2H), |
| | | | | 3.36 (ddd, J = 6.5, 5.2, 3.5 Hz, 1H), 2.41-2.28 (m, |
| | | | | 1H), 2.26-2.10 (m, 2H), 1.88-1.66 (m, 3H), 1.44 (s, |
| | | | | 9H), 1.38 (d, J = 6.7 Hz, 3H) |
| 31 | — | — | ESIMS | ¹H NMR (400 MHz, CDCl₃) δ 8.53 (bs, 3H), 7.26- |
| | | | m/z 336 | 6.94 (m, 5H), 4.94 (bs, 1H), 3.98 (m, 1H), 3.81 (m, 1H), |
| | | | ([M + H]⁺) | 3.58-3.16 (m, 5H), 3.02 (d, J = 13.6 Hz, 1H), 2.76 (m, |
| | | | | 1H), 2.33 (m, 1H), 1.95-1.78 (m, 2H), 1.43 (s, 3H), |
| | | | | 0.93 (d, J = 6.5 Hz, 6H) |
| | | | | ¹³C NMR (101 MHz, CDCl₃) δ 169.73, 138.55, 128.81, |
| | | | | 128.50, 126.26, 82.51, 80.26, 79.91, 76.20, 67.10, |
| | | | | 51.04, 35.89, 32.17, 29.14, 19.44, 19.40, 17.94 |
| 32 | — | — | ESIMS | — |
| | | | m/z 374 | |
| | | | ([M + H]⁺) | |
| 33 | — | — | ESIMS | — |
| | | | m/z 354 | |
| | | | ([M + H]⁺) | |
| 34 | — | — | ESIMS | — |
| | | | m/z 368 | |
| | | | ([M + H]⁺) | |
| 35 | — | — | ESIMS | — |
| | | | m/z 338 | |
| | | | ([M + H]⁺) | |
| 36 | — | — | ESIMS | — |
| | | | m/z 340 | |
| | | | ([M + H]⁺) | |
| 37 | — | — | ESIMS | ¹H NMR (400 MHz, DMSO-d₆) δ 8.27 (bs, 3H), 7.39- |
| | | | m/z 356 | 7.14 (m, 9H), 7.00 (t, J = 7.3 Hz, 1H), 5.24-5.12 (m, |
| | | | ([M + H]⁺) | 1H), 4.73 (t, J = 9.2 Hz, 1H), 4.03-3.91 (m, 2H), 3.78 |
| | | | | (t, J = 10.3 Hz, 1H), 3.55 (bd, J = 9.3 Hz, 1H), 3.06- |
| | | | | 2.96 (m, 1H), 2.75-2.65 (m, 1H), 2.15 (m, 1H), 1.57 |
| | | | | (m, 1H), 1.28 (d, J = 6.5 Hz, 3H) |
| 38 | — | — | ESIMS | — |
| | | | m/z 348 | |
| | | | ([M + H]⁺) | |
| 39 | — | — | ESIMS | — |
| | | | m/z 364.4 | |
| | | | ([M + H]⁺) | |
| 40 | — | — | ESIMS | — |
| | | | m/z 386.3 | |
| | | | ([M + H]⁺) | |
| 41 | — | — | ESIMS | — |
| | | | m/z 272 | |
| | | | ([M + H]⁺) | |
| 42 | — | — | ESIMS | — |
| | | | m/z 274 | |
| | | | ([M + H]⁺) | |
| 43 | — | — | ESIMS | — |
| | | | m/z 294 | |
| | | | ([M + H]⁺) | |
| 44 | — | — | ESIMS | — |
| | | | m/z 312 | |
| | | | ([M + H]⁺) | |
| 45 | — | — | ESIMS | — |
| | | | m/z 288 | |
| | | | ([M + H]⁺) | |
| 46 | — | — | ESIMS | — |
| | | | m/z 280 | |
| | | | ([M + H]⁺) | |
| 47 | — | — | ESIMS | — |
| | | | m/z 246 | |
| | | | ([M + H]⁺) | |
| 48 | — | — | ESIMS | — |
| | | | m/z 300 | |
| | | | ([M + H]⁺) | |
| 49 | — | — | ESIMS | — |
| | | | m/z 266 | |
| | | | ([M + H]⁺) | |
| 50 | — | — | ESIMS | — |
| | | | m/z 260 | |
| | | | ([M + H]⁺) | |
| 51 | — | — | ESIMS | ¹H NMR (400 MHz, CDCl₃) δ 12.07 (d, J = 0.5 Hz, |
| | | | m/z 487 | 1H), 8.52 (d, J = 8.0 Hz, 1H), 7.97 (d, J = 5.1 Hz, 1H), |
| | | | ([M + H]⁺) | 7.36-7.12 (m, 5H), 6.85 (d, J = 5.1 Hz, 1H), 5.16- |
| | | | | 5.03 (m, 1H), 4.65-4.57 (m, 1H), 3.93 (s, 3H), 3.91- |
| | | | | 3.83 (m, 1H), 3.60-3.44 (m, 3H), 3.37-3.27 (m, 2H), |
| | | | | 3.10 (dd, J = 14.8, 2.3 Hz, 1H), 2.84 (dd, J = 14.8, 11.4 |
| | | | | Hz, 1H), 2.28 (dt, J = 12.5, 5.5 Hz, 1H), 1.85 (dp, J = |
| | | | | 13.2, 6.5 Hz, 1H), 1.75-1.62 (m, 1H), 1.49 (d, J = 6.5 |
| | | | | Hz, 3H), 0.94 (d, J = 6.8 Hz, 6H) |
| | | | | ¹³C NMR (101 MHz, CDCl₃) δ 171.54, 168.68, 155.29, |
| | | | | 148.67, 140.57, 138.64, 130.38, 128.79, 128.47, 126.24, |
| | | | | 109.45, 82.76, 80.24, 75.37, 61.08, 56.06, 50.12, 36.09, |
| | | | | 34.48, 29.15, 19.42, 19.41, 18.10 |
| 52 | — | — | HRMS-ESI | ¹H NMR (400 MHz, CDCl₃) δ 12.04 (s, 1H), 8.56 (d, J = |
| | | | (m/z) | 7.9 Hz, 1H), 7.98 (d, J = 5.2 Hz, 1H), 7.32 (dd, J = |
| | | | ([M + H]⁺) | 8.7, 7.3 Hz, 2H), 7.17 (dd, J = 8.5, 5.5 Hz, 2H), 7.04- |
| | | | calcd for | 6.98 (m, 3H), 6.95 (t, J = 8.7 Hz, 2H), 6.86 (d, J = 5.2 |
| | | | C₂₈H₃₀FN₂O₇, | Hz, 1H), 5.37-5.20 (m, 1H), 4.68 (td, J = 8.3, 7.0 Hz, |
| | | | 525.2032; | 1H), 4.50 (t, J = 9.1 Hz, 1H), 4.06 (ddd, J = 11.5, 9.1, |
| | | | found, | 2.7 Hz, 1H), 3.93 (s, 3H), 3.65 (t, J = 9.6 Hz, 1H), 3.55- |
| | | | 525.2034 | 3.44 (m, 1H), 2.95 (dd, J = 15.1, 2.6 Hz, 1H), 2.79 |
| | | | | (dd, J = 15.1, 11.0 Hz, 1H), 2.35 (dt, J = 14.8, 6.2 Hz, |
| | | | | 1H), 1.84-1.69 (m, 1H), 1.41 (d, J = 6.6 Hz, 3H) |
| | | | | ¹⁹F NMR (376 MHz, CDCl₃) δ-116.82 |
| 53 | — | — | HRMS-ESI | ¹H NMR (400 MHz, CDCl₃) δ 12.07 (s, 1H), 8.53 (d, |
| | | | (m/z) | J = 8.0 Hz, 1H), 7.96 (d, J = 5.2 Hz, 1H), 7.22 (dd, J = |
| | | | ([M + H]⁺) | 8.5, 5.5 Hz, 2H), 6.98 (t, J = 8.7 Hz, 2H), 6.85 (d, J = |
| | | | calcd for | 5.2 Hz, 1H), 5.13-5.01 (m, 1H), 4.62 (td, J = 8.6, 7.1 |
| | | | C₂₆H₃₄FN₂O₇, | Hz, 1H), 3.92 (s, 3H), 3.81 (ddd, J = 11.5, 9.2, 2.5 Hz, |
| | | | 505.2345; | 1H), 3.56 (t, J = 9.7 Hz, 1H), 3.48 (dt, J = 10.7, 5.5 Hz, |
| | | | found, | 2H), 3.36-3.24 (m, 2H), 3.05 (dd, J = 15.0, 2.4 Hz, |
| | | | 505.2347 | 1H), 2.81 (dd, J = 14.9, 11.2 Hz, 1H), 2.36-2.22 (m, |
| | | | | 1H), 1.85 (hept, J = 6.7 Hz, 1H), 1.76-1.64 (m, 1H), |
| | | | | 1.49 (d, J = 6.5 Hz, 3H), 0.94 (d, J = 6.7 Hz, 3H), 0.93 |
| | | | | (d, J = 6.7 Hz, 3H) |
| | | | | ¹⁹F NMR (376 MHz, CDCl₃) δ-116.99 |
| 54 | — | — | HRMS-ESI | ¹H NMR (400 MHz, CDCl₃) δ 12.03 (s, 1H), 8.51 (d, |
| | | | (m/z) | J = 8.0 Hz, 1H), 7.97 (d, J = 5.2 Hz, 1H), 7.17 (dd, J = |
| | | | ([M + H]⁺) | 8.5, 5.5 Hz, 2H), 6.98 (t, J = 8.7 Hz, 2H), 6.86 (d, J = |
| | | | calcd for | 5.3 Hz, 1H), 5.27 (dq, J = 9.4, 6.3 Hz, 1H), 5.19 (t, J = |
| | | | C₂₆H₃₂FN₂O₈, | 9.4 Hz, 1H), 4.66 (dt, J = 9.4, 7.4 Hz, 1H), 4.00 (ddd, |
| | | | 519.2137; | J = 10.6, 9.3, 3.3 Hz, 1H), 3.93 (s, 3H), 3.70 (t, J = 10.0 |
| | | | found, | Hz, 1H), 3.60-3.51 (m, 1H), 2.89 (dd, J = 15.4, 10.7 |
| | | | 519.214 | Hz, 1H), 2.64 (dd, J = 15.4, 3.3 Hz, 1H), 2.52 (p, J = 7.0 |
| | | | | Hz, 1H), 2.35 (ddd, J = 14.5, 7.3, 4.4 Hz, 1H), 1.81- |
| | | | | 1.64 (m, 1H), 1.30 (d, J = 6.3 Hz, 3H), 1.19 (d, J = 7.0 |
| | | | | Hz, 3H), 1.18 (d, J = 7.0 Hz, 3H) |
| | | | | ¹⁹F NMR (376 MHz, CDCl₃) δ-116.55 |
| 55 | — | — | HRMS-ESI | ¹H NMR (400 MHz, CDCl₃) δ 12.06 (s, 1H), 8.52 (d, |
| | | | (m/z) | J = 7.9 Hz, 1H), 7.96 (d, J = 5.2 Hz, 1H), 7.22 (dd, J = |
| | | | ([M + H]⁺) | 8.5, 5.5 Hz, 2H), 6.97 (t, J = 8.7 Hz, 2H), 6.85 (d, J = |
| | | | calcd for | 5.3 Hz, 1H), 5.90 (ddt, J = 17.2, 10.4, 5.6 Hz, 1H), 5.31 |
| | | | C₂₅H₃₀FN₂O₇, | (dq, J = 17.2, 1.6 Hz, 1H), 5.21 (dq, J = 10.4, 1.3 Hz, |
| | | | 489.2032; | 1H), 5.14-5.01 (m, 1H), 4.62 (td, J = 8.5, 7.0 Hz, 1H), |
| | | | found, | 4.24 (ddt, J = 12.2, 5.5, 1.4 Hz, 1H), 4.10 (ddt, J = 12.2, |
| | | | 489.2032 | 5.7, 1.4 Hz, 1H), 3.92 (s, 3H), 3.84 (ddd, J = 11.6, 9.2, |
| | | | | 2.6 Hz, 1H), 3.55 (t, J = 9.6 Hz, 1H), 3.49-3.41 (m, |
| | | | | 1H), 3.40 (t, J = 9.6 Hz, 1H), 3.08 (dt, J = 14.7, 2.0 Hz, |
| | | | | 1H), 2.88-2.79 (m, 1H), 2.38-2.22 (m, 1H), 1.79- |
| | | | | 1.62 (m, 1H), 1.51 (d, J = 6.6 Hz, 3H) |
| | | | | ¹⁹F NMR (376 MHz, CDCl₃) δ-116.94 |
| 56 | — | — | HRMS-ESI | ¹H NMR (400 MHz, CDCl₃) δ 12.06 (s, 1H), 8.53 (d, |
| | | | (m/z) | J = 8.0 Hz, 1H), 7.97 (d, J = 5.2 Hz, 1H), 7.22 (dd, J = |
| | | | ([M + H]⁺) | 8.5, 5.5 Hz, 2H), 6.98 (t, J = 8.7 Hz, 2H), 6.85 (d, J = |
| | | | calcd for | 5.2 Hz, 1H), 5.12-4.99 (m, 1H), 4.62 (td, J = 8.5, 7.1 |
| | | | C₂₅H₃₂FN₂O₇, | Hz, 1H), 3.93 (s, 3H), 3.81 (ddd, J = 11.5, 9.2, 2.6 Hz, |
| | | | 491.2188; | 1H), 3.67 (dt, J = 8.6, 6.6 Hz, 1H), 3.60-3.48 (m, 2H), |
| | | | found, | 3.48-3.37 (m, 1H), 3.31 (t, J = 9.2 Hz, 1H), 3.11- |
| | | | 491.2183 | 3.00 (m, 1H), 2.82 (dd, J = 14.9, 11.2 Hz, 1H), 2.35- |
| | | | | 2.24 (m, 1H), 1.76-1.62 (m, 1H), 1.60 (h, J = 7.1 Hz, |
| | | | | 2H), 1.50 (d, J = 6.5 Hz, 3H), 0.95 (t, J = 7.4 Hz, 3H) |
| | | | | ¹⁹F NMR (376 MHz, CDCl₃) δ-117.01 |
| 57 | — | — | ESIMS | ¹H NMR (400 MHz, CDCl₃) δ 12.05 (s, 1H), 8.54 (d, |
| | | | m/z 507 | J = 7.9 Hz, 1H), 7.98 (d, J = 5.1 Hz, 1H), 7.38-7.15 (m, |
| | | | ([M + H]⁺) | 7H), 7.03-6.97 (m, 3H), 6.86 (d, J = 5.1 Hz, 1H), 5.29 |
| | | | | (m, 1H), 4.73-4.63 (m, 1H), 4.51 (t, J = 9.2 Hz, 1H), |
| | | | | 4.17-4.05 (m, 1H), 3.93 (s, 3H), 3.64 (t, J = 9.5 Hz, |
| | | | | 1H), 3.56-3.48 (m, 1H), 2.99 (dd, J = 14.9, 2.3 Hz, |
| | | | | 1H), 2.81 (dd, J = 14.9, 11.2 Hz, 1H), 2.39-2.28 (m, |
| | | | | 1H), 1.82-1.69 (m, 1H), 1.42 (d, J = 6.7 Hz, 3H) |
| | | | | ¹³C NMR (101 MHz, CDCl₃) δ 171.49, 168.71, 158.56, |
| | | | | 155.32, 148.70, 140.58, 138.07, 130.35, 129.80, 128.76, |
| | | | | 128.45, 126.32, 121.72, 115.61, 109.48, 82.39, 77.46, |
| | | | | 74.81, 61.69, 56.07, 50.14, 36.44, 34.51, 18.26 |
| 58 | 78- | — | HRMS-ESI | ¹H NMR (500 MHz, CDCl₃) δ 12.06 (d, J = 0.6 Hz, |
| | 83 | | (m/z) | 1H), 8.59 (d, J = 8.1 Hz, 1H), 8.00 (d, J = 5.2 Hz, 1H), |
| | | | ([M + H]⁺) | 7.33-7.27 (m, 2H), 7.02-6.96 (m, 1H), 6.96-6.91 |
| | | | calcd for | (m, 2H), 6.90-6.84 (m, 1H), 5.36-5.26 (m, 1H), 4.66 |
| | | | C₂₇H₃₅N₂O₇, | (dt, J = 10.0, 7.6 Hz, 1H), 4.32 (t, J = 9.2 Hz, 1H), 3.94 |
| | | | 499.2439; | (s, 3H), 3.77 (ddd, J = 11.5, 9.3, 2.3 Hz, 1H), 3.71- |
| | | | found, | 3.64 (m, 2H), 2.47-2.37 (m, 1H), 1.99-1.90 (m, 1H), |
| | | | 499.2438 | 1.89-1.69 (m, 4H), 1.62-1.23 (m, 8H), 1.10-0.99 |
| | | | | (m, 2H) |
| 59 | — | — | HRMS-ESI | ¹H NMR (500 MHz, CDCl₃) δ 12.06 (d, J = 0.5 Hz, |
| | | | (m/z) | 1H), 8.52 (d, J = 8.0 Hz, 1H), 7.97 (d, J = 5.2 Hz, 1H), |
| | | | ([M + H]⁺) | 7.22-7.16 (m, 2H), 6.84 (dd, J = 9.7, 3.1 Hz, 3H), 5.07 |
| | | | calcd for | (s, 1H), 4.70-4.56 (m, 1H), 3.93 (s, 3H), 3.84 (t, J = |
| | | | C₂₇H₃₅N₂O₈, | 9.1 Hz, 1H), 3.78 (s, 3H), 3.53 (dd, J = 9.7, 7.0 Hz, 2H), |
| | | | 515.2388; | 3.44 (dd, J = 9.7, 6.9 Hz, 2H), 3.33 (t, J = 9.2 Hz, 1H), |
| | | | found, | 3.09 (dd, J = 14.8, 2.4 Hz, 1H), 2.76 (dd, J = 14.8, 11.3 |
| | | | 515.2389 | Hz, 1H), 2.28 (dt, J = 12.8, 5.7 Hz, 1H), 1.79-1.62 (m, |
| | | | | 1H), 1.50 (d, J = 6.5 Hz, 3H), 1.18-1.02 (m, 1H), 0.57 |
| | | | | (ddd, J = 7.9, 4.7, 1.8 Hz, 2H), 0.31-0.16 (m, 2H) |
| | | | | ¹³C NMR (126 MHz, CDCl₃) δ 171.48, 168.67, 158.08, |
| | | | | 155.30, 148.67, 140.56, 130.42, 130.38, 129.72, 113.92, |
| | | | | 109.44, 80.33, 78.43, 56.06, 55.25, 50.13, 34.54, 18.06, |
| | | | | 11.06, 3.18, 3.15 |
| 60 | — | — | HRMS-ESI | ¹H NMR (500 MHz, CDCl₃) δ 12.04 (d, J = 0.5 Hz, |
| | | | (m/z) | 1H), 8.55 (d, J = 7.9 Hz, 1H), 7.99 (d, J = 5.2 Hz, 1H), |
| | | | ([M + H]⁺) | 7.35-7.28 (m, 2H), 7.17-7.11 (m, 2H), 7.02-6.96 |
| | | | calcd for | (m, 3H), 6.86 (d, J = 5.1 Hz, 1H), 6.84-6.77 (m, 2H), |
| | | | C₂₉H₃₃N₂O₈, | 5.32-5.23 (m, 1H), 4.72-4.63 (m, 1H), 4.48 (t, J = |
| | | | 537.2231; | 9.1 Hz, 1H), 4.11-4.00 (m, 1H), 3.94 (s, 3H), 3.77 (s, |
| | | | found, | 3H), 3.64 (t, J = 9.9 Hz, 1H), 3.57-3.46 (m, 1H), 2.93 |
| | | | 537.2230 | (dd, J = 15.0, 2.4 Hz, 1H), 2.75 (dd, J = 15.0, 11.3 Hz, |
| | | | | 1H), 2.34 (dt, J = 12.9, 5.8 Hz, 1H), 1.84-1.70 (m, |
| | | | | 1H), 1.41 (d, J = 6.6 Hz, 3H) |
| | | | | ¹³C NMR (126 MHz, CDCl₃) δ 171.49, 168.70, 158.58, |
| | | | | 158.10, 155.33, 148.70, 140.58, 130.35, 130.00, 129.79, |
| | | | | 129.68, 121.69, 115.61, 113.88, 109.48, 56.08, 55.22, |
| | | | | 50.15, 34.54, 18.26 |
| 61 | — | (Thin | HRMS-ESI | ¹H NMR (400 MHz, CDCl₃) δ 12.08 (s, 1H), 8.58 (d, |
| | | Film) | (m/z) | J = 8.1 Hz, 1H), 7.99 (d, J = 5.1 Hz, 1H), 6.87 (d, J = 5.2 |
| | | 3375, | ([M + H]⁺) | Hz, 1H), 5.13 (dq, J = 9.0, 6.5 Hz, 1H), 4.97 (s, 1H), |
| | | 2936, | calcd for | 4.88 (s, 1H), 4.62 (dt, J = 9.9, 7.6 Hz, 1H), 4.07 (d, J = |
| | | 2877, | C₂₁H₃₁N₂O₇, | 11.5 Hz, 1H), 3.94 (s, 3H), 3.91 (d, J = 11.6 Hz, 1H), |
| | | 1744, | 423.2126; | 3.66-3.52 (m, 2H), 3.41 (ddd, J = 11.5, 9.4, 2.6 Hz, |
| | | 1646, | found, | 1H), 3.23 (t, J = 9.3 Hz, 1H), 2.43-2.33 (m, 1H), 1.87- |
| | | 1576, | 423.213 | 1.67 (m, 2H), 1.74 (d, J = 1.2 Hz, 3H), 1.67-1.53 (m, |
| | | 1527, | | 1H), 1.45 (d, J = 6.5 Hz, 3H), 0.99 (t, J = 7.3 Hz, 3H) |
| | | 1433 | | |
| 62 | — | (Thin | HRMS-ESI | ¹H NMR (400 MHz, CDCl₃) δ 12.09 (s, 1H), 8.58 (d, |
| | | Film) | (m/z) | J = 8.1 Hz, 1H), 7.99 (d, J = 5.2 Hz, 1H), 6.87 (d, J = 5.2 |
| | | 3384, | ([M + H]⁺) | Hz, 1H), 5.10 (dq, J = 9.0, 6.5 Hz, 1H), 4.61 (dt, J = 9.9, |
| | | 2952, | calcd for | 7.6 Hz, 1H), 3.94 (s, 3H), 3.69-3.50 (m, 2H), 3.46- |
| | | 2874, | C₂₁H₃₃N₂O₇, | 3.29 (m, 2H), 3.25 (dd, J = 8.4, 6.3 Hz, 1H), 3.14 (t, J = |
| | | 1745, | 425.2282; | 9.3 Hz, 1H), 2.45-2.31 (m, 1H), 1.89-1.75 (m, 2H), |
| | | 1656, | found, | 1.75-1.65 (m, 1H), 1.63-1.51 (m, 1H), 1.42 (d, J = |
| | | 1525 | 425.2285 | 6.5 Hz, 3H), 0.99 (t, J = 7.3 Hz, 3H), 0.91 (d, J = 6.8 |
| | | | | Hz, 3H), 0.90 (d, J = 6.7 Hz, 3H) |
| 63 | — | (Thin | HRMS-ESI | ¹H NMR (400 MHz, CDCl₃) δ 12.06 (s, 1H), 8.60 (d, |
| | | Film) | (m/z) | J = 8.1 Hz, 1H), 8.01 (d, J = 5.1 Hz, 1H), 7.30 (dd, J = |
| | | 3366, | ([M + H]⁺) | 8.7, 7.3 Hz, 2H), 7.01-6.92 (m, 3H), 6.87 (d, J = 5.2 |
| | | 2935, | calcd for | Hz, 1H), 5.36-5.22 (m, 1H), 4.73-4.60 (m, 1H), 4.35 |
| | | 2876, | C₂₃H₂₉N₂O₇, | (t, J = 9.2 Hz, 1H), 3.94 (s, 3H), 3.76-3.54 (m, 3H), |
| | | 1748, | 445.1969; | 2.49-2.36 (m, 1H), 1.85 (dtd, J = 13.6, 10.0, 3.3 Hz, |
| | | 1648, | found, | 1H), 1.73-1.50 (m, 2H), 1.34 (d, J = 6.6 Hz, 3H), 0.97 |
| | | 1527, | 445.1973 | (t, J = 7.3 Hz, 3H) |
| | | 1481, | | |
| | | 1435 | | |
| 64 | — | — | HRMS-ESI | ¹H NMR (400 MHz, CDCl₃) δ 12.05 (s, 1H), 8.59 (d, |
| | | | (m/z) | J = 8.1 Hz, 1H), 8.01 (d, J = 5.2 Hz, 1H), 7.02-6.95 (m, |
| | | | ([M + H]⁺) | 2H), 6.91-6.85 (m, 3H), 5.35-5.21 (m, 1H), 4.66 |
| | | | calcd for | (ddd, J = 9.8, 8.1, 7.2 Hz, 1H), 4.24 (t, J = 9.2 Hz, 1H), |
| | | | C₂₃H₂₈FN₂O₇, | 3.95 (s, 3H), 3.72-3.64 (m, 2H), 3.61 (td, J = 9.2, 4.7 |
| | | | 463.1875; | Hz, 1H), 2.50-2.38 (m, 1H), 1.85 (dtd, J = 13.9, 9.5, |
| | | | found, | 4.1 Hz, 1H), 1.71-1.53 (m, 2H), 1.33 (d, J = 6.6 Hz, |
| | | | 463.1880 | 3H), 0.97 (t, J = 7.3 Hz, 3H) |
| | | | | ¹⁹F NMR (376 MHz, CDCl₃) δ-122.78 |
| 65 | — | (Thin | HRMS-ESI | ¹H NMR (400 MHz, CDCl₃) δ 12.05 (s, 1H), 8.55 (d, |
| | | Film) | (m/z) | J = 8.2 Hz, 1H), 8.00 (d, J = 5.2 Hz, 1H), 6.87 (d, J = 5.2 |
| | | 3368, | ([M + H]⁺) | Hz, 1H), 5.25 (dq, J = 9.2, 6.2 Hz, 1H), 5.05 (t, J = 9.5 |
| | | 2970, | calcd for | Hz, 1H), 4.66 (dt, J = 10.1, 7.7 Hz, 1H), 3.94 (s, 3H), |
| | | 2937, | C₂₁H₃₁N₂O₈, | 3.71-3.58 (m, 2H), 3.51 (ddd, J = 11.7, 9.7, 2.6 Hz, |
| | | 2878, | 439.2075; | 1H), 2.57 (hept, J = 7.0 Hz, 1H), 2.42 (ddd, J = 14.4, |
| | | 1739, | found, | 7.4, 3.3 Hz, 1H), 1.82 (dtd, J = 13.8, 10.7, 2.8 Hz, 1H), |
| | | 1649, | 439.207 | 1.61 (ddq, J = 14.5, 11.5, 7.2 Hz, 1H), 1.38-1.29 (m, |
| | | 1528, | | 1H), 1.26 (d, J = 6.4 Hz, 3H), 1.19 (d, J = 7.0 Hz, 3H), |
| | | 1437 | | 1.18 (d, J = 7.0 Hz, 3H), 0.96 (t, J = 7.4 Hz, 3H) |
| 66 | 140- | — | HRMS-ESI | ¹H NMR (400 MHz, CDCl₃) δ 12.08 (d, J = 0.7 Hz, |
| | 142 | | (m/z) | 1H), 8.73 (d, J = 7.6 Hz, 1H), 7.98 (d, J = 5.2 Hz, 1H), |
| | | | ([M + H]⁺) | 7.40-7.24 (m, 5H), 6.86 (d, J = 5.2 Hz, 1H), 5.30 (p, |
| | | | calcd for | J = 6.6 Hz, 1H), 4.72-4.54 (m, 3H), 3.95-3.84 (m, 4H), |
| | | | C₂₂H₂₇N₂O₇, | 3.78 (dd, J = 11.8, 5.7 Hz, 1H), 3.62 (dd, J = 11.8, 3.5 |
| | | | 431.1813; | Hz, 1H), 3.57-3.45 (m, 2H), 2.53-2.40 (m, 1H), 1.92 |
| | | | found, | (dtd, J = 14.4, 8.0, 2.6 Hz, 1H), 1.37 (d, J = 6.7 Hz, 3H) |
| | | | 431.1814 | |
| 67 | 102- | — | HRMS-ESI | ¹H NMR (400 MHz, CDCl₃) δ 12.08 (d, J = 0.6 Hz, |
| | 105 | | (m/z) | 1H), 8.71 (d, J = 7.7 Hz, 1H), 7.99 (d, J = 5.2 Hz, 1H), |
| | | | ([M + H]⁺) | 6.87 (d, J = 5.2 Hz, 1H), 5.21 (p, J = 6.8 Hz, 1H), 4.67 |
| | | | calcd for | (td, J = 7.9, 5.9 Hz, 1H), 3.94 (s, 3H), 3.86 (ddd, J = |
| | | | C₁₉H₂₉N₂O₇, | 10.9, 6.9, 2.6 Hz, 1H), 3.69 (d, J = 5.1 Hz, 2H), 3.56 |
| | | | 397.1969; | (ddd, J = 10.7, 8.3, 2.1 Hz, 1H), 3.41 (dt, J = 7.1, 5.1 |
| | | | found, | Hz, 1H), 3.31 (dd, J = 8.8, 6.2 Hz, 1H), 3.21 (dd, J = |
| | | | 397.1973 | 8.8, 6.9 Hz, 1H), 2.46 (dddd, J = .14.8, 6.9, 5.9, 2.2 Hz, |
| | | | | 1H), 1.97-1.76 (m, 2H), 1.42 (d, J = 6.6 Hz, 3H), 0.91 |
| | | | | (d, J = 6.7 Hz, 3H), 0.89 (d, J = 6.7 Hz, 3H) |
| 68 | 124- | — | HRMS-ESI | ¹H NMR (400 MHz, CDCl₃) δ 12.07 (s, 1H), 8.73 (d, |
| | 126 | | (m/z) | J = 7.6 Hz, 1H), 7.99 (d, J = 5.2 Hz, 1H), 6.87 (d, J = 5.2 |
| | | | ([M + H]⁺) | Hz, 1H), 5.23 (p, J = 6.6 Hz, 1H), 4.68 (td, J = 7.7, 5.6 |
| | | | calcd for | Hz, 1H), 3.98-3.86 (m, 4H), 3.75 (dd, J = 11.8, 5.4 |
| | | | C₁₉H₂₆F₃N₂O₇, | Hz, 1H), 3.69-3.46 (m, 4H), 3.39 (ddd, J = 6.4, 5.4, |
| | | | 451.1687; | 3.5 Hz, 1H), 2.48 (dddd, J = 14.7, 7.7, 5.7, 2.4 Hz, 1H), |
| | | | found, | 2.29-2.12 (m, 2H), 1.99-1.78 (m, 3H), 1.41 (d, J = |
| | | | 451.1693 | 6.7 Hz, 3H) |
| 69 | 144- | — | HRMS-ESI | ¹H NMR (400 MHz, CDCl₃) δ 12.06 (d, J = 0.7 Hz, |
| | 146 | | (m/z) | 1H), 8.72 (d, J = 7.7 Hz, 1H), 8.00 (d, J = 5.2 Hz, 1H), |
| | | | ([M + H]⁺) | 7.34-7.24 (m, 2H), 7.04-6.89 (m, 3H), 6.87 (d, J = |
| | | | calcd for | 5.2 Hz, 1H), 5.42 (p, J = 6.6 Hz, 1H), 4.72 (td, J = 8.0, |
| | | | C₂₁H₂₅N₂O₇, | 6.0 Hz, 1H), 4.38 (td, J = 6.1, 3.8 Hz, 1H), 4.00-3.92 |
| | | | 417.1656; | (m, 4H), 3.89 (dd, J = 12.1, 5.8 Hz, 1H), 3.81 (dd, J = |
| | | | found, | 12.1, 3.8 Hz, 1H), 3.62 (ddd, J = 10.8, 8.5, 2.1 Hz, 1H), |
| | | | 417.1662 | 2.55-2.42 (m, 1H), 2.02-1.89 (m, 1H), 1.45 (d, J = |
| | | | | 6.6 Hz, 3H) |
| 70 | 153- | — | HRMS-ESI | ¹H NMR (400 MHz, CDCl₃) δ 12.05 (d, J = 0.7 Hz, |
| | 155 | | (m/z) | 1H), 8.66 (d, J = 7.7 Hz, 1H), 7.99 (d, J = 5.2 Hz, 1H), |
| | | | ([M + H]⁺) | 6.88 (d, J = 5.2 Hz, 1H), 5.29 (qd, J = 6.7, 5.1 Hz, 1H), |
| | | | calcd for | 4.80 (td, J = 5.2, 3.2 Hz, 1H), 4.70 (ddd, J = 8.6, 7.7, 5.8 |
| | | | C₁₉H₂₇N₂O₈, | Hz, 1H), 4.00 (ddd, J = 10.9, 6.5, 2.6 Hz, 1H), 3.94 (s, |
| | | | 411.1762; | 3H), 3.79 (dd, J = 12.1, 5.2 Hz, 1H), 3.69 (dd, J = 12.0, |
| | | | found, | 3.3 Hz, 1H), 3.57 (ddd, J = 10.9, 8.7, 2.1 Hz, 1H), 2.62 |
| | | | 411.1766 | (p, J = 7.0 Hz, 1H), 2.46 (dtd, J = 14.5, 6.3, 2.1 Hz, 1H), |
| | | | | 1.93 (dtd, J = 14.4, 8.7, 2.6 Hz, 1H), 1.40 (d, J = 6.7 Hz, |
| | | | | 3H), 1.21 (d, J = 7.0 Hz, 3H), 1.18 (d, J = 7.0 Hz, 3H) |
| 71 | — | — | ESIMS | ¹H NMR (400 MHz, CDCl₃) δ 8.57 (d, J = 7.4 Hz, 1H), |
| | | | m/z 529 | 8.31 (d, J = 5.4 Hz, 1H), 7.34-7.15 (m, 5H), 6.98 (d, |
| | | | ([M + H]⁺) | J = 5.5 Hz, 1H), 5.14-5.02 (m, 1H), 4.65-4.57 (m, 1H), |
| | | | | 3.88 (s, 3H), 3.86 (m, 1H), 3.57-3.44 (m, 3H), 3.37- |
| | | | | 3.25 (m, 2H), 3.08 (dd, J = 14.8, 2.1 Hz, 1H), 2.83 (dd, |
| | | | | J = 14.9, 11.2 Hz, 1H), 2.38 (s, 3H), 2.27 (dt, J = 12.4, |
| | | | | 5.6 Hz, 1H), 1.84 (dp, J = 13.2, 6.6 Hz, 1H), 1.63 (dtd, |
| | | | | J = 14.1, 9.8, 2.3 Hz, 1H), 1.46 (d, J = 6.7 Hz, 3H), 0.93 |
| | | | | (d, J = 6.8 Hz, 6H) |
| | | | | ¹³C NMR (101 MHz, CDCl₃) δ 172.25, 169.06, 162.65, |
| | | | | 159.99, 146.95, 141.62, 138.92, 137.63, 128.99, 128.65, |
| | | | | 126.41, 109.99, 82.84, 80.51, 80.41, 75.29, 61.31, |
| | | | | 56.48, 50.41, 36.24, 34.95, 29.36, 20.94, 19.62, 18.28 |
| 72 | — | — | ESIMS | ¹H NMR (400 MHz, CDCl₃) δ 8.35 (d, J = 8.0 Hz, 1H), |
| | | | m/z 559 | 8.26 (d, J = 5.4 Hz, 1H), 7.33-7.15 (m, 5H), 6.93 (d, |
| | | | ([M + H]⁺) | J = 5.4 Hz, 1H), 5.71 (m, 2H), 5.14-5.03 (m, 1H), 4.69- |
| | | | | 4.59 (m, 1H), 3.90 (s, 3H), 3.86 (m, 1H), 3.60-3.45 |
| | | | | (m, 3H), 3.37-3.26 (m, 2H), 3.08 (dd, J = 14.8, 2.1 Hz, |
| | | | | 1H), 2.84 (dd, J = 14.8, 11.3 Hz, 1H), 2.30 (dt, J = 12.2, |
| | | | | 5.4 Hz, 1H), 2.05 (s, 3H), 1.91-1.79 (m, 1H), 1.64 |
| | | | | (ddt, J = 11.8, 9.9, 5.0 Hz, 1H), 1.47 (d, J = 6.5 Hz, 3H), |
| | | | | 0.94 (d, J = 6.8 Hz, 6H) |
| | | | | ¹³C NMR (101 MHz, CDCl₃) δ 172.38, 170.42, 163.20, |
| | | | | 160.39, 146.02, 143.99, 142.74, 138.93, 128.99, 128.65, |
| | | | | 126.40, 109.77, 89.68, 82.76, 80.52, 80.42, 75.25, |
| | | | | 61.27, 56.39, 50.66, 36.20, 34.85, 29.37, 21.08, 19.63, |
| | | | | 18.30 |
| 73 | — | — | HRMS-ESI | ¹H NMR (400 MHz, CDCl₃) δ 8.40 (d, J = 7.8 Hz, 1H), |
| | | | (m/z) | 8.27 (d, J = 5.3 Hz, 1H), 7.31 (dd, J = 8.5, 7.3 Hz, 2H), |
| | | | ([M + H]⁺) | 7.22-7.12 (m, 2H), 7.02-6.91 (m, 6H), 5.73 (s, 2H), |
| | | | calcd for | 5.39-5.16 (m, 1H), 4.79-4.64 (m, 1H), 4.48 (t, J = |
| | | | C₃₁H₃₄FN₂O₉, | 9.1 Hz, 1H), 4.05 (ddd, J = 11.7, 9.1, 2.8 Hz, 1H), 3.90 |
| | | | 597.2243; | (s, 3H), 3.65 (t, J = 9.7 Hz, 1H), 3.50 (ddd, J = 10.0, |
| | | | found, | 5.6, 2.3 Hz, 1H), 2.98-2.87 (m, 1H), 2.80 (dd, J = |
| | | | 597.2245 | 15.1, 11.0 Hz, 1H), 2.41-2.29 (m, 1H), 2.06 (s, 3H), |
| | | | | 1.79-1.65 (m, 1H), 1.39 (d, J = 6.5 Hz, 3H) |
| | | | | ¹⁹F NMR (376 MHz, CDCl₃) δ-116.91 |
| 74 | — | — | HRMS-ESI | ¹H NMR (400 MHz, CDCl₃) δ 8.36 (d, J = 7.8 Hz, 1H), |
| | | | (m/z) | 8.26 (d, J = 5.4 Hz, 1H), 7.17 (dd, J = 8.6, 5.4 Hz, 2H), |
| | | | ([M + H]⁺) | 7.01-6.91 (m, 3H), 5.76-5.68 (m, 2H), 5.33-5.22 |
| | | | calcd for | (m, 1H), 5.18 (t, J = 9.4 Hz, 1H), 4.69 (dt, J = 9.5, 7.4 |
| | | | C₂₉H₃₆FN₂O₁₀, | Hz, 1H), 3.99 (ddd, J = 10.7, 9.3, 3.2 Hz, 1H), 3.90 (s, |
| | | | 591.2349; | 3H), 3.70 (t, J = 10.0 Hz, 1H), 3.53 (ddd, J = 9.7, 4.4, |
| | | | found, | 2.2 Hz, 1H), 2.90 (dd, J = 15.4, 10.7 Hz, 1H), 2.63 (dd, |
| | | | 591.2355 | J = 15.7, 3.3 Hz, 1H), 2.51 (hept, J = 7.0 Hz, 1H), 2.37 |
| | | | | (ddd, J = 14.5, 7.3, 4.3 Hz, 1H), 2.06 (s, 3H), 1.69 |
| | | | | (dddd, J = 14.3, 10.8, 9.5, 2.3 Hz, 1H), 1.29 (d, J = 6.3 |
| | | | | Hz, 3H), 1.19 (d, J = 7.0 Hz, 3H), 1.18 (d, J = 7.0 Hz, |
| | | | | 3H) |
| | | | | ¹⁹F NMR (376 MHz, CDCl₃) δ-116.65 |
| 75 | — | — | HRMS-ESI | ¹H NMR (400 MHz, CDCl₃) δ 8.61 (d, J = 8.0 Hz, 1H), |
| | | | (m/z) | 8.32 (d, J = 5.5 Hz, 1H), 7.34-7.28 (m, 2H), 7.20- |
| | | | ([M + H]⁺) | 7.13 (m, 2H), 7.02-6.97 (m, 4H), 6.94 (t, J = 8.7 Hz, |
| | | | calcd for | 2H), 5.39-5.16 (m, 1H), 4.68 (td, J = 8.4, 7.0 Hz, 1H), |
| | | | C₃₀H₃₂FN₂O₈, | 4.47 (t, J = 9.2 Hz, 1H), 4.04 (ddd, J = 11.5, 9.1, 2.7 Hz, |
| | | | 567.2137; | 1H), 3.89 (s, 3H), 3.62 (t, J = 9.6 Hz, 1H), 3.54-3.37 |
| | | | found, | (m, 1H), 2.93 (dd, J = 15.2, 2.6 Hz, 1H), 2.79 (dd, J = |
| | | | 567.2141 | 15.1, 11.0 Hz, 1H), 2.39 (s, 3H), 2.36-2.28 (m, 1H), |
| | | | | 1.79-1.55 (m, 1H), 1.38 (d, J = 6.6 Hz, 3H) |
| | | | | ¹⁹F NMR (376 MHz, CDCl₃) δ-116.91 |
| 76 | — | — | HRMS-ESI | ¹H NMR (400 MHz, CDCl₃) δ 8.57 (d, J = 8.1 Hz, 1H), |
| | | | (m/z) | 8.31 (d, J = 5.5 Hz, 1H), 7.16 (dd, J = 8.6, 5.5 Hz, 2H), |
| | | | ([M + H]⁺) | 7.06-6.92 (m, 3H), 5.25 (dq, J = 9.4, 6.3 Hz, 1H), 5.16 |
| | | | calcd for | (t, J = 9.4 Hz, 1H), 4.67 (ddd, J = 9.5, 8.1, 7.2 Hz, 1H), |
| | | | C₂₈H₃₄FN₂O₉, | 3.98 (ddd, J = 10.6, 9.3, 3.3 Hz, 1H), 3.89 (s, 3H), 3.66 |
| | | | 561.2243; | (t, J = 10.0 Hz, 1H), 3.51 (ddd, J = 9.5, 4.6, 2.2 Hz, 1H), |
| | | | found, | 2.88 (dd, J = 15.4, 10.6 Hz, 1H), 2.62 (dd, J = 15.4, 3.3 |
| | | | 561.224 | Hz, 1H), 2.51 (hept, J = 7.0 Hz, 1H), 2.39 (s, 3H), 2.36- |
| | | | | 2.29 (m, 1H), 1.74-1.60 (m, 1H), 1.28 (d, J = 6.3 Hz, |
| | | | | 3H), 1.18 (d, J = 7.0 Hz, 3H), 1.17 (d, J = 7.0 Hz, 3H) |
| | | | | ¹⁹F NMR (376 MHz, CDCl₃) δ-116.63 |
| 77 | — | — | HRMS-ESI | ¹H NMR (400 MHz, CDCl₃) δ 8.58 (d, J = 8.2 Hz, 1H), |
| | | | (m/z) | 8.30 (d, J = 5.5 Hz, 1H), 7.21 (dd, J = 8.6, 5.5 Hz, 2H), |
| | | | ([M + H]⁺) | 7.02-6.93 (m, 3H), 5.15-4.98 (m, 1H), 4.72-4.50 |
| | | | calcd for | (m, 1H), 3.88 (s, 3H), 3.79 (ddd, J = 11.5, 9.2, 2.4 Hz, |
| | | | C₂₈H₃₆FN₂O₈, | 1H), 3.57-3.46 (m, 2H), 3.46-3.40 (m, 1H), 3.32 (dd, |
| | | | 547.245; | J = 8.4, 6.4 Hz, 1H), 3.27 (t, J = 9.5 Hz, 1H), 3.08- |
| | | | found, | 2.99 (m, 1H), 2.81 (dd, J = 14.9, 11.2 Hz, 1H), 2.38 (s, |
| | | | 547.2451 | 3H), 2.34-2.22 (m, 1H), 1.92-1.77 (m, 1H), 1.69- |
| | | | | 1.57 (m, 1H), 1.46 (d, J = 6.5 Hz, 3H), 0.93 (d, J = 6.7 |
| | | | | Hz, 3H), 0.93 (d, J = 6.7 Hz, 3H) |
| | | | | ¹⁹F NMR (376 MHz, CDCl₃) δ-117.08 |
| 78 | — | — | HRMS-ESI | ¹H NMR (400 MHz, CDCl₃) δ 8.36 (d, J = 7.9 Hz, 1H), |
| | | | (m/z) | 8.26 (d, J = 5.4 Hz, 1H), 7.22 (dd, J = 8.5, 5.5 Hz, 2H), |
| | | | ([M + H]⁺) | 6.97 (t, J = 8.8 Hz, 2H), 6.94 (d, J = 5.4 Hz, 1H), 5.90 |
| | | | calcd for | (ddt, J = 17.2, 10.4, 5.6 Hz, 1H), 5.72 (s, 2H), 5.31 (dq, |
| | | | C₂₈H₃₄FN₂O₉, | J = 16.9, 1.5 Hz, 1H), 5.21 (dq, J = 10.5, 2.7 Hz, 1H), |
| | | | 561.2243; | 5.08 (dq, J = 12.7, 5.9, 5.3 Hz, 1H), 4.71-4.57 (m, |
| | | | found, | 1H), 4.24 (ddt, J = 12.1, 5.5, 1.4 Hz, 1H), 4.10 (ddt, J = |
| | | | 561.2239 | 12.1, 5.6, 1.4 Hz, 1H), 3.90 (s, 3H), 3.83 (ddd, J = 11.5, |
| | | | | 9.2, 2.6 Hz, 1H), 3.55 (t, J = 9.7 Hz, 1H), 3.48-3.43 |
| | | | | (m, 1H), 3.39 (t, J = 9.2 Hz, 1H), 3.06 (d, J = 14.9 Hz, |
| | | | | 1H), 2.83 (dd, J = 15.0, 11.2 Hz, 1H), 2.37-2.24 (m, |
| | | | | 1H), 2.05 (s, 3H), 1.66 (dtd, J = 14.4, 9.9, 2.2 Hz, 1H), |
| | | | | 1.49 (d, J = 6.6 Hz, 3H) |
| | | | | ¹⁹F NMR (376 MHz, CDCl₃) δ-117.05 |
| 79 | — | — | HRMS-ESI | ¹H NMR (400 MHz, CDCl₃) δ 8.36 (d, J = 7.9 Hz, 1H), |
| | | | (m/z) | 8.26 (d, J = 5.3 Hz, 1H), 7.22 (dd, J = 8.5, 5.5 Hz, 2H), |
| | | | ([M + H]⁺) | 6.97 (t, J = 8.7 Hz, 2H), 6.94 (d, J = 5.5 Hz, 1H), 5.72 |
| | | | calcd for | (s, 2H), 5.13-4.95 (m, 1H), 4.71-4.56 (m, 1H), 3.90 |
| | | | C₂₈H₃₆FN₂O₉, | (s, 3H), 3.80 (ddd, J = 11.5, 9.2, 2.6 Hz, 1H), 3.67 (dt, |
| | | | 563.2399; | J = 8.6, 6.5 Hz, 1H), 3.60-3.47 (m, 2H), 3.47-3.40 (m, |
| | | | found, | 1H), 3.30 (t, J = 9.2 Hz, 1H), 3.05 (dt, J = 14.8, 2.0 Hz, |
| | | | 563.2404 | 1H), 2.83 (dd, J = 15.0, 11.2 Hz, 1H), 2.38-2.24 (m, |
| | | | | 1H), 2.05 (s, 3H), 1.70-1.63 (m, 1H), 1.60 (h, J = 7.1 |
| | | | | Hz, 2H), 1.48 (d, J = 6.5 Hz, 3H), 0.95 (t, J = 7.4 Hz, |
| | | | | 3H) |
| | | | | ¹⁹F NMR (376 MHz, CDCl₃) δ-117.11 |
| 80 | — | — | HRMS-ESI | ¹H NMR (400 MHz, CDCl₃) δ 8.57 (d, J = 8.1 Hz, 1H), |
| | | | (m/z) | 8.31 (d, J = 5.5 Hz, 1H), 7.21 (dd, J = 8.5, 5.5 Hz, 2H), |
| | | | ([M + H]⁺) | 7.01-6.94 (m, 3H), 5.90 (ddt, J = 17.2, 10.3, 5.6 Hz, |
| | | | calcd for | 1H), 5.31 (dq, J = 17.2, 1.6 Hz, 1H), 5.21 (dq, J = 10.4, |
| | | | C₂₇H₃₂FN₂O₈, | 1.3 Hz, 1H), 5.07 (dt, J = 14.2, 6.4 Hz, 1H), 4.62 (td, |
| | | | 531.2137; | J = 8.5, 7.0 Hz, 1H), 4.30-4.19 (m, 1H), 4.09 (ddt, J = |
| | | | found, | 12.1, 5.6, 1.4 Hz, 1H), 3.89 (s, 3H), 3.82 (ddd, J = 11.5, |
| | | | 531.2136 | 9.2, 2.6 Hz, 1H), 3.52 (t, J = 9.6 Hz, 1H), 3.46-3.36 |
| | | | | (m, 1H), 3.38 (t, J = 9.2 Hz, 1H), 3.06 (d, J = 14.4 Hz, |
| | | | | 1H), 2.81 (dd, J = 14.9, 11.2 Hz, 1H), 2.38 (s, 3H), 2.28 |
| | | | | (dt, J = 13.0, 6.0 Hz, 1H), 1.69-1.58 (m, 1H), 1.48 (d, |
| | | | | J = 6.5 Hz, 3H) |
| | | | | ¹⁹F NMR (376 MHz, CDCl₃) δ-117.04 |
| 81 | — | — | HRMS-ESI | ¹H NMR (400 MHz, CDCl₃) δ 8.57 (d, J = 8.1 Hz, 1H), |
| | | | (m/z) | 8.31 (d, J = 5.4 Hz, 1H), 7.21 (dd, J = 8.6, 5.5 Hz, 2H), |
| | | | ([M + H]⁺) | 7.03-6.90 (m, 3H), 5.19-4.95 (m, 1H), 4.62 (td, J = |
| | | | calcd for | 8.7, 7.1 Hz, 1H), 3.89 (s, 3H), 3.79 (ddd, J = 11.5, 9.2, |
| | | | C₂₇H₃₄FN₂O₈, | 2.6 Hz, 1H), 3.66 (dt, J = 8.6, 6.6 Hz, 1H), 3.56-3.47 |
| | | | 533.2294; | (m, 2H), 3.46-3.39 (m, 1H), 3.29 (t, J = 9.2 Hz, 1H), |
| | | | found, | 3.09-3.00 (m, 1H), 2.81 (dd, J = 14.9, 11.2 Hz, 1H), |
| | | | 533.2295 | 2.38 (s, 3H), 2.34-2.22 (m, 1H), 1.71-1.52 (m, 3H), |
| | | | | 1.47 (d, J = 6.5 Hz, 3H), 0.94 (t, J = 7.4 Hz, 3H) |
| | | | | ¹⁹F NMR (376 MHz, CDCl₃) δ-117.10 |
| 82 | — | — | ESIMS | ¹H NMR (400 MHz, CDCl₃) δ 8.39 (d, J = 7.8 Hz, 1H), |
| | | | m/z 579 | 8.27 (d, J = 5.3 Hz, 1H), 7.35-7.15 (m, 7H), 7.00 (d, |
| | | | ([M + H]⁺) | J = 7.5 Hz, 3H), 6.94 (d, J = 5.4 Hz, 1H), 5.72 (d, J = 3.0 |
| | | | | Hz, 2H), 5.29 (m, 1H), 4.70 (q, J = 8.2, 7.8 Hz, 1H), |
| | | | | 4.49 (t, J = 9.2 Hz, 1H), 4.16-4.06 (m, 1H), 3.90 (s, |
| | | | | 3H), 3.65 (t, J = 9.5 Hz, 1H), 3.57-3.49 (m, 1H), 2.98 |
| | | | | (dd, J = 15.1, 2.4 Hz, 1H), 2.83 (dd, J = 14.9, 11.2 Hz, |
| | | | | 1H), 2.35 (dt, J = 12.7, 5.7 Hz, 1H), 2.06 (s, 3H), 1.76- |
| | | | | 1.69 (m, 1H), 1.40 (d, J = 6.5 Hz, 3H) |
| | | | | ¹³C NMR (101 MHz, CDCl₃) δ 172.16, 170.25, 163.02, |
| | | | | 160.20, 158.61, 145.82, 143.84, 142.44, 138.15, 129.79, |
| | | | | 128.75, 128.43, 126.28, 121.68, 115.62, 109.61, 89.46, |
| | | | | 82.26, 77.52, 74.48, 61.62, 56.19, 50.48, 36.31, 34.64, |
| | | | | 20.87, 18.26 |
| 83 | — | — | ESIMS | ¹H NMR (400 MHz, CDCl₃) δ 8.60 (d, J = 7.7 Hz, 1H), |
| | | | m/z 549 | 8.32 (d, J = 5.4 Hz, 1H), 7.35-7.13 (m, 7H), 7.04- |
| | | | ([M + H]⁺) | 6.94 (m, 4H), 5.27 (m, 1H), 4.67 (q, J = 8.3 Hz, 1H), |
| | | | | 4.48 (t, J = 9.2 Hz, 1H), 4.14-4.06 (m, 1H), 3.89 (s, |
| | | | | 3H), 3.61 (t, J = 9.5 Hz, 1H), 3.56-3.44 (m, 1H), 2.97 |
| | | | | (dd, J = 15.0, 2.2 Hz, 1H), 2.81 (dd, J = 14.9, 11.2 Hz, |
| | | | | 1H), 2.39 (s, 3H), 2.36-2.27 (m, 1H), 1.75-1.64 (m, |
| | | | | 1H), 1.39 (d, J = 6.5 Hz, 3H) |
| | | | | ¹³C NMR (101 MHz, CDCl₃) δ 172.00, 168.88, 162.46, |
| | | | | 159.40, 158.60, 146.75, 141.36, 138.15, 137.45, 129.78, |
| | | | | 128.76, 128.43, 126.28, 121.67, 115.63, 109.81, 82.35, |
| | | | | 77.54, 74.53, 61.77, 56.29, 50.22, 36.37, 34.77, 20.74, |
| | | | | 18.25 |
| 84 | — | — | ESIMS | ¹H NMR (400 MHz, CDCl₃) δ 8.45 (d, J = 7.9 Hz, 1H), |
| | | | m/z 607 | 8.27 (d, J = 5.3 Hz, 1H), 7.36-7.13 (m, 7H), 7.04- |
| | | | ([M + H]⁺) | 6.95 (m, 3H), 6.93 (d, J = 5.5 Hz, 1H), 5.80-5.71 (m, |
| | | | | 2H), 5.34-5.24 (m, 1H), 4.70 (q, J = 8.2 Hz, 1H), 4.49 |
| | | | | (t, J = 9.2 Hz, 1H), 4.16-4.06 (m, 1H), 3.88 (s, 3H), |
| | | | | 3.65 (t, J = 9.7 Hz, 1H), 3.53 (d, J = 5.3 Hz, 1H), 2.98 |
| | | | | (dd, J = 15.1, 2.4 Hz, 1H), 2.82 (dd, J = 14.9, 11.2 Hz, |
| | | | | 1H), 2.53 (hept, J = 6.9 Hz, 1H), 2.35 (dt, J = 12.7, 5.6 |
| | | | | Hz, 1H), 1.77-1.66 (m, 1H), 1.40 (d, J = 6.7 Hz, 3H), |
| | | | | 1.13 (d, J = 7.0 Hz, 6H) |
| | | | | ¹³C NMR (101 MHz, CDCl₃) δ 176.22, 172.14, 162.97, |
| | | | | 160.19, 158.61, 145.68, 144.05, 142.10, 138.15, 129.79, |
| | | | | 128.75, 128.43, 126.27, 121.67, 115.62, 109.54, 89.80, |
| | | | | 82.26, 77.53, 74.50, 61.75, 56.13, 50.47, 36.32, 34.66, |
| | | | | 33.85, 18.67, 18.26 |
| 85 | 67- | — | HRMS-ESI | ¹H NMR (400 MHz, CDCl₃) δ 8.43 (d, J = 7.9 Hz, 1H), |
| | 72 | | (m/z) | 8.29 (d, J = 5.4 Hz, 1H), 7.34-7.24 (m, 2H), 7.02- |
| | | | ([M + H]⁺) | 6.90 (m, 4H), 5.75 (app d, J = 1.1 Hz, 2H), 5.29 (dq, J = |
| | | | calcd for | 9.6, 6.6 Hz, 1H), 4.68 (dt, J = 10.1, 7.6 Hz, 1H), 4.31 (t, |
| | | | C₃₀H₃₉N₂O₉, | J = 9.2 Hz, 1H), 3.91 (s, 3H), 3.76 (ddd, J = 11.5, 9.3, |
| | | | 571.2650; | 2.2 Hz, 1H), 3.70-3.62 (m, 2H), 2.50-2.38 (m, 1H), |
| | | | found, | 2.07 (s, 3H), 2.00-1.89 (m, 1H), 1.88-1.71 (m, 4H), |
| | | | 571.2644 | 1.60-1.28 (m, 8H), 1.11-0.98 (m, 2H) |
| 86 | — | — | HRMS-ESI | ¹H NMR (500 MHz, CDCl₃) δ 8.35 (d, J = 7.9 Hz, 1H), |
| | | | (m/z) | 8.26 (d, J = 5.4 Hz, 1H), 7.18 (d, J = 8.6 Hz, 2H), 6.93 |
| | | | ([M + H]⁺) | (d, J = 5.4 Hz, 1H), 6.88-6.77 (m, 2H), 5.72 (d, J = 1.3 |
| | | | calcd for | Hz, 2H), 5.08 (s, 1H), 4.70-4.57 (m, 1H), 3.90 (s, 3H), |
| | | | C₃₀H₃₉N₂O₁₀, | 3.87-3.81 (m, 1H), 3.78 (s, 3H), 3.53 (dd, J = 9.7, 7.0 |
| | | | 587.2599; | Hz, 2H), 3.43 (dd, J = 9.7, 6.9 Hz, 2H), 3.31 (t, J = 9.2 |
| | | | found, | Hz, 1H), 3.08 (dd, J = 14.8, 2.3 Hz, 1H), 2.77 (dd, J = |
| | | | 587.2600 | 14.8, 11.3 Hz, 1H), 2.29 (dt, J = 12.7, 5.7 Hz, 1H), 2.05 |
| | | | | (s, 3H), 1.72-1.60 (m, 1H), 1.48 (d, J = 6.5 Hz, 3H), |
| | | | | 1.08 (tt, J = 7.9, 4.8 Hz, 1H), 0.62-0.53 (m, 2H), 0.30- |
| | | | | 0.19 (m, 2H) |
| | | | | ¹³C NMR (126 MHz, CDCl₃) δ 170.25, 162.98, 160.18, |
| | | | | 158.04, 145.83, 143.78, 142.50, 130.50, 129.70, 113.90, |
| | | | | 109.55, 89.47, 80.37, 78.40, 56.17, 55.25, 50.45, 34.66, |
| | | | | 29.28, 20.87, 18.03, 11.06, 3.18, 3.14 |
| 87 | — | — | HRMS-ESI | ¹H NMR (500 MHz, CDCl₃) δ 8.39 (d, J = 7.9 Hz, 1H), |
| | | | (m/z) | 8.27 (d, J = 5.4 Hz, 1H), 7.31 (dd, J = 8.6, 7.5 Hz, 2H), |
| | | | ([M + H]⁺) | 7.13 (d, J = 8.7 Hz, 2H), 7.04-6.96 (m, 3H), 6.94 (d, |
| | | | calcd for | J = 5.4 Hz, 1H), 6.86-6.75 (m, 2H), 5.73 (d, J = 0.9 Hz, |
| | | | C₃₂H₃₇N₂O₁₀, | 2H), 5.36-5.21 (m, 1H), 4.76-4.64 (m, 1H), 4.47 (t, |
| | | | 609.2443; | J = 9.2 Hz, 1H), 4.12-3.99 (m, 1H), 3.91 (s, 3H), 3.77 |
| | | | found, | (s, 3H), 3.64 (t, J = 9.6 Hz, 1H), 3.52 (s, 1H), 2.91 (dd, |
| | | | 609.2446 | J = 15.0, 2.4 Hz, 1H), 2.76 (dd, J = 15.0, 11.3 Hz, 1H), |
| | | | | 2.35 (dt, J = 12.7, 5.8 Hz, 1H), 2.06 (s, 3H), 1.81-1.66 |
| | | | | (m, 1H), 1.39 (d, J = 6.6 Hz, 3H) |
| | | | | ¹³C NMR (126 MHz, CDCl₃) δ 172.16, 170.27, 163.01, |
| | | | | 160.21, 158.62, 158.06, 145.82, 143.85, 142.43, 130.09, |
| | | | | 129.77, 129.66, 121.64, 115.62, 113.86, 109.59, 89.48, |
| | | | | 56.19, 55.22, 53.78, 50.48, 34.66, 31.74, 29.28, 20.88, |
| | | | | 18.25 |
| 88 | — | (Thin | HRMS-ESI | ¹H NMR (500 MHz, CDCl₃) δ 8.40 (d, J = 8.1 Hz, 1H), |
| | | Film) | (m/z) | 8.28 (d, J = 5.3 Hz, 1H), 6.94 (d, J = 5.4 Hz, 1H), 5.80- |
| | | 3377, | ([M + H]⁺) | 5.62 (m, 2H), 5.16-5.05 (m, 1H), 4.97 (dq, J = 2.2, 1.1 |
| | | 2970, | calcd for | Hz, 1H), 4.92-4.84 (m, 1H), 4.65 (dt, J = 9.9, 7.6 Hz, |
| | | 2937, | C₂₄H₃₅N₂O₉, | 1H), 4.07 (d, J = 11.5 Hz, 1H), 3.91 (s, 3H), 3.91 (d, J = |
| | | 2877, | 495.2337; | 11.5 Hz, 1H), 3.62-3.52 (m, 2H), 3.40 (ddd, J = 11.6, |
| | | 1753, | found, | 9.3, 2.6 Hz, 1H), 3.22 (t, J = 9.3 Hz, 1H), 2.45-2.36 |
| | | 1676, | 495.2341 | (m, 1H), 2.07 (s, 3H), 1.74 (s, 3H), 1.81-1.67 (m, 2H), |
| | | 1504 | | 1.60 (ddt, J = 14.3, 11.3, 7.2 Hz, 1H), 1.43 (d, J = 6.6 |
| | | | | Hz, 3H), 0.99 (t, J = 7.3 Hz, 3H) |
| 89 | — | (Thin | HRMS-ESI | ¹H NMR (500 MHz, CDCl₃) δ 8.40 (d, J = 8.0 Hz, 1H), |
| | | Film) | (m/z) | 8.28 (d, J = 5.3 Hz, 1H), 6.94 (d, J = 5.4 Hz, 1H), 5.80- |
| | | 3376, | ([M + H]⁺) | 5.68 (m, 2H), 5.17-5.00 (m, 1H), 4.64 (dt, J = 9.9, 7.6 |
| | | 2960, | calcd for | Hz, 1H), 3.91 (s, 3H), 3.65-3.50 (m, 2H), 3.41 (dd, J = |
| | | 2876, | C₂₄H₃₇N₂O₉, | 8.3, 6.6 Hz, 1H), 3.36 (ddd, J = 11.5, 9.2, 2.5 Hz, 1H), |
| | | 1752, | 497.2494; | 1.64-1.48 (m, 1H), 3.25 (dd, J = 8.3, 6.3 Hz, 1H), 3.13 |
| | | 1676, | found, | (t, J = 9.3 Hz, 1H), 2.45-2.33 (m, 1H), 2.07 (s, 3H), |
| | | 1504 | 497.2497 | 1.90-1.77 (m, 1H), 1.77-1.67 (m, 2H), 1.41 (d, J = |
| | | | | 6.6 Hz, 3H), 0.99 (t, J = 7.3 Hz, 3H), 0.91 (d, J = 6.7 |
| | | | | Hz, 3H), 0.90 (d, J = 6.6 Hz, 3H) |
| 90 | — | (Thin | HRMS-ESI | ¹H NMR (500 MHz, CDCl₃) δ 8.43 (d, J = 7.9 Hz, 1H), |
| | | Film) | (m/z) | 8.29 (d, J = 5.4 Hz, 1H), 7.30 (dd, J = 8.7, 7.3 Hz, 2H), |
| | | 3376, | ([M + H]⁺) | 7.00-6.91 (m, 4H), 5.75 (d, J = 1.4 Hz, 2H), 5.33- |
| | | 2968, | calcd for | 5.25 (m, 1H), 4.69 (dt, J = 9.9, 7.6 Hz, 1H), 4.34 (t, J = |
| | | 2936, | C₂₆H₃₃N₂O₉, | 9.2 Hz, 1H), 3.91 (s, 3H), 3.72-3.56 (m, 3H), 2.49- |
| | | 2877, | 517.2181; | 2.41 (m, 1H), 2.07 (s, 3H), 1.80 (dtd, J = 13.5, 10.2, 3.0 |
| | | 1752, | found, | Hz, 1H), 1.65-1.57 (m, 2H), 1.32 (d, J = 6.6 Hz, 3H), |
| | | 1675, | 517.2186 | 0.96 (t, J = 7.3 Hz, 3H) |
| | | 1492 | | |
| 91 | — | (Thin | HRMS-ESI | ¹H NMR (500 MHz, CDCl₃) δ 8.42 (d, J = 8.0 Hz, 1H), |
| | | Film) | (m/z) | 8.29 (d, J = 5.4 Hz, 1H), 7.01-6.94 (m, 3H), 6.90- |
| | | 3376, | ([M + H]⁺) | 6.86 (m, 2H), 5.75 (d, J = 1.4 Hz, 2H), 5.27 (p, J = 6.6 |
| | | 2937, | calcd for | Hz, 1H), 4.68 (dt, J = 9.8, 7.5 Hz, 1H), 4.23 (t, J = 9.2 |
| | | 2879, | C₂₆H₃₂FN₂O₉, | Hz, 1H), 3.92 (s, 3H), 3.70-3.55 (m, 3H), 2.51-2.36 |
| | | 1752, | 535.2086; | (m, 1H), 2.07 (s, 3H), 1.86-1.73 (m, 1H), 1.67-1.56 |
| | | 1675, | found, | (m, 2H), 1.32 (d, J = 6.6 Hz, 3H), 0.97 (t, J = 7.3 Hz, |
| | | 1501 | 535.2094 | 3H) |
| 92 | — | (Thin | HRMS-ESI | ¹H NMR (500 MHz, CDCl₃) δ 8.62 (s, 1H), 8.34 (d, J = |
| | | Film) | (m/z) | 5.4 Hz, 1H), 7.00 (d, J = 5.5 Hz, 1H), 5.17-5.05 (m, |
| | | 3377, | ([M + H]⁺) | 1H), 4.97 (s, 1H), 4.87 (s, 1H), 4.62 (ddd, J = 9.9, 8.2, |
| | | 2970, | calcd for | 7.3 Hz, 1H), 4.06 (d, J = 11.5 Hz, 1H), 3.90 (s, 3H), |
| | | 2937, | C₂₃H₃₃N₂O₈, | 3.90 (d, J = 11.5 Hz, 1H), 3.62-3.47 (m, 2H), 3.40 |
| | | 2877, | 465.2231; | (ddd, J = 11.5, 9.3, 2.6 Hz, 1H), 3.21 (t, J = 9.3 Hz, 1H), |
| | | 1770, | found, | 2.39 (s, 3H), 2.38-2.32 (m, 1H), 1.74 (s, 3H), 1.81- |
| | | 1748, | 465.2225 | 1.66 (m, 2H), 1.65-1.56 (m, 1H), 1.42 (d, J = 6.5 Hz, |
| | | 1676, | | 3H), 0.98 (t, J = 7.3 Hz, 3H) |
| | | 1507 | | |
| 93 | — | (Thin | HRMS-ESI | ¹H NMR (500 MHz, CDCl₃) δ 8.63 (s, 1H), 8.33 (d, J = |
| | | Film) | (m/z) | 5.4 Hz, 1H), 6.99 (d, J = 5.5 Hz, 1H), 5.13-5.03 (m, |
| | | 3377, | ([M + H]⁺) | 1H), 4.61 (ddd, J = 9.9, 8.3, 7.3 Hz, 1H), 3.90 (s, 3H), |
| | | 2959, | calcd for | 3.61-3.47 (m, 2H), 3.41 (dd, J = 8.3, 6.6 Hz, 1H), 3.40- |
| | | 2876, | C₂₃H₃₅N₂O₈, | 3.29 (m, 1H), 3.24 (dd, J = 8.3, 6.3 Hz, 1H), 3.12 (t, |
| | | 1770, | 467.2388; | J = 9.3 Hz, 1H), 1.63-1.49 (m, 1H), 2.39 (s, 3H), 2.40- |
| | | 1747, | found, | 2.33 (m, 1H), 1.80 (dh, J = 13.2, 6.6 Hz, 1H), 1.75- |
| | | 1676, | 467.2389 | 1.66 (m, 2H), 1.40 (d, J = 6.5 Hz, 3H), 0.98 (t, J = 7.4 |
| | | 1507 | | Hz, 3H), 0.91 (d, J = 6.7 Hz, 3H), 0.90 (d, J = 6.7 Hz, |
| | | | | 3H) |
| 94 | — | (Thin | HRMS-ESI | ¹H NMR (500 MHz, CDCl₃) δ 8.65 (d, J = 8.2 Hz, 1H), |
| | | Film) | (m/z) | 8.35 (d, J = 5.4 Hz, 1H), 7.29 (dd, J = 8.7, 7.3 Hz, 2H), |
| | | 3376, | ([M + H]⁺) | 7.01 (d, J = 5.5 Hz, 1H), 7.01-6.93 (m, 1H), 6.95- |
| | | 2969, | calcd for | 6.92 (m, 2H), 5.28 (dt, J = 13.1, 6.5 Hz, 1H), 4.67 (ddd, |
| | | 2936, | C₂₅H₃₁N₂O₈, | J = 9.9, 8.2, 7.3 Hz, 1H), 4.32 (t, J = 9.2 Hz, 1H), 3.91 |
| | | 2878, | 487.2075; | (s, 3H), 3.71-3.54 (m, 3H), 2.50-2.40 (m, 1H), 2.40 |
| | | 1768, | found, | (s, 3H), 1.78 (dtd, J = 13.6, 9.9, 3.3 Hz, 1H), 1.69-1.56 |
| | | 1675, | 487.2081 | (m, 2H), 1.31 (d, J = 6.6 Hz, 3H), 0.95 (t, J = 7.3 Hz, |
| | | 1507, | | 3H) |
| | | 1492 | | |
| 95 | — | (Thin | HRMS-ESI | ¹H NMR (400 MHz, CDCl₃) δ 8.60 (d, J = 8.4 Hz, 1H), |
| | | Film) | (m/z) | 8.33 (d, J = 5.5 Hz, 1H), 7.00 (d, J = 5.5 Hz, 1H), 5.23 |
| | | 3372, | ([M + H]⁺) | (dq, J = 7.6, 6.4 Hz, 1H), 5.02 (t, J = 9.6 Hz, 1H), 4.66 |
| | | 2972, | calcd for | (ddt, J = 10.2, 8.3, 5.2 Hz, 1H), 3.90 (s, 3H), 3.70-3.54 |
| | | 2938, | C₂₃H₃₃N₂O₉, | (m, 2H), 3.49 (ddd, J = 11.7, 9.7, 2.6 Hz, 1H), 2.57 (p, |
| | | 1879, | 481.2181; | J = 7.0 Hz, 1H), 2.46-2.35 (m, 1H), 2.39 (s, 3H), 1.82- |
| | | 1770, | found, | 1.68 (m, 1H), 1.59 (ddt, J = 14.3, 11.4, 7.2 Hz, 1H), |
| | | 1739, | 481.2181 | 1.36-1.27 (m, 1H), 1.24 (d, J = 6.5 Hz, 3H), 1.18 (d, |
| | | 1676, | | J = 7.0 Hz, 3H), 1.18 (d, J = 7.0 Hz, 3H), 0.94 (t, J = 7.4 |
| | | 1507 | | Hz, 3H) |
| 96 | 79- | — | HRMS-ESI | ¹H NMR (400 MHz, CDCl₃) δ 8.55 (d, J = 7.5 Hz, 1H), |
| | 81 | | (m/z) | 8.28 (d, J = 5.3 Hz, 1H), 7.40-7.25 (m, 5H), 6.95 (d, |
| | | | ([M + H]⁺) | J = 5.4 Hz, 1H), 5.78-5.70 (m, 2H), 5.28 (p, J = 6.6 Hz, |
| | | | calcd for | 1H), 4.70 (td, J = 7.6, 5.6 Hz, 1H), 4.62 (d, J = 12.0 Hz, |
| | | | C₂₅H₃₁N₂O₉, | 1H), 4.58 (d, J = 12.0 Hz, 1H), 3.93-3.83 (m, 4H), |
| | | | 503.2024; | 3.77 (dd, J = 11.8, 5.5 Hz, 1H), 3.62 (dd, J = 11.8, 3.6 |
| | | | found, | Hz, 1H), 3.56-3.45 (m, 2H), 2.47 (dddd, J = 14.5, 7.6, |
| | | | 503.2037 | 5.7, 2.3 Hz, 1H), 2.06 (s, 3H), 1.89 (app dtd, J = 14.4, |
| | | | | 7.9, 2.6 Hz, 1H), 1.36 (d, J = 6.7 Hz, 3H) |
| 97 | 111- | — | HRMS-ESI | ¹H NMR (400 MHz, CDCl₃) δ 8.53 (d, J = 7.5 Hz, 1H), |
| | 112 | | (m/z) | 8.28 (d, J = 5.3 Hz, 1H), 6.95 (d, J = 5.4 Hz, 1H), 5.78- |
| | | | ([M + H]⁺) | 5.70 (m, 2H), 5.18 (p, J = 6.7 Hz, 1H), 4.69 (td, J = 7.8, |
| | | | calcd for | 5.9 Hz, 1H), 3.91 (s, 3H), 3.85 (ddd, J = 10.8, 7.0, 2.5 |
| | | | C₂₂H₃₃N₂O₉, | Hz, 1H), 3.71-3.64 (m, 2H), 3.54 (ddd, J = 10.6, 8.1, |
| | | | 469.2181; | 2.1 Hz, 1H), 3.40 (app dt, J = 7.2, 5.1 Hz, 1H), 3.31 (dd, |
| | | | found, | J = 8.9, 6.2 Hz, 1H), 3.20 (dd, J = 8.9, 6.9 Hz, 1H), 2.47 |
| | | | 469.2187 | (dddd, J = 14.9, 7.0, 5.9, 2.2 Hz, 1H), 2.07 (s, 3H), 1.94- |
| | | | | 1.75 (m, 2H), 1.41 (d, J = 6.6 Hz, 3H), 0.91 (d, J = 6.7 |
| | | | | Hz, 3H), 0.89 (d, J = 6.7 Hz, 3H) |
| 98 | 115- | — | HRMS-ESI | ¹H NMR (500 MHz, CDCl₃) δ 8.50 (d, J = 7.5 Hz, 1H), |
| | 117 | | (m/z) | 8.28 (d, J = 5.3 Hz, 1H), 6.95 (d, J = 5.4 Hz, 1H), 5.74 |
| | | | ([M + H]⁺) | (app d, J = 1.8 Hz, 2H), 5.27 (qd, J = 6.7, 5.1 Hz, 1H), |
| | | | calcd for | 4.80 (td, J = 5.2, 3.3 Hz, 1H), 4.76-4.68 (m, 1H), 4.02- |
| | | | C₂₂H₃₁N₂O₁₀, | 3.94 (m, 1H), 3.91 (s, 3H), 3.77 (dd, J = 12.0, 5.1 Hz, |
| | | | 483.1973; | 1H), 3.69 (dd, J = 12.1, 3.3 Hz, 1H), 3.59-3.51 (m, |
| | | | found, | 1H), 2.61 (hept, J = 7.0 Hz, 1H), 2.51-2.41 (m, 1H), |
| | | | 483.1974 | 2.07 (s, 3H), 1.90 (dtd, J = 14.6, 8.6, 2.6 Hz, 1H), 1.39 |
| | | | | (d, J = 6.7 Hz, 3H), 1.21 (d, J = 7.0 Hz, 3H), 1.17 (d, |
| | | | | J = 7.0 Hz, 3H) |
| 99 | 110- | — | HRMS-ESI | ¹H NMR (400 MHz, CDCl₃) δ 8.55 (d, J = 7.5 Hz, 1H), |
| | 113 | | (m/z) | 8.29 (d, J = 5.3 Hz, 1H), 7.34-7.24 (m, 2H), 7.03- |
| | | | ([M + H]⁺) | 6.89 (m, 4H), 5.79-5.71 (m, 2H), 5.40 (p, J = 6.6 Hz, |
| | | | calcd for | 1H), 4.74 (td, J = 7.9, 5.9 Hz, 1H), 4.42-4.33 (m, 1H), |
| | | | C₂₄H₂₉N₂O₉, | 3.98-3.90 (m, 4H), 3.88 (dd, J = 12.0, 5.7 Hz, 1H), |
| | | | 489.1868; | 3.81 (dd, J = 12.0, 3.9 Hz, 1H), 3.60 (ddd, J = 10.7, 8.4, |
| | | | found, | 2.1 Hz, 1H), 2.49 (app dtd, J = 14.7, 6.5, 2.1 Hz, 1H), |
| | | | 489.1882 | 2.07 (s, 3H), 1.93 (app dtd, J = 14.5, 8.3, 2.6 Hz, 1H), |
| | | | | 1.44 (d, J = 6.6 Hz, 3H) |
| 100 | 69- | — | HRMS-ESI | ¹H NMR (400 MHz, CDCl₃) δ 8.76 (d, J = 7.6 Hz, 1H), |
| | 74 | | (m/z) | 8.34 (d, J = 5.4 Hz, 1H), 7.33-7.24 (m, 2H), 7.04- |
| | | | ([M + H]⁺) | 6.89 (m, 4H), 5.40 (p, J = 6.6 Hz, 1H), 4.71 (td, J = 7.9, |
| | | | calcd for | 5.9 Hz, 1H), 4.41-4.32 (m, 1H), 3.97-3.83 (m, 5H), |
| | | | C₂₃H₂₇N₂O₈, | 3.80 (dd, J = 12.0, 3.8 Hz, 1H), 3.57 (ddd, J = 10.6, 8.3, |
| | | | 459.1762; | 2.0 Hz, 1H), 2.52-2.37 (m, 4H), 1.97-1.83 (m, 1H), |
| | | | found, | 1.43 (d, J = 6.6 Hz, 3H) |
| | | | 459.1767 | |
| 101 | 43- | — | HRMS-ESI | ¹H NMR (400 MHz, CDCl₃) δ 8.76 (d, J = 7.4 Hz, 1H), |
| | 48 | | (m/z) | 8.33 (d, J = 5.4 Hz, 1H), 7.00 (d, J = 5.5 Hz, 1H), 5.20 |
| | | | ([M + H]⁺) | (p, J = 6.6 Hz, 1H), 4.67 (td, J = 7.6, 5.6 Hz, 1H), 3.93- |
| | | | calcd for | 3.83 (m, 4H), 3.73 (dd, J = 11.8, 5.2 Hz, 1H), 3.67- |
| | | | C₂₁H₂₈F₃N₂O₈, | 3.54 (m, 2H), 3.54-3.44 (m, 2H), 3.37 (ddd, J = 6.3, |
| | | | 493.1792; | 5.2, 3.5 Hz, 1H), 2.52-2.36 (m, 4H), 2.29-2.11 (m, |
| | | | found, | 2H), 1.94-1.77 (m, 3H), 1.39 (d, J = 6.7 Hz, 3H) |
| | | | 493.1798 | |

*Cmpd. No.—Compound Number

TABLE 3
Biological Testing Rating Scale
Rating Table for Fungal Pathogens

| % Control | Rating |
|---|---|
| >70 | A |
| ≦70 | B |
| Not Tested | C |

TABLE 4
Biological Activity - PUCCRT and SEPTTR Disease
Control in High and Low Volume Applications

| | Low Volume | | | | High Volume | | | |
|---|---|---|---|---|---|---|---|---|
| | (121.5 g/H*) | | | | (100 ppm*) | | | |
| *Cmpd. | PUCCRT* | | SEPTTR* | | PUCCRT* | | SEPTTR* | |
| No. | 1DP* | 3DC* | 1DP* | 3DC* | 1DP* | 3DC* | 1DP* | 3DC* |
| 51 | C | C | C | C | A | A | A | B |
| 52 | C | C | C | C | A | A | A | A |
| 53 | C | C | C | C | A | A | A | A |
| 54 | C | C | C | C | A | A | A | A |
| 55 | C | C | C | C | A | A | A | A |
| 56 | C | C | C | C | A | A | A | A |
| 57 | C | C | C | C | A | A | A | A |
| 58 | C | C | C | C | A | A | A | A |
| 59 | C | C | C | C | A | A | B | A |
| 60 | C | C | C | C | A | A | A | A |
| 61 | C | C | C | C | A | A | B | A |
| 62 | C | C | C | C | A | A | B | A |
| 63 | C | C | C | C | A | A | A | A |
| 64 | C | C | C | C | A | A | A | A |
| 65 | C | C | C | C | A | A | B | B |
| 66 | C | C | C | C | A | A | B | B |
| 67 | C | C | C | C | A | A | A | A |
| 68 | C | C | C | C | A | A | B | B |
| 69 | C | C | C | C | A | A | A | A |
| 70 | C | C | C | C | B | B | B | B |
| 71 | A | A | A | A | C | C | C | C |
| 72 | A | A | A | A | C | C | C | C |
| 73 | A | A | A | A | C | C | C | C |
| 74 | A | A | A | A | C | C | C | C |
| 75 | A | A | A | B | C | C | C | C |
| 76 | A | A | A | A | C | C | C | C |
| 77 | A | A | A | A | C | C | C | C |
| 78 | A | A | B | B | C | C | C | C |
| 79 | A | A | A | A | C | C | C | C |
| 80 | B | B | B | B | C | C | C | C |
| 81 | B | B | B | B | C | C | C | C |
| 82 | A | A | A | A | C | C | C | C |
| 83 | A | A | A | A | C | C | C | C |
| 84 | C | C | C | C | C | C | C | C |
| 85 | A | A | A | A | C | C | C | C |
| 86 | A | A | A | B | C | C | C | C |
| 87 | A | A | A | B | C | C | C | C |
| 88 | A | A | B | B | C | C | C | C |
| 89 | A | A | A | A | C | C | C | C |
| 90 | A | A | A | A | C | C | C | C |
| 91 | A | A | A | A | C | C | C | C |
| 92 | A | A | B | B | C | C | C | C |
| 93 | A | A | A | A | C | C | C | C |
| 94 | A | A | A | A | C | C | C | C |
| 95 | A | A | B | A | C | C | C | C |
| 96 | A | A | B | A | C | C | C | C |
| 97 | A | A | A | A | C | C | C | C |
| 98 | B | B | B | B | C | C | C | C |
| 99 | A | A | B | B | C | C | C | C |
| 100 | A | A | B | A | C | C | C | C |
| 101 | A | A | B | B | C | C | C | C |

*Cmpd. No.—Compound Number
*PUCCRT—Wheat Brown Rust (Puccinia triticina)
*SEPTTR—Wheat Leaf Blotch (Zymoseptoria tritici)
*1DP—1 Day Protectant
*3DC—3 Day Curative
*g/H—Grams Per Hectare
*ppm—Parts Per Million

TABLE 5
Biological Activity - Disease Control at 100 ppm

| *Cmpd. | ALTESO* | CERCBE* | COLLLA* | ERYSCI* | ERYSGH* |
|---|---|---|---|---|---|
| No. | 1DP* | | | | |
| 51-101 | C | C | C | C | C |

*Cmpd. No.—Compound Number
*ALTESO - Tomato Early Blight (Alternaria solani)
*CERCBE - Leaf Spot of Sugar Beets (Cercospora beticola)
*COLLLA - Cucumber Anthracnose (Glomerella lagenarium; Anamorph: Colletotricum lagenarium)
*ERYSCI - Powdery Mildew of Cucumber (Erysiphe cichoracearum)
*ERYSGH - Barley Powdery Mildew (Blumeria graminis f.sp. hordei; Synonym: Erysiphe graminis f.sp. hordei)
*1DP—1 Day Protectant

TABLE 6
Biological Activity - Disease Control at 100 ppm

| | | | | |
|---|---|---|---|---|
| *Cmpd. | LEPTNO* | RHYNSE* | UNCINE* | VENTIN* |

| | |
|---|---|
| No. | 1DP |

| | | | | |
|---|---|---|---|---|
| 51-101 | C | C | C | C |

*Cmpd. No.—Compound Number
*LEPTNO - Wheat Glume Blotch (Leptosphaeria nodorum)
*RHYNSE - Barley Scald (Rhyncosporium secalis)
*UNCINE - Grape Powdery Mildew (Uncinula necator)
*VENTIN - Apple Scab (Venturia inaequalis)
*1DP—1 Day Protectant

TABLE 7
Biological Activity - Disease Control at 25 ppm

| | |
|---|---|
| *Cmpd. | PHAKPA* |

| No. | 1DP* | 3DC* |
|---|---|---|
| 51-101 | C | C |

*Cmpd. No.—Compound Number
*PHAKPA - Asian Soybean Rust (Phakopsora pachyrhizi)
*1DP—1 Day Protectant
*3DC—3 Day Curative

What is claimed is:

1. A compound of Formula I 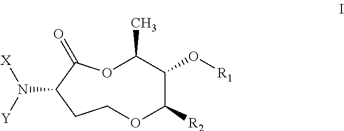
wherein
X is hydrogen or C(O)R₃;
Y is hydrogen, C(O)R₃, or Q;
Q is 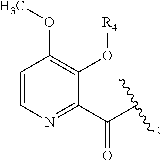
R₁ is hydrogen, alkyl, aryl, acyl, or silyl each optionally substituted with 0, 1 or multiple R₆;
R₂ is —(CH₂)ₙR₈ where n is an integer between 0 and 4, each optionally substituted with 0, 1 or multiple R₆;
R₃ is alkoxy or benzyloxy, each optionally substituted with 0, 1, or multiple R₆;
R₄ is hydrogen, —C(O)R₅, or —CH₂OC(O)R₅;
R₅ is alkyl, alkoxy, or aryl, each optionally substituted with 0, 1, or multiple R₆;
R₆ is hydrogen, alkyl, aryl, acyl, halo, alkenyl, alkoxy, heterocyclyl, or thioalkyl, each optionally substituted with 0, 1, or multiple R₇;
R₇ is hydrogen, alkyl, aryl, alkoxy, or halo; and
R₈ is hydrogen, alkyl, alkenyl, aryl, heterocyclyl, or thioalkyl each substituted with 0, 1, or multiple R₆.

2. A compound according to claim 1, wherein X and Y are hydrogen.

3. A compound according to claim 2, wherein R₁ is alkyl, aryl, or acyl each optionally substituted with 0, 1 or multiple R₆.

4. A compound according to claim 2, wherein R₂ is chosen from —(CH₂)ₙR₈ where n is an integer between 0 and 4 and R₈ is hydrogen or alkyl, optionally substituted with 0, 1 or multiple R₆.

5. A compound according to claim 1, wherein X is C(O)R₃ and Y is hydrogen.

6. A compound according to claim 5, wherein R₁ is chosen from hydrogen, alkyl, aryl, acyl, or silyl each optionally substituted with 0, 1 or multiple R₆.

7. A compound according to claim 5, wherein R₂ is chosen from —(CH₂)ₙR₈ where n is an integer between 0 and 4 and R₈ is hydrogen or alkyl, optionally substituted with 0, 1 or multiple R₆.

8. A compound according to claim 1, wherein X is hydrogen and Y is Q.

9. A compound according to claim 8, wherein R₁ is chosen from alkyl, aryl, or acyl each optionally substituted with 0, 1 or multiple R₆.

10. A compound according to claim 8, wherein R₂ is chosen from —(CH₂)ₙR₈ where n is an integer between 0 and 4 and R₈ is hydrogen or alkyl, optionally substituted with 0, 1 or multiple R₆.

11. A compound according to claims 9 and 10, wherein R₄ is hydrogen.

12. A compound according to claims 9 and 10, wherein R₄ is —C(O)R₅ or —CH₂OC(O)R₈.

13. A compound according to claim 12, wherein R₅ is chosen from alkyl or alkoxy, each optionally substituted with 0, 1, or multiple R₆.

14. A compound according to claim 13, wherein R₅ is chosen from —CH₃ or —CH(CH₃)₂.

15. A composition for the control of a fungal pathogen including at least one of the compounds of claims 1-14 and a phytologically acceptable carrier material.

16. A composition for the control of a fungal pathogen including mixtures of at least one of the compounds of claims 1-14 with other pesticides including fungicides, insecticides, nematocides, miticides, arthropodicides, bactericides, and combinations thereof.

17. The compositions according to claims 15-16 wherein the fungal pathogen is one of Leaf Blotch of Wheat (Mycosphaerella graminicola; anamorph: Zymoseptoria tritici), Wheat Brown Rust (Puccinia triticina), Stripe Rust (Puccinia striiformis), Scab of Apple (Venturia inaequalis), Powdery Mildew of Grapevine (Uncinula necator), Barley Scald (Rhynchosporium secalis), Blast of Rice (Magnaporthe grisea), Rust of Soybean (Phakopsora pachyrhizi), Glume Blotch of Wheat (Leptosphaeria nodorum), Powdery Mildew of Wheat (Blumeria graminis f. sp. tritici), Powdery Mildew of Barley (Blumeria graminis f. sp. hordei), Powdery Mildew of Cucurbits (Erysiphe cichoracearum), Anthracnose of Cucurbits (Glomerella lagenarium), Leaf Spot of Beet (Cercospora beticola), Early Blight of Tomato (Alternaria solani), and Net Blotch of Barley (Pyrenophora teres).

18. The composition according to claim 17 wherein the fungal pathogen is one of Leaf Blotch of Wheat (Zymoseptoria tritici), Wheat Brown Rust (Puccinia triticina), and Rust of Soybean (Phakopsora pachyrhizi).

19. A method for the control and prevention of fungal attack on a plant, the method including the step of: Applying a fungicidally effective amount of at least one of the compounds of claims 1-14 or at least one of the compositions according to claims 15-16 to at least one of the plant, an area adjacent to the plant, soil adapted to support growth of the plant, a root of the plant, and foliage of the plant.
